(19) United States
(12) Patent Application Publication
(10) Pub. No.: US 2025/0325531 A1
(43) Pub. Date: Oct. 23, 2025

(54) TREATMENT OF SYSTOLIC DYSFUNCTION

(71) Applicant: MyoKardia, Inc., Brisbane, CA (US)

(72) Inventors: Jean-Francois Tamby, San Mateo, CA (US); Chun Yang, San Mateo, CA (US); Timothy Carlson, Belmont, CA (US)

(73) Assignee: MyoKardia, Inc., Brisbane, CA (US)

(21) Appl. No.: 18/639,017

(22) Filed: Apr. 18, 2024

(51) Int. Cl. A61K 31/454

(52) U.S. Cl. CPC A61K 31/454

(57) ABSTRACT

Provided herein are methods, use, and compositions for treating systolic dysfunction such as heart failure with reduced ejection fraction.

### CROSS-REFERENCE TO RELATED APPLICATIONS

This application claims priority from U.S. Provisional Patent Application 62/849,936, filed May 19, 2019, and U.S. Provisional Patent Application 62/852,739, filed May 24, 2019. The disclosures of these priority applications are incorporated by reference herein in their entirety.

### BACKGROUND OF THE INVENTION

Heart failure (HF) is a global pandemic affecting about 26 million people worldwide. It is the most rapidly growing cardiovascular condition globally, with substantial morbidity, mortality, and cost burden to healthcare systems (Ponikowski et al., ESC Heart Fail. (2014) 1(1):4-25; Savarese and Lund, Card Fail Rev. (2017) 3(1):7-11). HF is the most common cause of hospitalization in patients older than 65 years (Ponikowski, supra; Savarese and Lund, supra; and Shah et al., J Am Coll Cardiol. (2017) 70(20):2476-86). The five-year mortality rate after HF hospitalization is about 42%, comparable to many cancers (Benjamin et al., Circulation (2019) 139:e56-e528).

Heart failure is a clinical syndrome in which a patient's heart is unable to provide an adequate supply of blood flow to the body to meet the body's metabolic needs. For some patients with heart failure, the heart has difficulty pumping enough blood to support other organs in the body. Other patients may have a hardening and stiffening of the heart muscle itself, which blocks or reduces blood flow to the heart. Those two conditions result in inadequate blood circulation to the body and congestion of the lungs. Heart failure can affect the right or left side of the heart, or both sides at the same time. It can be either an acute (short-term) or chronic (ongoing) condition. Heart failure can be referred to as congestive heart failure when fluid builds up in various parts of the body. Symptoms of heart failure include, but are not limited to, excessive fatigue, sudden weight gain, a loss of appetite, persistent coughing, irregular pulse, chest discomfort, angina, heart palpitations, edema (e.g., swelling of the lungs, arms, legs, ankles, face, hands, or abdomen), shortness of breath (dyspnea), protruding neck veins, and decreased exercise tolerance or capacity.

The volume of blood pumped by the heart is generally determined by: (a) the contraction of the heart muscle (i.e., how well the heart squeezes or its systolic function) and (b) the filling of the heart chambers (i.e., how well the heart relaxes and fills with blood or its diastolic function). Ejection fraction is used to assess the pump function of the heart; it represents the percentage of blood pumped from the left ventricle (the main pumping chamber) per beat. A normal or preserved ejection fraction is greater than or equal to 50 percent. If the systolic function of the heart is impaired such that the heart demonstrates substantial reduction in ejection fraction (i.e., an ejection fraction of <50%), this condition is known as heart failure with reduced ejection fraction (HFrEF). HFrEF with an ejection fraction of ≤40% is classical HFrEF, while HFrEF with an ejection fraction of 41-49% is classified as heart failure with mid-range ejection fraction (HFmrEF), under the 2013 American College of Cardiology Foundation/American Heart Association guidelines (Yancy et al., Circulation (2013) 128:e240-327) and the 2019 ACC Expert Consensus Decision Pathway on Risk Assessment, Management, and Clinical Trajectory of Patients Hospitalized With Heart Failure (Hollenberg et al., J Am Coll Cardiol (2019) 74:1966 2011). There are many causes for a weak heart muscle (low ejection fraction), including ischemia/infarction, hypertension, heart valve defects, gene mutations, infection, and toxin/drug exposure.

Diastolic dysfunction may contribute to morbidity in HFrEF patients. If the heart pumps normally but is too stiff to fill properly, this condition is known as heart failure with preserved ejection fraction (HFpEF). Historically, HFpEF was termed diastolic heart failure; however, recent investigations suggest a more complex and heterogeneous pathophysiology. HFpEF patients exhibit subtle or mild abnormalities in systolic performance, which become more dramatic during exercise. Ventricular diastolic and systolic reserve abnormalities, chronotropic incompetence, stiffening of ventricular tissue, atrial dysfunction, pulmonary hypertension, impaired vasodilation, and endothelial dysfunction are all implicated. Frequently, these abnormalities are noted only when the circulatory system is stressed.

In the United States alone, there are about 2.6 million HFrEF patients, corresponding to about 40% of the U.S. HF population (Bloom et al., Nat Rev Dis Primers. (2017) 3:17058). HFrEF may develop from an ischemic origin (primarily attributed to coronary artery disease) or a non-ischemic origin (attributed to a disease of the myocardium from non-coronary causes). Coronary artery disease (coronary heart disease) is a disease in which there is a narrowing of the passageway of the coronary arteries; when severe, the narrowing causes inadequate blood supply to the heart muscle and may lead to the death of heart muscle cells (infarction). Non-ischemic HFrEF is sometimes referred to as dilated cardiomyopathy (DCM). Despite the nomenclature, dilated (enlarged) heart chambers can be found in both non-ischemic and ischemic HFrEF patients. Hereafter, DCM refers to non-ischemic HFrEF. DCM can be assigned a clinical diagnosis of genetic DCM or “idiopathic” DCM if no identifiable cause can be found. Mutations in over 30 genes, including sarcomere genes, perturb a diverse set of myocardial proteins to cause a DCM phenotype. Some of the genetic links to DCM are discussed in Hershberger, et al., Nature Reviews (2013) 10(9):531-47 and Rosenbaum et al., Nat Rev Cardiol. (2020) 17(5):286-97.

Contemporary medical therapy for HFrEF centers on counteracting the effects of neurohormonal activation with modulators of the renin-angiotensin-aldosterone system, β-adrenergic blockers, diuretics, and modulators of the vasoactive peptide BNP (brain natriuretic peptide). Although these drugs attenuate some of the maladaptive consequences and improve clinical outcomes, none addresses the underlying causal pathways of myocardial dysfunction.

Several inotropic agents are used in clinical practice to augment cardiac contractility by increasing intracellular calcium or cyclic adenosine monophosphate, mechanisms that increase myocardial oxygen demand. Their use is limited to short-term or destination therapy in patients with refractory or end-stage heart failure for the purpose of symptom relief, as chronic studies with these drugs have demonstrated increased mortality due to arrhythmias and ischemia. However, these drugs do improve hemodynamics and symptoms, suggesting a potential clinical benefit for agents that increase contractility without arrhythmic or ischemic liabilities.

There are currently no approved therapies for treating heart failure by targeting the contractile apparatus directly. There remains an urgent need for new safe, effective treatments for systolic heart failure.

### SUMMARY OF THE INVENTION

The present disclosure provides a method of treating systolic dysfunction in a patient in need thereof, comprising orally administering to the patient Compound I at a total daily amount of 10-350 mg, wherein Compound I is (R)-4-(1-((3-(difluoromethyl)-1-methyl-1H-pyrazol-4-yl)sulfonyl)-1-fluoroethyl)-N-(isoxazol-3-yl) piperidine-1-carboxamide, having the structural formula (I)

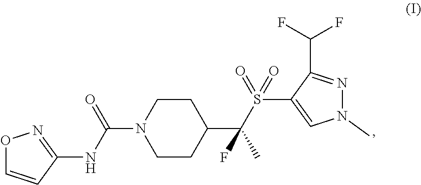 [SMILES: Cn1cc(S(=O)(=O)[C@@](C)(F)C2CCN(C(=O)Nc3ccon3)CC2)c(C(F)F)n1]

or a pharmaceutically acceptable salt thereof.

In some embodiments, the patient is suffering from a syndrome or disorder selected from the group consisting of heart failure (including, but not limited to, heart failure with reduced ejection fraction (HFrEF), heart failure with preserved ejection fraction (HFpEF), congestive heart failure, and diastolic heart failure (with diminished systolic reserve)); a cardiomyopathy (including, but not limited to, ischemic cardiomyopathy, dilated cardiomyopathy, post-infarction cardiomyopathy, viral cardiomyopathy, toxic cardiomyopathy (including, but not limited to, post-anthracycline anticancer therapy), metabolic cardiomyopathy (including, but not limited to, in conjunction with enzyme replacement therapy), infiltrative cardiomyopathy (including, but not limited to, amyloidosis), and diabetic cardiomyopathy); cardiogenic shock; conditions that benefit from inotropic support after cardiac surgery (e.g., ventricular dysfunction due to on-bypass cardiovascular surgery); myocarditis (including, but not limited to, viral); atherosclerosis; secondary aldosteronism; myocardial infarction; valve disease (including, but not limited to, mitral regurgitation and aortic stenosis); systemic hypertension; pulmonary hypertension (i.e., pulmonary arterial hypertension); detrimental vascular remodeling; pulmonary edema; and respiratory failure. In certain embodiments, the syndrome or disorder may be chronic and/or stable.

In some embodiments, the patient has heart failure and a diagnosis of any one of NYHA Class II-IV. In certain embodiments, the patient has symptomatic heart failure. In some embodiments, the patient has acute heart failure.

The present disclosure also provides a method of treating heart failure with reduced ejection fraction (HFrEF) in a patient in need thereof, comprising orally administering to the patient Compound I at a total daily amount of 10-350 mg. Patients with HFrEF exhibit an ejection fraction of <50%. HFrEF with an ejection fraction of <40% is classical HFrEF, while HFrEF with an ejection fraction of 41-49% is classified as heart failure with mid-range ejection fraction (HFmrEF). In some embodiments, the patient with HFrEF also exhibits mitral regurgitation. In some embodiments, the HFrEF is ischemic HFrEF. In some embodiments, the HFrEF is dilated cardiomyopathy (DCM); optionally, the patient has a genetic predisposition to DCM or genetic DCM (which may be caused by a pathogenic or likely pathogenic variant of a gene related to cardiac function including, but not limited to, MYH7 or Titin mutation).

In some embodiments, the patient has a left ventricular ejection fraction (LVEF) less than 50%. In certain embodiments, the patient has an LVEF less than 40%, less than 35%, less than 30%, between 15-35%, between 15-40% (e.g., between 15-39%), between 15-49%, between 20-45%, between 40-49%, or between 41-49%.

In some embodiments, the patient has an elevated NT-proBNP level. In certain embodiments, the patient has an NT-proBNP level of greater than 400 μg/mL.

In some embodiments, the patient does not have any one or combination of the following:
a) current angina pectoris;
b) recent (<90 days) acute coronary syndrome diagnosis;
c) coronary revascularization (percutaneous coronary intervention [PCI] or coronary artery bypass graft [CABG]) within the prior 3 months; and
d) uncorrected severe valvular disease.

In some embodiments, the treatment results in any one or combination of the following:
a) reduced risk of cardiovascular mortality;
b) reduced risk of cardiovascular-related hospitalization (including, but not limited to, worsening heart failure);
c) improved exercise capacity;
d) improvement in a patient's NYHA classification;
e) delay in clinical worsening; and
f) reduction in severity of cardiovascular-related symptoms.

In some embodiments, the exercise capacity improvement is a >3 mL/kg/min improvement in peak VO₂ (pVO₂). In some embodiments, the treatment results comprise an improvement in NYHA Class (e.g., from Class IV to Class III, from Class III to Class II, Class II to Class I, or from Class I to no heart failure) and an improvement in exercise capacity as measured by pVO₂ (e.g., wherein the pVO₂ improvement is a >1.5 mL/kg/min improvement) or activity as measured by accelerometry. Cardiovascular-related symptoms may include, e.g., excessive fatigue, sudden weight gain, a loss of appetite, persistent coughing, irregular pulse, chest discomfort, angina, heart palpitations, edema (e.g., swelling of the lungs, arms, legs, ankles, face, hands, or abdomen), shortness of breath (dyspnea), protruding neck veins, decreased exercise tolerance or capacity, and any combination thereof.

In some embodiments, the treatment method results in reduction of the risk of cardiovascular death and hospitalization for heart failure in patients with chronic heart failure (NYHA Class II-IV) and reduced ejection fraction.

In some embodiments, the present treatment method reduces the risk of hospitalization for worsening heart failure in patients with stable, symptomatic chronic HFrEF.

In some embodiments, the treatment improves survival, prolongs time to hospitalization for heart failure and improves patient-reported functional status in patients with systolic heart failure.

In some embodiments, the present treatment method increases left ventricular ejection fraction and improves heart failure symptoms, as evidenced by improved exercise capacity and decreased heart failure-related hospitalizations and emergency care.

Any combination of the above treatment results is also contemplated.

In some embodiments, the patient is administered Compound I at 10-175 mg BID (e.g., 10-75 mg or 25-75 mg BID such as 10, 25, 50, or 75 mg BID), 25-325 mg QD (e.g., 75-125 mg QD), or 25-350 mg QD. In some embodiments, the Compound I is ingested by the patient with food, or within about two hours, within one hour, or within 30 minutes of food. In some embodiments, the Compound I is provided in a solid form with a mean particle size of greater than 15 μm or between 15-25 μm in diameter. In some embodiments, the QD dosing is greater than 200 mg.

In some embodiments, the patient is administered Compound I in a solid form with a mean particle size of less than 10 μm in diameter. In certain embodiments, the mean particle size is between 1-10 μm in diameter or 1-5 μm in diameter.

In some embodiments, the patient
a) is administered a loading dose of 50-250 mg; and
b) continues with a BID or QD maintenance dosing regimen approximately 10-12 hours thereafter. In certain embodiments, the BID maintenance dosing regimen is 10-75 mg BID (e.g., 10, 25, 50, or 75 mg BID) and the QD maintenance dosing regimen is 75-125 mg QD.

In some embodiments, the Compound I dose administered to the patient results in Compound I plasma concentrations of 1000 to 8000 ng/mL, e.g., <2000 ng/mL, 1000-4000 ng/mL, >2000 ng/mL, 2000-3500 ng/mL, 2000-4000 ng/ml, or >3500 ng/mL.

In some embodiments, the patient has right ventricular heart failure. In certain embodiments, the patient has pulmonary hypertension (i.e., pulmonary arterial hypertension). In some embodiments, the patient has left ventricular heart failure.

In some embodiments, administration of Compound I to the patient results in improvement of left ventricular function in the patient. A parameter of the improved left ventricular function may be selected from, e.g., improved cardiac contractility as indicated by increased ejection fraction, increased fractional shortening, increased stroke volume, increased cardiac output, improvement in global longitudinal or circumferential strain, and/or decreased left ventricular end-systolic and/or end-diastolic dimensions.

In some embodiments, administration of Compound I to the patient results in improved functional or exercise capacity of the patient as measured by peak VO₂ (e.g., improvement of >1.5 or 3 mL/kg/min), reduction in dyspnea, improvement in NYHA Class, and/or improvement in 6-minute walk test or activity (as determined by accelerometry). In certain embodiments, administration of Compound I to the patient results in improvement in NYHA Class and improvement in exercise capacity (e.g., >1.5 mL/kg/min).

In some embodiments, the patient is further administered an additional medication for improving cardiovascular conditions in the patient. The additional medication may be, e.g., a beta blocker, a diuretic (e.g., a loop diuretic), an angiotensin-converting enzyme (ACE) inhibitor, an aldosterone antagonist, a calcium channel blocker, an angiotensin II receptor blocker, a mineralocorticoid receptor antagonist (e.g. spironolactone), an ARNI, a RAAS inhibitor, an sGC activator or modulator (e.g., vericiguat), or an antiarrhythmic medication. In particular embodiments, the additional medication is an ARNI such as sacubitril/valsartan or an SGLT2 inhibitor (e.g. dapagliflozin).

In some embodiments, the patient is further administered an analgesic if the patient experiences headache.

In some embodiments, the patient is monitored for NT-proBNP levels, sinus tachycardia, ventricular tachycardia, or palpitation.

The present disclosure also provides a kit for treating systolic dysfunction (e.g., HFrEF) in a patient in need thereof, comprising Compound I in the form of tablets or capsules for oral administration, wherein each tablet or capsule may contain 5, 25, 50, 75, or 100 mg Compound I, and wherein the kit optionally includes a loading dose tablet or capsule. In some embodiments, the kit is for treating a patient according to a method described herein.

The present disclosure also provides Compound I for use in treating systolic dysfunction (e.g., HFrEF) in a patient in need thereof, wherein Compound I is administered orally at a total daily amount of 25-350 mg. In some embodiments, the treatment is according to a method described herein.

The present disclosure also provides the use of Compound I for the manufacture of a medicament for treating systolic dysfunction (e.g., HFrEF) in a patient in need thereof, wherein the medicament is for oral administration of Compound I at a total daily amount of 25-350 mg. In some embodiments, the medicament is for treating a patient according to a method described herein.

The present disclosure also provides a composition comprising Compound I for treating systolic dysfunction (e.g., HFrEF) in a patient in need thereof, wherein the composition is for oral administration of Compound I at a total daily amount of 25-350 mg. In some embodiments, the composition is for treating a patient according to a method described herein.

The present disclosure also provides a medicament for treating systolic dysfunction (e.g., HFrEF) in a patient in need thereof, comprising Compound I in the form of tablets or capsules for oral administration, wherein each tablet or capsule comprises 5, 25, 50, 75, or 100 mg of Compound I. In some embodiments, the medicament is for treating a patient according to a method described herein.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 is a graph showing the mean Compound I plasma concentration in healthy volunteers by nominal time and treatment group.

FIG. 2 is a graph showing the dose proportionality assessment of Cₘₐₓ versus dose.

FIG. 3 is a graph showing the dose proportionality assessment of AUC_{inf} versus dose.

FIG. 4 is a graph showing the mean Compound I plasma concentrations by nominal time following oral administration of 200 mg Compound I with or without food. N=10 per fasted status. Error bars are standard error of the mean (SEM).

FIGS. 5A and 5B are schematic diagrams showing the clinical trial design for treating HFrEF with Compound I. BID, twice daily; MAD, multiple-ascending doses; SAD, single-ascending doses; SRC, Safety Review Committee.

FIG. 6 is a graph showing the mean Compound I plasma concentrations in patients with stable HFrEF by nominal time and treatment group following oral administration of single ascending doses of Compound I.

FIGS. 7A and 7B are a pair of graphs showing the individual and mean plasma concentration-time profiles after oral administration of multiple doses of Compound I to patients in MAD Cohort A (75 mg twice daily on Days 1-6, and a single dose on Day 7; fasted; Panel A) and Cohort C (75 mg twice daily on Days 1-6, and a single dose on Day 7; with food; Panel B). Subject 106-102 in Cohort A had missed doses on Day 4 and Day 5 and was excluded for mean concentration calculation.

FIGS. 8A and 8B are a pair of graphs showing the individual and mean plasma concentration-time profiles after oral administration of multiple doses of Compound I to patients in MAD Cohort B (50 mg twice daily on Days 1-6, and a single dose on Day 7; with food; Panel A) and Cohort D (100 mg twice daily on Days 1-6, and a single dose on Day 7; with food; Panel B). Subject 401-101 in Cohort B had missed doses on Days 1-6 and was excluded for mean concentration calculation.

FIGS. 9A-9C are graphs showing the ECSG change from baseline by Compound I plasma concentration (9A), the SET change from baseline by Compound I plasma concentration (9B), and the change from baseline in LVSV by SET change from baseline (9C). The lines shown in FIGS. 9A and 9B are from a non-parametric LOESS (locally estimated scatterplot smoothing) method. The line shown in FIG. 9C, bound by the 95% upper and lower confidence limits, was generated from a mixed model regression accounting for within patient variation due to multiple measures taken from the same patient. Estimate of the slope is 0.1972 (p value<0.0001) with a 95% CI of (0.1479, 0.2465).

FIG. 10 is a set of graphs showing predicted and observed plasma concentration-time profiles for oral (PO) doses of 3 mg (top left), 100 mg (top right), and 525 mg (bottom left), as well as predicted in vivo absorption of Compound I at doses of 3, 100, and 525 mg in different regions of the gastrointestinal (GI) tract (bottom right). HV=healthy volunteers.

FIG. 11 is a set of graphs showing simulated in vivo dissolution (top right), absorption (bottom left), and plasma concentration-time (bottom right) profiles in healthy volunteers administered with 100 mg Compound I with different particle sizes. Also shown is predicted in vivo absorption of Compound I with different particle sizes in different regions of the GI tract (top left).

FIG. 12 is a set of graphs showing the effect of Compound I particle size on in vivo absorption and systemic exposure of Compound I administered at doses of 50, 100, 200, and 500 mg.

FIG. 13 is a table summarizing the data of the predicted and observed systemic exposure parameters following administration of Compound I to dogs.

FIG. 14 is a table summarizing the data of the predicted and observed systemic exposure parameters following administration of Compound I to healthy volunteers.

FIG. 15 is a schematic diagram showing the clinical trial design for treating primary DCM with documented MYH7 mutation with Compound I.

### DETAILED DESCRIPTION OF THE INVENTION

The present disclosure provides methods, uses, and compositions relating to treating systolic dysfunction (impairment of the systolic function of the heart; e.g., systolic heart failure) with the small molecule compound Compound I. The treatment regimens have been found to be safe and effective, leading to significant improvement of the cardiac functions of a treated patient.

### Pharmaceutical Compositions

The pharmaceutical compositions used in the present treatment regimens contain Compound I as an active pharmaceutical ingredient (API). Compound I refers to the compound (R)-4-(1-((3-(difluoromethyl)-1-methyl-1H-pyrazol-4-yl)sulfonyl)-1-fluoroethyl)-N-(isoxazol-3-yl) piperidine-1-carboxamide, which has the following chemical structural formula (I):

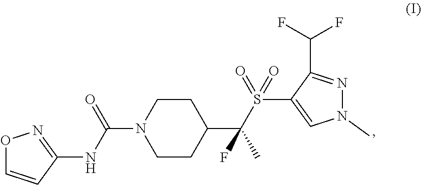 [SMILES: Cn1cc(S(=O)(=O)[C@@](C)(F)C2CCN(C(=O)Nc3ccon3)CC2)c(C(F)F)n1]

or a pharmaceutically acceptable salt thereof. Compound I is a myosin modulator that increases crossbridge formation (measured as phosphate release) between cardiac actin and myosin. Crossbridge formation and detachment are critical steps in each cycle of cardiac contraction. Compound I reversibly binds to myosin, increasing the number of myosin/actin crossbridges available to participate in the strongly bound state of the chemomechanical cycle and thereby increasing contraction. However, Compound I does not inhibit crossbridge detachment (measured as ADP release) and therefore does not affect any other states of the contraction cycle, nor does it affect calcium homeostasis.

The pharmaceutical compositions used herein may be provided in an oral dosage form (e.g., a liquid, a suspension, an emulsion, a capsule, or a tablet). In some embodiments, Compound I particles are compressed into tablets each containing 5, 25, 50, 75, 100, 125, 150, 175, or 200 mg of Compound I. In some embodiments, Compound I particles may be suspended in a suitable liquid such as water, a suspending vehicle, and/or flavored syrup for oral administration.

The Compound I API solid in the tablets or oral suspensions may have a mean particle size of, for example, 1-100, 1-50, or 15-50 μm in diameter (e.g., 1-5, 5-10, 1-10, 10-20, or 15-25 μm in diameter). In some embodiments, the Compound I has a mean particle size of no greater than 30, 25, 20, 15, 10, or 5 μm in diameter. In some embodiments, the Compound I API solid has a mean particle size of 15-25 μm in diameter for a particle size distribution (PSD) of D50 (i.e., 50% of the particles have a particle size of 15-25 μm in diameter). In certain embodiments, the Compound I has a mean particle size of 10 μm or less in diameter, e.g., D50 not more than (NMT) 10 μm. In certain embodiments, the Compound I has a mean particle size of 5 μm or less in diameter, e.g., D50 NMT 5 μm. The analysis of the particle size is typically carried out using a PSD method that is appropriate for determining the particle size of the primary particles. Ultrasound may be used to reduce agglomerates. The PSD technique used to measure particle size should not itself result in alteration of the primary particle size. In some of the Examples of the present disclosure, the PSD technique was performed with the Malvern Mastersizer 2000 with and without ultrasound.

Besides the Compound I API, the pharmaceutical compositions of the present disclosure may also contain pharmaceutically acceptable excipients. For example, the tablets used herein may contain bulking agents, diluents, binders, glidants, lubricants, and disintegrants. In some embodiments, Compound I tablets contain one or more of microcrystalline cellulose, lactose monohydrate, hypromellose, croscarmellose sodium, and magnesium stearate. The tablets may be coated to make them easier to ingest.

### Treatment Regimens

The safe and effective treatment regimens of the present disclosure were developed based on the results from clinical studies of Compound I in patients with systolic dysfunction. The Compound I treatment regimens increase myocardial contractility in a patient in need thereof while having no severe adverse effects on the ventricular diastolic functions of the patient (i.e., preserving relaxation). The patient may receive a treatment regimen of the present disclosure for at least one month, at least six months, at least twelve months, at least one year, or longer, or until such time the patient no longer needs the treatment.

In some embodiments of the present treatment regimens, Compound I is administered in a total daily oral amount of 10-700 mg (e.g., 25-700 or 50-150 mg). For example, Compound I may be administered in a total daily oral amount of 10, 25, 50, 75, 100, 125, 150, 175, 200, 250, 300, 350, 400, 450, 500, 525, 550, 600, or 700 mg. As another example, Compound I may be administered in a total daily oral amount of 50, 100, or 150 mg. In one embodiment, Compound I is orally administered at 10-175 mg (e.g., 25-175 mg) BID (twice daily) (e.g., 10, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170 or 175 mg). For example, Compound I may be orally administered at 10-75 or 25-75 mg (e.g., 10 mg, 25 mg, 50 mg, or 75 mg) BID (twice daily). In another embodiment, Compound I is orally administered at 25-350 mg QD (once daily) (e.g., 25-325, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, or 350 mg). The intervals between BID doses are, for example, between approximately 10-12 hours apart when possible (e.g., morning and evening). As used herein, administration of Compound I or a pharmaceutical composition containing Compound I (“Compound I medication”) includes self-administration by the patient himself or herself (e.g., oral intake by the patient). The Compound I medication may be taken by the patient at the indicated dosage, with or without food. The medication may be taken with a glass of drink such as water or milk (e.g., whole milk) if desired.

In some embodiments, a patient orally consumes a loading dose of Compound I with or without food followed by a maintenance dose (e.g., a dose described above) approximately 10-12 hours thereafter with or without food, and then continues his/her daily recommended maintenance dose regimen with or without food (e.g., morning and evening for BID dosing regimens). In one embodiment, for a targeted steady state mean concentration of 2000 ng/ml to 4000 ng/mL (e.g., 2000 ng/ml to 3500 ng/mL), the patient is administered with or without food (a) a loading dose of 2-fold the maintenance dose for a BID dosing regimen or 1.5-fold the maintenance dose for a QD dosing regimen, and (b) approximately 10-12 hours later, beginning the daily recommended BID or QD dosing regimen, whichever is applicable. In yet a further embodiment, a loading dose of 50-250 mg of Compound I is administered with or without food in the morning followed by a BID maintenance dosing regimen of 10-75 mg (e.g., 25-75 mg) BID or a QD maintenance dosing regimen of 75-125 mg QD beginning in the evening. A regimen comprising a twice-daily maintenance dose of 10-175 mg (e.g., 25-175 mg) with or without food, for example, could comprise the steps of (i) administering to the patient a loading dose of 2 times the maintenance dose, with or without food, and (ii) approximately 10-12 hours later, beginning the twice daily maintenance dosing regimen with or without food. A regimen comprising a once-daily maintenance dose of 25-350 mg with or without food, for example, could comprise the steps of (i) administering to the patient a loading dose of 1.5 times the maintenance dose, with or without food; and (ii) approximately 10-12 hours later, beginning the once daily maintenance dosing regimen with or without food.

In some embodiments, Compound I absorption by the patient may be facilitated by food. In some embodiments, the food is high in fat content; that is, more than 50% of the calories of the food are derived from fat). In some embodiments, where Compound I is taken with food (e.g., high fat food), the mean particle size of the Compound I API is over 15 μm in diameter and the QD dose is greater than approximately 200 mg. In some embodiments, the total daily dose of Compound I needed by a patient if the medication is taken in a fed state (e.g., within about two hours of food, within about one and a half hours of food, or within about one hour of food) may be lower than the total daily dose needed by the patient if the medication is taken not in a fed state. “Within about X hours of food” means about X hours before the start or after the end of ingestion of food.

In certain embodiments, Compound I tablets or capsules are taken orally by the patient—with food or within about two hours of food (e.g., within about one and a half hours of food or within about one hour of food)—twice a day; in further related embodiments, the Compound I medication contains Compound I particles having a mean particle size of D50 15-25 μm in diameter. In some embodiments, the patient takes the medication orally once daily with meals (e.g., 400-1000 calories, 25-50% fat). In some embodiments, the patient takes the medication twice daily with meals (e.g., 400-1000 calories per meal, 25-50% fat). For example, the patient may take the medication at breakfast and dinner.

In some embodiments, the Compound I API in the medication is micronized and has a mean particle size of 10 μm or less in diameter (D50 not more than (NMT) 10 μm), or of 5 μm or less in diameter (D50 NMT 5 μm). In certain embodiments, when Compound I particles in the medication have D50 NMT 5 or 10 μm, the medication may be taken orally by a patient twice a day (e.g., every 10-12 hours, or morning and evening), with or without food.

The dosage used for a particular patient may be adjusted based on the patient's condition and/or the patient's unique PK profile. Current studies indicate that the drug dosages and exposures tested are safe and are well tolerated. In some embodiments, Compound I may be administered to the patient at a dose that results in plasma concentrations of 1000 to 8000 ng/mL (e.g., 1000-2000 ng/mL, 1500-3000 ng/mL, 2000-3000 ng/mL, 3000-4000 ng/ml, 3000-4500 ng/ml, 3500-5000 ng/mL, 4000-5000 ng/mL, 5000-6000 ng/mL, 6000-7000 ng/mL, or 7000-8000 ng/mL). In some embodiments, Compound I may be administered to the patient at a dose that results in plasma concentrations of <2000, 2000-3500, or ≥3500 ng/mL (e.g., 2000-3500 ng/mL). In some embodiments, Compound I may be administered to the patient in amounts that result in a plasma Compound I concentration of greater than 1500, 2000, 2250, 2500, 2750, 3000, 3500, 4000, 5000, 6000, or 7000 ng/mL. In some embodiments, the Compound I target plasma concentration is between 1000-4000 ng/mL. In certain embodiments, the Compound I target plasma concentration is between 1500-3000 ng/mL. In particular embodiments, the Compound I target plasma concentration is between 2000-3500 ng/mL. The Compound I plasma concentration may be determined by any method known in the art, such as, for example, high performance liquid chromatography (HPLC), liquid chromatography-mass spectroscopy (LC-MS such as high performance LC-MS), gas chromatography (GC), or any combination thereof.

Well known pharmacokinetic (PK) parameters can be used to determine or adjust the dosing of Compound I in patients. The following are examples of PK parameters.

TABLE 1
PK Parameters

| Parameter | Definition |
|---|---|
| AUC | Area under the plasma concentration time curve |
| AUC₀₋ₜ | Area under the plasma concentration-time curve from time 0 to the last measurable |
| | concentration (tₗₐₛₜ) |
| AUC₀₋₂₄ | Area under the plasma concentration-time curve from time 0 to 24 hours |
| AUC₀₋₄₈ | Area under the plasma concentration-time curve from time 0 to 48 hours |
| AUC_{0-∞} | Area under the plasma concentration-time curve from time 0 to infinity. AUC_{0-∞} was |
| | calculated as the sum of AUC₀₋ₜ plus the ratio of the last measurable plasma |
| | concentration to the elimination rate constant. |
| Cₘₐₓ | Maximum observed measured plasma concentration over time span specified |
| C_{trough} | Trough plasma concentration at end of dosing interval |
| t_{1/2} | Apparent first-order terminal elimination half-life |
| t_{1/2}_{_{—}}_{λz} | Apparent terminal phase-phase elimination half-life |
| tₘₐₓ | Time of occurrence of Cₘₐₓ. If the maximum value occurred at more than 1 timepoint, |
| | tₘₐₓ was defined as the first timepoint with this value |
| t_{lag} | Time delay between drug administration and last time point prior to first nonzero |
| | concentration |
| V_{z}/F | Apparent volume of distribution uncorrected for bioavailability |
| CL/F | Apparent oral clearance |
| Mean residence | The average amount of time a drug remains in a compartment or system |
| time (MRT) | |
| Ae₀₋₂₄ | Amount of Compound I excreted in the urine from 0 to 24 hours after dosing |
| Ae₀₋₄₈ | Amount of Compound I excreted in the urine from 0 to 48 hours after dosing |
| % Dose₂₄ | Percent administered dose recovered in urine over 24 hours collection period |
| % Dose₄₈ | Percent administered dose recovered in urine over 48 hours collection period |
| CLᵣ | Renal clearance |

In some embodiments, the treatment regimens described herein comprise monitoring the patient for an adverse event such as headache, lethargy, chest discomfort, bradycardia, heart block, sinus tachycardia, ventricular tachycardia, palpitation, increase in NT-proBNP levels, increase in troponin levels, and cardiac ischemia. If a severe adverse event occurs, the patient may be treated for the adverse event, and/or may discontinue treatment with Compound I.

### Combination Therapy

The present disclosure provides both Compound I monotherapy and combination therapy. In combination therapy, a Compound I regimen of the present disclosure is used in combination with an additional therapy regimen, e.g., a guideline-directed medical therapy (GDMT), also referred to as a standard of care (SOC) therapy, for the patient's cardiac condition or other therapy useful for treating the relevant disease or disorder. The additional therapeutic agent may be administered by a route and in an amount commonly used for said agent or at a reduced amount, and may be administered simultaneously, sequentially, or concurrently with Compound I.

In certain embodiments, Compound I is administered on top of the SOC for a condition of systolic dysfunction, such as systolic heart failure. In some embodiments, the patient is given, in addition to the Compound I medication, another therapeutic agent such as a beta-blocker (e.g., bisoprolol, carvedilol, carvedilol CR, or metoprolol succinate extended release (metoprolol CR/XL)), an angiotensin converting enzyme (ACE) inhibitor (e.g., captopril, enalapril, fosinopril, lisinopril, perindopril, quinapril, ramipril, and trandolapril), an angiotensin receptor antagonist (e.g., an angiotensin II receptor blocker), an angiotensin receptor neprilysin inhibitor (ARNI) (e.g., sacubitril/valsartan), a mineralocorticoid receptor antagonist (e.g., an aldosterone inhibitor such as a potassium-sparing diuretic such as eplerenone, spironolactone, or canrenone), a cholesterol lowering drug (e.g., a statin), an I_{f} channel inhibitor (e.g., ivabradine), a neutral endopeptidase inhibitor (NEPi), a positive inotropic agent (e.g., digoxin, pimobendan, a beta adrenergic receptor agonist such as dobutamine, a phosphodiesterase (PDE)-3 inhibitor such as milrinone, or a calcium-sensitizing agent such as levosimendan), potassium or magnesium, a proprotein convertase subtilisin kexin-type 9 (PCSK9) inhibitor, a vasodilator (e.g., a calcium channel blocker, phosphodiesterase inhibitor, endothelin receptor antagonist, renin inhibitor, smooth muscle myosin modulator, isosorbide dinitrate, and/or hydralazine), a diuretic (e.g., a loop diuretic such as furosemide), a RAAS inhibitor, a soluble guanylate cyclase (sGC) activator or modulator (e.g., vericiguat), an SGLT2 inhibitor (e.g., dapagliflozin), an antiarrhythmic medication (e.g. amiodarone, dofetilide, and sotalol), an anticoagulant (e.g., warfarin, apixaban, rivaroxaban, and dabigatran), an antithrombotic agent, an antiplatelet agent, or any combination thereof.

Suitable ARBs may include, e.g., A-81988, A-81282, BIBR-363, BIBS39, BIBS-222, BMS-180560, BMS-184698, candesartan, candesartan cilexetil, CGP-38560A, CGP-48369, CGP-49870, CGP-63170, CI-996, CV-11194, DA-2079, DE-3489, DMP-811, DuP-167, DuP-532, E-4177, elisartan, EMD-66397, EMD-73495, eprosartan, EXP-063, EXP-929, EXP-3174, EXP-6155, EXP-6803, EXP-7711, EXP-9270, FK-739, GA-0056, HN-65021, HR-720, ICI-D6888, ICI-D7155, ICI-D8731, irbesartan, isoteoline, KRI-1177, KT3-671, KW-3433, losartan, LR-B/057, L-158809, L-158978, L-159282, L-159874, L-161177, L-162154, L-163017, L-159689, L-162234, L-162441, L-163007, LR-B/081, LR B087, LY-285434, LY-302289, LY-315995, LY-235656, LY-301875, ME-3221, olmesartan, PD-150304, PD-123177, PD-123319, RG-13647, RWJ-38970, RWJ-46458, saralasin acetate, S-8307, S-8308, SC-52458, saprisartan, saralasin, sarmesin, SL-91.0102, tasosartan, telmisartan, UP-269-6, U-96849, U-97018, UP-275-22, WAY-126227, WK-1492.2K, YM-31472, WK-1360, X-6803, valsartan, XH-148, XR-510, YM-358, ZD-6888, ZD-7155, ZD-8731, and zolasartan.

In particular embodiments, the additional therapeutic agent may be an ARNI such as sacubitril/valsartan (Entresto®) or a sodium-glucose cotransporter 2 inhibitor (SGLT2i) such as empaglifozin (e.g., Jardiance®), dapagliflozin (e.g., Farxiga®), canagliflozin (e.g., Invokana®), or sotagliflozin.

In some embodiments, a patient being treated for heart failure with Compound I is also being treated with an ARNI, a beta blocker, and/or an MRA.

In some embodiments, a patient being treated for heart failure with Compound I is also being treated with an ACE inhibitor and/or ARB and/or ARNI, in conjunction with a beta blocker and optionally an aldosterone antagonist. In certain embodiments, the ACE inhibitor, ARB, ARNI, beta blocker, and/or aldosterone antagonist are selected from those described herein, in any combination.

If any adverse effect occurs, the patient may be treated for the adverse effect. For example, a patient experiencing headache due to the Compound I treatment may be treated with an analgesic such as ibuprofen and acetaminophen. A patient experiencing arrhythmia due to the Compound I treatment may be treated with antiarrhythmic drugs such as amiodarone, dofetilide, sotalol, flecainide, ibutilide, lidocaine, procainamide, propafenone, quinidine, and tocainide.

### Patient Populations

The treatment regimens of the present disclosure may be used to treat a patient exhibiting systolic dysfunction such as systolic heart failure. Systolic heart failure may be characterized by reduced ejection fraction (e.g., less than about 50%, 45%, 40%, or 35%, including LVEF of 15-35%, 15-40% (e.g., 15-39%), 20-45%, 40-49%, and 41-49%) and/or increased ventricular end-diastolic pressure and volume. In some embodiments, the systolic heart failure is HFrEF (ejection fraction of <50%, e.g., ≤40% or <40%).

A treatment regimen herein may include the step of selecting a patient with a type of systolic heart failure as described herein. In some embodiments, the patient is 18 years of age or older. In some embodiments, the patient has never been treated for HF. In some embodiments, the patient has previously been or is being treated for HF, such as systolic heart failure, with, for example, the standard of care for HF, but has not shown adequate improvement. In some embodiments, the patient has been or is being treated with Entresto® and/or omecamtiv but continues to exhibit systolic heart failure symptoms. In some embodiments, the patient has been or is being treated with an ACE inhibitor or an ARB or an ARNI in conjunction with a beta blocker and optionally an aldosterone antagonist (wherein these agents may be, e.g., selected from those described herein), but continues to exhibit systolic heart failure symptoms. The patient may have chronic HF, i.e., having systolic heart failure for four weeks or more while receiving the standard of care for HF; or the patient may have recent HF, i.e., having systolic heart failure for less than four weeks while receiving the standard of care for HF. If a patient experiences symptoms that appear suddenly (e.g., congestion symptoms such as shortness of breath) that lead to hospital admission, or a rapid worsening of existing symptoms of heart failure, this is often referred to as acute HF.

The patient may experience systolic heart failure of the left ventricle, the right ventricle, or both ventricles. In some embodiments, the patient has right ventricular heart failure. In further related embodiments, the patient has pulmonary hypertension (i.e., pulmonary arterial hypertension).

In some embodiments, the patient has HFrEF (i.e., an ejection fraction of <50%). HFrEF with an ejection fraction of ≤40% is classical HFrEF, while HFrEF with an ejection fraction of 41-49% is classified as heart failure with mid-range ejection fraction (HFmrEF). The patient may have a reduced left ventricular ejection fraction (LVEF) of less than 50%, e.g., less than 45%, 40%, 35%, 30%, 25%, 20%, or 15%. In certain embodiments, the patient has L VEF≤45% (e.g., 20-45%), ≤40% (e.g., 15-40%, 25-40%, 15-39%, or 25-39%), or ≤35% (e.g., 15-35%). The HFrEF may be of ischemic or non-ischemic origin, and may be chronic or acute.

In particular embodiments, the patient has high-risk HFrEF (or “higher-risk HFrEF” as used herein). High-risk HFrEF patients are patients who have an LVEF of 35% or less. In some embodiments, the patient is further diagnosed with NYHA Class III or IV. In some embodiments, the patient has an LVEF of 30% or less. In some embodiments, a HFrEF patient is further considered “high-risk” when he/she meets one or more of the following criteria:
(i) frequent hospitalizations for worsening heart failure (WHF);
(ii) hospitalization for WHF despite being on a high dose of diuretic;
(iii) LVEF <30% or <35%;
(iv) elevated N-terminal pro b-type natriuretic peptide NT-proBNP (e.g., ≥400, 600, 800, 1000, or 1200 μg/mL);
(v) heavy symptom burden (NYHA Class III-IV, infra);
(vi) low functional or exercise capacity (as determined by, for example, peak VO₂, 6-min walk-test, and/or activity (as determined by, e.g., accelerometry));
(vii) IV inotrope-dependent; and
(viii) inability to be treated with recommended (guideline-directed) HF medications at optimal doses (e.g., a RAAS inhibitor such as an angiotensin converting enzyme (ACE) inhibitor, an angiotensin receptor blocker (ARB), an ARNI (e.g., Entresto®), a beta blocker, a mineralcorticoid receptor antagonist (MRA), etc.).

In further embodiments, a HFrEF patient is considered “high-risk” when he/she meets the following criteria:
(a) NYHA Class III-IV;
(b) LVEF≤35%; and
(c) elevated NT-proBNP of ≥400, 600, 800, 1000, or 1200 μg/mL.

In some embodiments, the patient has stable HF, e.g., stable HFrEF. As used herein, a patient who is “stable” with regard to a disease refers to a patient who has the disease and is not experiencing worsening of symptoms that might lead to a hospitalization or an urgent visit. For example, patients with stable HF can have impaired systolic function, but the symptoms of the dysfunction can be controlled or stabilized using available therapies.

In some embodiments, the patient has stable HFrEF (e.g., stable, chronic HFrEF of moderate severity), as defined by one or both of the following: (i) LVEF of less than 50%; and (ii) chronic medication for treatment of heart failure consistent with current guidelines, which may include at least one of beta-blocker, ACE inhibitor, ARB, and ARNI. In certain embodiments, the patient does not have any one or combination of:
(a) current angina pectoris;
(b) recent (<90 days) acute coronary syndrome;
(c) coronary revascularization (percutaneous coronary intervention (PCI) or coronary artery bypass graft (CABG)) within the prior 3 months; and
(d) uncorrected severe valvular disease.

In some embodiments, the patient further has an L VEF less than 40% or 35%, between 15% and 40%, or between 15% and 35%. In some embodiments, the patient further has NT-proBNP levels greater than 400 μg/mL.

In some embodiments, the treatment regimens of the present disclosure may be used to treat a patient exhibiting dilated cardiomyopathy (DCM) (e.g., idiopathic DCM or genetic DCM). In certain embodiments, the patient has a dilated left or right ventricle, an ejection fraction less than 50% (e.g., ≤40%), and no known coronary disease. The DCM may be genetic DCM, wherein the patient has at least one genetic mutation in a sarcomeric contractile or structural protein that is known to cause DCM (see, e.g., Hershberger et al., Nat Rev Cardiol. (2013) 10 (9): 531-47 and Rosenbaum, supra), such as myosin heavy chain, titin, or troponin T. In some embodiments, the genetic mutation is in a gene selected from ABCC9, ACTC1, ACTN2, ANKRD1, BAG3, CRYAB, CSRP3, DES, DMD, DSG2, EYA4, GATAD1, LAMA4, LDB3, LMNA, MYBPC3, MYH6, MYH7, MYPN, PLN, PSEN1, PSEN2, RBM20, SCN5A, SGCD, TAZ, TCAP, TMPO, TNNC1, TNNI3, TNNT2, TPM1, TTN, VCL, or any combination thereof. For example, the genetic mutation is in a gene selected from ACTC1, DES, MYH6, MYH7, INNC1, INNI3, TNNT2, TTN, or any combination thereof. In particular embodiments, the genetic mutation is in the MYH7 gene. In certain embodiments, the patient with DCM (e.g., genetic DCM, which may be caused by a mutation in the MYH7 gene) also has HFrEF, and may exhibit one or more (e.g., all) of the following:
has an LVEF of 15-40%;
has at least mild left ventricular enlargement (LVEDD≥3.1 cm/m² for males, ≥3.2 cm/m² for females); and
receives chronic medication for the treatment of heart failure, such as a β-blocker, angiotensin converting enzyme (ACE) inhibitor, angiotensin receptor blocker (ARB), angiotensin receptor neprilysin inhibitor (ARNI), or any combination thereof.

In certain embodiments, the patient does not exhibit one or more (e.g., all) of the following:
a QTcF interval >480 msec;
where the genetic mutation is in the MYH7 gene, known pathogenic mutation of another gene implicated in DCM;
HFrEF that is considered to be caused primarily by ischemic heart disease, chronic valvulopathy, or another condition;
recent (<90 days) acute coronary syndrome or angina pectoris;
coronary revascularization (percutaneous coronary intervention [PCI] or coronary artery bypass graft [CABG]) within prior 90 days;
recent (<90 days) hospitalization for heart failure, use of IV diuretic or chronic IV inotropic therapy or other cardiovascular event (e.g., cerebrovascular accident); and
known aortic stenosis of moderate or greater severity.

In some embodiments, the patient treated with a treatment regimen described herein has New York Heart Association (NYHA) Class I, II, III, or IV heart failure, as defined in Table 2 below.

TABLE 2
New York Heart Association (NYHA) Classes of Heart Failure

| Class | Patient Symptoms |
|---|---|
| I | No limitation of physical activity. Ordinary physical |
| | activity does not cause undue fatigue, palpitation, |
| | dyspnea (shortness of breath). |
| II | Slight limitation of physical activity. Comfortable at |
| | rest. Ordinary physical activity results in fatigue, |
| | palpitation, dyspnea (shortness of breath). |
| III | Marked limitation of physical activity. Comfortable at |
| | rest. Less than ordinary activity causes fatigue, |
| | palpitation, or dyspnea. |
| IV | Unable to carry on any physical activity without |
| | discomfort. Symptoms of heart failure at rest. If any |
| | physical activity is undertaken, discomfort increases. |

Additional or concomitant conditions that can be treated by the treatment regimens of the present disclosure include, without limitation, HFpEF, chronic congestive heart failure, cardiogenic shock and inotropic support after cardiac surgery, hypertrophic cardiomyopathy, ischemic or post-infarction cardiomyopathy, viral cardiomyopathy or myocarditis, toxic cardiomyopathies (e.g., post-anthracycline anticancer therapy), metabolic cardiomyopathies (in conjunction with enzyme replacement therapy), diabetic cardiomyopathy, diastolic heart failure (with diminished systolic reserve), atherosclerosis, secondary aldosteronism, and ventricular dysfunction due to on-bypass cardiovascular surgery. A treatment regimen of the present disclosure may also promote salutary ventricular reverse remodeling of left ventricular dysfunction due to ischemia or volume or pressure overload, e.g., myocardial infarctions, chronic mitral regurgitation, chronic aortic stenosis, or chronic systemic hypertension, and/or treat detrimental vascular remodeling. By reducing left ventricular filling pressures, the treatment regimens could improve the symptom of dyspnea and reduce the risk of pulmonary edema and respiratory failure. The treatment regimens may reduce the severity of the chronic ischemic state associated with DCM and thereby reduce the risk of Sudden Cardiac Death (SCD) or its equivalent in patients with implantable cardioverter-defibrillators (frequent and/or repeated ICD discharges) and/or the need for potentially toxic antiarrhythmic medications. The treatment regimens could be valuable in reducing or eliminating the need for concomitant medications with their attendant potential toxicities, drug-drug interactions, and/or side effects. The treatment regimens may reduce interstitial myocardial fibrosis and/or slow the progression of, arrest, or reverse left ventricular stiffness and dysfunction.

In some embodiments, the treatment regimens of the present disclosure may be used to treat a patient with heart failure (e.g., HFrEF) who exhibits mitral regurgitation. In some embodiments, the mitral regurgitation is chronic. In some embodiments, the mitral regurgitation is acute.

In some embodiments, patients with systolic dysfunction may display increased levels of biomarkers in the blood. Circulating natriuretic peptide (NP) levels add incremental prognostic value to standard clinical risk stratification algorithms for both ambulatory and hospitalized heart failure patients, with a steady increase in the risk of mortality and recurrent heart failure hospitalization as NT-proBNP levels rise above 1000 pg/m. See, e.g., Desai et al., Circulation (2013) 127:509-516. For example, brain natriuretic peptide (BNP) or N-terminal-pro-brain natriuretic peptide (NT-proBNP) is present at elevated levels in the blood of individuals with systolic dysfunction. A normal level of BNP is less than 100 μg/mL. The higher the number, the more likely heart failure is present and the more severe it is likely to be. A normal level of NT-proBNP, based on Cleveland Clinic's reference range is: (1) less than 125 μg/mL for patients aged 0-74 years, and (2) less than 450 μg/mL for patients aged 75-99 years.

Accordingly, in some embodiments, a patient to be treated with a treatment regimen of the present disclosure may exhibit elevated serum blood levels of brain natriuretic peptide (BNP) or N-terminal-pro-brain natriuretic peptide (NT-proBNP). In some embodiments, a patient's serum blood level of BNP is considered elevated when the concentration is at least 35, 45, 55, 65, 75, 85, 95, 100, 105, or 115 μg/mL (for example, at least 35 or 85 μg/mL). In some embodiments, a patient's serum blood level of NT-proBNP is considered elevated when the concentration is at least 95, 105, 115, 125, 135, 145, 155, 165, or 175 μg/mL (for example, at least 125 or 155 μg/mL).

In some embodiments, the patient may not receive (temporarily or permanently), or may discontinue, Compound I treatment if he/she has one or more of the following conditions:
(i) acute coronary syndrome (ACS);
(ii) stroke;
(iii) major cardiac surgery/intervention;
(iv) coronary intervention;
(v) cardiac valve repair/implantation within three months;
(vi) uncorrected valvular or clinically significant congenital heart disease;
(vii) mechanical support≤7 days;
(viii) planned LVAD or transplant within 60 days; and
(ix) IV inotrope dependent.

### Treatment Outcomes

As used herein, the terms “treat,” “treating” and “treatment” refer to any indicia of success in the treatment or amelioration of a pathology, injury, condition, or symptom related to systolic dysfunction, including any objective or subjective parameter such as abatement; remission; diminishing of symptoms; making the pathology, injury, condition, or symptom more tolerable to the patient; decreasing the frequency or duration of the pathology, injury, condition, or symptom; or, in some situations, preventing the onset of the pathology, injury, condition, or symptom. Treatment or amelioration can be based on any objective or subjective parameter; including, e.g., the result of a physical examination. For example, treatment of systolic heart failure encompasses, but is not limited to, improving the cardiac functions of the patient and alleviating the of symptoms of systolic heart failure (especially during exercise, including walking or stair climbing). Symptoms of systolic heart failure may include, e.g., excessive fatigue, sudden weight gain, a loss of appetite, persistent coughing, irregular pulse, chest discomfort, angina, heart palpitations, edema (e.g., swelling of the lungs, limbs, face, or abdomen), dyspnea, protruding neck veins, and decreased exercise tolerance and/or exercise capacity.

Pharmacodynamic (PD) parameters that can be used to measure the cardiac functions of a patient are shown in Table 3 below. These PD parameters are routinely used by clinicians and can be measured by standard transthoracic echocardiogram, as illustrated in the Working Examples below.

TABLE 3
Transthoracic Echocardiography (TTE) Parameters

| | |
|---|---|
| Abbreviation | Parameter |

Direct measures of contractility/systolic function

| | |
|---|---|
| CO | Cardiac Output |
| LVOT-VTI | Left ventricular outflow tract - velocity time integral |
| LVESD | Left ventricular end-systolic diameter |
| LVGLS | Left ventricular global longitudinal strain |
| LVGCS | Left ventricular global circumferential strain |
| PEP | Pre-ejection period |
| IVCT | Isovolumic (isovolumetric) contraction time |
| s′ (lateral) | Peak atrioventricular valve annular velocity in systole |

Indirect (derived) measures of contractility/systolic function

| | |
|---|---|
| LVEF | Left ventricular ejection fraction |
| LVFS | Left ventricular fractional shortening |
| LVESV | Left ventricular end-systolic volume |
| LVSV | Left ventricular stroke volume |
| MPI | Myocardial performance index |

Measures of ventricular relaxation/diastolic function

| | |
|---|---|
| LVEDD | Left ventricular end-diastolic diameter |
| LVEDV | Left ventricular end-diastolic volume |
| Peak E | Maximum mitral blood flow rate during early |
| | diastolic filling |
| Peak A | Maximum mitral blood flow rate during late |
| | diastolic filling |
| E/A ratio | Ratio of maximum mitral blood flow rate during |
| | early diastolic filling to flow rate during |
| | late diastolic filling |
| e′ (lateral) | Peak atrioventricular valve annular velocity in |
| | early diastole |
| E/e′ ratio | Ratio of E to e′ (mitral annular blood flow rate) |
| IVRT | Isovolumic (isovolumetric) relaxation time |

Measure of duration of systole

| | |
|---|---|
| SET | Systolic ejection time |

The present treatment regimens may lead to one or more of the improved left ventricular functions selected from improved cardiac contractility as indicated by increased stroke volume, increased cardiac output, increased ejection fraction, increased fractional shortening, improved global longitudinal strain, improved global circumferential strain and/or decreased left ventricular end-systolic or end-diastolic diameter, and with mild to moderate (e.g., modest) systolic ejection time (SET) prolongation. The regimens may result in improved symptoms as measured by improvement in NYHA Class and/or reduction of dyspnea. The regimens may result in improved functional and/or exercise capacity of the patient as measured by peak VO₂, 6-minute walk test, and/or activity (as determined by accelerometry). In particular embodiments, the present treatment regimens may lead to one or more of the following outcomes in a patient with systolic heart failure:
(i) improvement in one or more of LVEF, LVFS, LVSV, CO, GLS, GCS, E/A, and E/e′ (e.g., as measured by ECHO);
(ii) downgrade in NYHA Class;
(iii) reduced NT-proBNP levels;
(iv) improved exercise capacity as measured by peak VO₂, 6-minute walk test, and/or activity as determined by accelerometry); and
(v) improved patient-reported outcomes.

In some embodiments, the present treatment regimens result in one or more of the following:
(i) increase in L VEF and/or LVSV;
(ii) decrease in LVGLS, LVESV, and/or LVEDV; and
(iii) minimal impact on diastolic function and relaxation (as measured by direct measures such E, e′, E/e′, E/A, IVRT).

The present treatment regimens reduce the risk of cardiovascular death, and/or hospitalization/urgent care visits for HF in patients with systolic heart failure, patients with HFrEF (e.g., stable or high-risk HFrEF), patients with chronic heart failure (NYHA Class I-IV (e.g., Class II-IV) and reduced ejection fraction, or any other patient populations described above. By “reducing the risk” of an event is meant increasing the time to the event by at least 10% (e.g., at least 15%, 20%, 30%, 40%, 50%, 60%, 70%, 80%, or 90%).

In some embodiments, the present treatment regimens alleviate or prevent one or more symptoms of heart failure, which include, for example, dyspnea (e.g., orthopnea, paroxysmal nocturnal dyspnea), coughing, cardiac asthma, wheezing, hypotension, dizziness, confusion, cool extremities at rest, pulmonary congestion, chronic venous congestion, ankle swelling, peripheral edema or anasarca, nocturia, ascites, hepatomegaly, jaundice, coagulopathy, fatigue, exercise intolerance, jugular venous distension, pulmonary rales, peripheral edema, pulmonary vascular redistribution, interstitial edema, pleural effusions, fluid retention, or any combination thereof. Other signs and symptoms of HF that may be improved by a treatment regimen of the invention include, e.g., compensatory mechanisms characterized by increased sympathetic tone, peripheral vasoconstriction, activation of various neurohormonal pathways, sodium retention, arterial and venous constriction, neuroendocrine activation, and increased heart rate.

In some embodiments, the present treatment regimens result in reduction of the risk of cardiovascular death (e.g., by 10, 15, 20, 25, 30, 35, 40, 45, or 50%) and/or the frequency and/or duration of cardiovascular hospitalization.

In some embodiments, the present treatment regimens reduce urgent outpatient intervention for heart failure.

The advantages of the present treatment regimens include the features that the treatment
(i) has minimal impact on relaxation (e.g., no more than a modest increase in systolic ejection time and no discernable effect on diastolic function), calcium homeostasis, or troponin level (e.g., no more than a mild elevation of troponin);
(ii) does not impair ADP release;
(iii) does not change cardiac phase distribution;
(iv) has no more than a modest effect on SET;
(v) does not cause drug-related cardiac ischemia (e.g., as determined by clinical symptoms, ECG, cardiac biomarkers such as troponin, creatine kinase-muscle/brain (CK-MB), cardiac imaging, and coronary angiograms);
(vi) does not cause drug-related atrial or ventricular arrhythmia;
(vii) does not cause drug-induced liver injury as measured by alanine aminotransferase or aspartate aminotransferase, bilirubin; and
(viii) also does not result in abnormalities in the patient's urine, serum, blood, systolic blood pressure, diastolic blood pressure, pulse, body temperature, blood oxygen saturation, or electrocardiography (ECG) readings.

Diastolic dysfunction may also be associated with systolic heart failure, and can contribute to morbidity. By preserving relaxation, the present treatment regimens may lead to enhanced clinical benefits over treatments with cardiac myosin activators that do not preserve relaxation.

### Articles of Manufacture and Kits

The present invention also provides articles of manufacture, e.g., kits, comprising one or more dosages of the Compound I medication, and instructions for patients (e.g., for treatment in accordance with a method described herein). The articles of manufacture may also contain an additional therapeutic agent in the case of combination therapy. Compound I tablets or capsules may be blistered and then carded, produced with, for example, 5-20 tablets per blister card; each tablet or capsule may contain 5, 25, 50, 75, or 100 mg of Compound I, and such blister card may or may not additionally include a loading dose tablet or capsule. The present disclosure also includes methods for manufacturing said articles.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cardiology, medicine, medicinal and pharmaceutical chemistry, and cell biology described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. It should also be noted that the term “or” is generally employed in its sense including “and/or” unless the content clearly dictates otherwise. As used herein the term “about” refers to a numerical range that is 10%, 5%, or 1% plus or minus from a stated numerical value within the context of the particular usage. Further, headings provided herein are for convenience only and do not interpret the scope or meaning of the claimed embodiments.

All publications and other references mentioned herein are incorporated by reference in their entirety. Although a number of documents are cited herein, this citation does not constitute an admission that any of these documents forms part of the common general knowledge in the art.

In order that this invention may be better understood, the following examples are set forth. These examples are for purposes of illustration only and are not to be construed as limiting the scope of the invention in any manner.

### EXAMPLES

### Example 1: Randomized, Placebo-Controlled Study of Safety, Tolerability, Preliminary Pharmacokinetics and Pharmacodynamics of Single Ascending Oral Doses of Compound I in Healthy Adult Volunteers

This example describes the first-in-human study of Compound I. Based on its mechanism of action, Compound I may offer a targeted therapy for patients with DCM caused by genetic or nongenetic mechanisms. The study was a randomized, double-blind, placebo-controlled, sequential group, single-ascending (oral) dose study in healthy subjects aged 18-55 years. Eight dosing cohorts, each comprising eight healthy subjects, were enrolled. Within each cohort, subjects were randomized 6:2 to Compound I: placebo.

### Materials and Methods

### Study Design

Subjects were resident at the clinical site for up to 5 days and 4 nights, from Day-1 (the day before dosing) to Day 4, and received a single dose of Compound I or placebo on Day 1. ECG telemetry was initiated 1 hour predose and continued through 48 hours postdose (Day 3). Any subject with a predose resting HR ≥80 beats per minute was considered ineligible and not treated. If the half-life Compound I was significantly longer than the predicted 12 hours, the SRC could have modified the study timeline to confine subjects to the unit for PK sampling or PD measurements for a time period equivalent to about 5 times the mean terminal half-life, but no longer than 5 days after dosing. Subjects returned for a safety follow-up visit 7 days (+1 day) after dosing.

Because this was the first-in-human study, a sentinel dosing plan was employed at each dose level. The first 2 subjects of each cohort were dosed as sentinels. One of the sentinel subjects was randomized to receive Compound I and the other was randomized to receive placebo. Following review of safety data through 24 hours from the sentinel subjects, 1 or 2 subjects per day could have been enrolled. On each study day, the second subject was not dosed until after the time of the predicted peak plasma concentration (predicted tₘₐₓ) for the first subject had elapsed, and the investigator or subinvestigator reviewed the safety data, vital signs, and ECGs obtained from the first subject through the interval encompassing the predicted peak plasma concentrations of Compound I. Before each day of dosing, the investigator or the subinvestigator reviewed the safety data from the previous subjects including vital signs, safety laboratory values, hs-troponin I concentrations, and ECGs.

To assess pharmacodynamic effects, serial echocardiograms were performed. The sonographers used in the study completed Echo protocol training and submitted an example of a study for evaluation to the core laboratory for evaluation. The core TTE lab certified that the sonographer was able to perform the TTEs at a level satisfactory for obtaining the required protocol data.

Dose escalation stopping criteria included an increase in the mean maximal SET >50 msec in a cohort at any time point or if any subject had a prolongation of SET of ≥75 msec measured at any 2 sequential TTE assessments. These criteria were chosen to prevent subjects from having prolongation of SET that might lead to myocardial ischemia. Dose escalation stopping criteria also included observation of a Baseline-corrected, group mean relative increase of >20% in any 2 sequential TTE assessments in at least 2 measures of LV contractility: LVOT-VTI, LVFS, LVEF, or LVSV in subjects receiving Compound I. Placebo-controlled evaluation may have been considered. For this comparison, subjects who received placebo may have been pooled across cohorts.

After every dose level, the SRC conducted a blinded review of the data, but may have unblinded the data if there was a safety concern or they believed that possible PD changes were observed. The dosing information on 2 subjects was unblinded as described below.

### Treatment Administered

All randomized study subjects received either Compound I or matching placebo as a single oral dose after a fasting period of at least 6 hours. Compound I drug substance is a crystalline, free-base, synthetic molecule with a molecular weight of 435.4 g/mol. Compound I is nonhygroscopic and practically insoluble in aqueous media.

Compound I was provided as a powder for oral suspension. Placebo was provided as calcium carbonate powder. Both treatments were dosed orally as a suspension. The suspension was made using Ora-Plus® suspending vehicle (Perrigo) and a cherry syrup flavoring vehicle (Humco), mixed 50% to 50%. The suspension was followed by approximately 100 mL water. The suspension was made up within 14 days from the time in which it was dosed which was consistent with the stability data on the suspension. The suspension was made up so the volume administered to the subjects who received Compound I was the same at 20 mL.

### Dosing Escalation

The starting dose was set at 3 mg, using the FDA guidance of 60-kg weight for humans. After the first dose, dose escalation was approximately 3-fold until reaching a dose that was predicted to have a Cₘₐₓ of 300 ng/mL or where early PD activity was observed. Dose escalation thereafter was 2-fold. If the PK data were not consistent with the predicted PK profile, the dose escalation steps were to be no greater than 2-fold. Dose escalation was terminated using prospectively defined stopping criteria upon acquisition and was terminated based on 2 observations. The first was that the exposures were not increasing in a dose-proportional manner. It appeared that exposures at doses greater than 350 mg were no higher than the exposure after the administration of the 350 mg dose. In addition, the decision to stop dose escalation was also triggered when initial PD activity was observed after the administration of the 350 mg and the 525 mg (both with approximately the same exposure) allowed for initial estimate of the dose-response relationship of effect based on the PD parameters distinguishable from the placebo group.

Each subject received a dose according to the cohort in which they were enrolled. Cohorts were enrolled sequentially, with each cohort receiving an escalated dose of Compound I. The doses administered were 3 mg, 10 mg, 25 mg, 50 mg, 100 mg, 175 mg, 350 mg, and 525 mg, respectively.

### PK, PD, and Safety Assessment

PK and PD data were collected as described herein. (The exposure (both Cₘₐₓ and AUC) after the administration of the single dose of 350 mg and 525 mg was very similar, so data from the 2 groups were combined for some of the PD analyses. Safety was assessed throughout the study. Safety assessments included medical history, physical examinations, SET by TTE, 12-lead ECGs and ECG telemetry, vital signs, serum hs-troponin I concentrations, AEs, and safety laboratory results. SET determined by photoplethysmography was an exploratory safety parameter. Safety laboratory data including hematology, chemistry, and vital signs were evaluated by timepoint for the Safety Analysis Population using descriptive statistics. Changes from Baseline at each postbaseline timepoint were assessed.

### Medical History and Physical Examinations

A complete medical history was recorded at the Screening visit, which included evaluation (past or present) of the following: general, head and neck, eyes, ears, nose, throat, chest/respiratory, heart/cardiovascular, gastrointestinal/liver, gynecological/urogenital, musculoskeletal/extremities, skin, neurological/psychiatric, endocrine/metabolic, hematologic/lymphatic, allergies/drug sensitivities, past surgeries, substance abuse, or any other diseases or disorders as well as participation in clinical studies (study medication and/or device or other therapy). The medical history was updated at Day −1, if needed.

At Screening and Day −1, a complete physical examination was conducted including a neurological examination (gross motor and deep tendon reflexes), and assessment of the following: general appearance, skin, head and neck, mouth, lymph nodes, thyroid, abdomen, musculoskeletal, cardiovascular, neurological, and respiratory systems. At all other time points, an abbreviated physical examination (pulmonary, cardiac, abdominal, and other systems related to symptoms) was conducted

### Systolic Ejection Time

SET as determined by TTE was assessed using summary statistics. Observations and change from Baseline were summarized by treatment at each time point and the maximum change from Baseline determined for each subject. In addition, categorical analyses were performed on the number of subjects with a change from Baseline >50 msec and the number of subjects with a change from Baseline >75 msec in 1 or any 2 sequential TTE assessments. The relationship to Compound I plasma concentration to SET was explored. An analysis of SET placebo-adjusted change from Baseline was also performed.

An experimental noninvasive optical biosensor resembling a FitBit was fastened to the subject's wrist for several minutes during the conduct of each TTE to collect data on arterial pulse wave morphology by photoplethysmography.

### Electrocardiograms

A 12-lead electrocardiogram (ECG) was obtained after the subjects had rested in a supine position for at least 10 minutes. If the subject had a troponin-I abnormality or any signs or symptoms suggestive of possible cardiac ischemia, additional ECGs would be obtained. Digital 12-lead ECG evaluations was performed after 10 minutes of rest at Screening, predose on Day 1 (within 2 hours of dosing), and at various predetermined time points. Each time an ECG was completed, a 10-second paper ECG rhythm strip would also be obtained and maintained in the subject's source documentation.

The Investigator would judge the overall ECG interpretation as (a) normal, (b) abnormal without clinical significance, or (c) abnormal with clinical significance. If clinically significant, the abnormality would be recorded. In addition, before each treatment period, the Investigator or Subinvestigator would review the available ECGs from the previous treatment periods looking for signs of ischemia. If there were signs of ischemia, continued dosing would be withheld until there was full understanding of the possible ischemic changes.

The ECGs were transmitted to the core ECG laboratory who read the recordings in a blinded manner. An automated methodology was utilized with manual over-reading by a cardiologist. The following intervals were measured: RR, PR, QRS, and QT. Heart rate (HR) was calculated as 60/(RR×1000) (with RR expressed in msec) and rounded to the nearest integer.

### Correction for Heart Rate

Corrected QT interval (QTc) was calculated using the manually over-read QT values per the standard procedures of the central ECG laboratory. Each individual ECG QT value was corrected for HR. The measured QT data were corrected for HR using the Fridericia correction QTcF and the Bazzett method (QTcB) as per the following formulae/method (with QT, RR and QTc expressed in msec):

$$QTcX = \frac{QT}{{( RR / 1000 )}^{) 1 / n )}}$$

Fridericia, X=F, n=3; Bazzett, X=B, n=2.

### ECG Numeric Variables

HR, PR, QRS, and QTcF were summarized using descriptive statistics. The change from Baseline of these ECG parameters at each timepoint was listed for each subject. For each time point of measurement, the changes from Baseline were summarized using descriptive statistics. The relationship between HR/ECG intervals and time was plotted.

### Categorical Analysis

The incidence count and percentage of subjects with any postdose QTcF values of >450 msec, >480 msec, and >500 msec were tabulated for all subjects. Subjects with QTc values >500 msec were listed with corresponding Baseline values, ΔQTcF, and Baseline and treatment HR. The incidence count and percentage of subjects with ΔQTcF increases of >30 msec and >60 msec were tabulated.

### Morphology Findings

New ECG morphologies for each subject not present on any ECG at Baseline for that subject were summarized for all observation time points combined. The number and percentage of subjects having T wave morphology changes and/or the occurrence of abnormal U-waves that represent the appearance or worsening of the morphological abnormality from Baseline are reported.

### Concentration-QTc Analyses

A concentration-QTc regression analysis, based on data collected from the ECG recordings after study drug administration and drug plasma concentration values for each subject at each matching time point, was performed.

### Adverse Events

Any abnormal findings judged by the investigator to be clinically important were recorded as adverse events (AEs). AEs were mapped to system organ classes (SOCs) and preferred terms (PTs) using the Medical Dictionary for Regulatory Activities (MedDRA). AEs were monitored during the study and the data analyzed with respect to overall incidence, severity, and potential relationship to study medication. The blinded AEs were presented to the SRC for review after each cohort to aid in their decision on the dose of the subsequent cohort or if the study should be terminated. The study committee unblinded the data for one subject who had an arrhythmia TEAE and a second subject with mildly elevated hs-Troponin I levels (16 ng/mL, normal range 0 to 15 ng/mL) 6 hours postdosing and intermittent premature ventricular contractions (PVCs) on telemetry monitoring >48 hours after dosing. No ECG changes or symptoms were noted.

For the final analysis, the AEs were grouped by treatment group with all of the subjects who received placebo pooled as 1 group. AEs with onset on or after the first dose of study medication, or with an onset before the first dose of study medication that increased in severity on or after the first dose of study medication. Treatment-emergent AEs (defined as AEs starting from informed consent through the duration of the study) were summarized for the Safety Analysis Population by MedDRA SOC and PT, and by severity and relationship to treatment. Severe and life-threatening AEs, SAEs, and AEs leading to study withdrawal, if any, were presented in data listings.

Serum hs-Troponin I Concentrations

Serum samples were drawn for hs-troponin I. Analyses were performed using the Abbott Architect STAT High Sensitivity Troponin I assay. If a subject had any signs or symptoms suggestive of possible cardiac ischemia, additional serial hs-troponin I samples were obtained as appropriate to evaluate the possibility of ischemia.

### Drug Concentration Measurements

The concentrations of Compound I in human plasma and urine were quantitated by high performance liquid chromatography with tandem mass spectrometric detection (LC MS/MS) (Biological sample analysis study report Alturas AD17-726). Plasma samples were extracted by protein precipitation with acetonitrile containing internal standard MYK-5654. The calibration curves were linear over concentration range of 0.500 to 1000 ng/mL with a lower limit of quantification (LLOQ) of 0.500 ng/mL.

The PK Population included all subjects who received Compound I. Blood samples were collected for PK assessments. The actual timing of the samples may have been modified and/or up to an additional 2 samples may have been requested by the SRC after review of the data from previous cohorts. It was important that PK sampling occurred as closely as possible to the scheduled time (+10%). Both blood and urine samples were used for PK assessments.

In addition, for subjects who received placebo, a single plasma sample near the predicted tₘₐₓ of Compound I was evaluated to confirm the lack of circulating Compound I. Plasma concentration data for Compound I was summarized using descriptive statistics, including mean, standard deviation (SD), median, minimum, and maximum values, and percent coefficient of variation. Other PK parameters included (but were not limited to) Cₘₐₓ, tₘₐₓ, AUC, t_{1/2}, and MRT. Additionally, the apparent terminal-phase terminal half-life was calculated. The dose proportionality of AUC and Cₘₐₓ was explored.

### Study Results

### Plasma Concentrations of Compound I

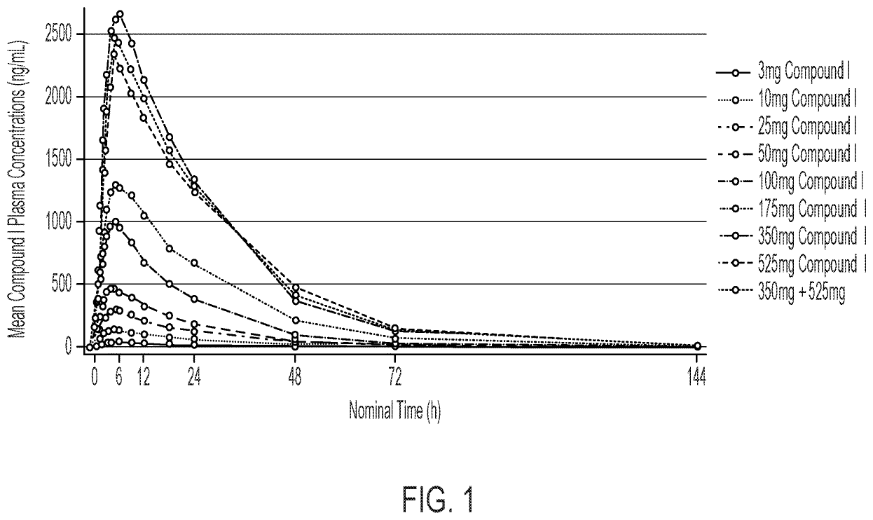

Plasma Compound I concentrations over time are summarized in Table 4 and FIG. 1.

TABLE 4
Summary of Compound I Plasma Concentrations by Treatment Group*

| | | | | | | | | | |
|---|---|---|---|---|---|---|---|---|---|
| | | | | | | | | | 350 mg + |
| Time Point | 3 mg | 10 mg | 25 mg | 50 mg | 100 mg | 175 mg | 350 mg | 525 mg | 525 mg |
| Statistic | (N = 6) | (N = 6) | (N = 6) | (N = 6) | (N = 6) | (N = 6) | (N = 6) | (N = 6) | (N = 12) |

6 Hours Postdose, ng/mL

| | | | | | | | | | |
|---|---|---|---|---|---|---|---|---|---|
| n | 6 | 6 | 6 | 6 | 6 | 6 | 6 | 6 | 12 |
| Mean | 35.833 | 130.933 | 286.333 | 431.333 | 947.167 | 1270.833 | 2660.000 | 2215.000 | 2437.500 |
| (SD) | (7.868) | (21.257) | (58.181) | (91.248) | (169.840) | (214.765) | (515.364) | (543.203) | (555.749) |
| Median | 33.500 | 137.000 | 286.500 | 420.500 | 935.000 | 1250.000 | 2550.000 | 2215.000 | 2350.000 |
| (Min, Max) | (28.10, | (91.60, | (190.00, | (344.00, | (683.00, | (955.00, | (2050.00, | (1490.00, | (1490.00, |
| | 49.50) | 154.00) | 354.00) | 603.00) | 1180.00) | 1520.00) | 3310.00) | 3070.00) | 3310.00) |
| Geometric | 35.173 | 129.288 | 280.892 | 424.188 | 933.866 | 1255.232 | 2618.926 | 2159.422 | 2378.102 |
| Mean | (20.978) | (18.207) | (22.370) | (19.691) | (18.875) | (17.528) | (19.482) | (25.247) | (23.816) |
| (CV % of GM) | | | | | | | | | |

12 Hours Postdose, ng/mL

| | | | | | | | | | |
|---|---|---|---|---|---|---|---|---|---|
| n | 6 | 6 | 6 | 6 | 6 | 6 | 6 | 6 | 12 |
| Mean | 21.883 | 94.217 | 211.167 | 321.500 | 674.500 | 1054.167 | 2136.667 | 1838.333 | 1987.500 |
| (SD) | (4.204) | (16.306) | (51.309) | (61.027) | (148.850) | (208.803) | (462.673) | (491.586) | (481.062) |
| Median | 21.700 | 97.750 | 223.000 | 297.000 | 637.000 | 1038.000 | 2115.000 | 1785.000 | 1865.000 |
| (Min, Max) | (16.30, | (63.70, | (131.00, | (278.00, | (497.00, | (744.00, | (1630.00, | (1190.00, | (1190.00, |
| | 27.10) | 109.00) | 272.00) | 443.00) | 905.00) | 1290.00) | 2660.00) | 2540.00) | 2660.00) |
| Geometric | 21.540 | 92.843 | 205.343 | 317.375 | 661.307 | 1036.021 | 2094.579 | 1782.754 | 1932.387 |
| Mean | (19.826) | (19.759) | (27.278) | (17.068) | (21.907) | (20.970) | (22.194) | (27.930) | (25.518) |
| (CV % of GM) | | | | | | | | | |

24 Hours Postdose, ng/mL

| | | | | | | | | | |
|---|---|---|---|---|---|---|---|---|---|
| n | 6 | 6 | 6 | 6 | 6 | 6 | 6 | 6 | 12 |
| Mean | 11.340 | 57.800 | 118.417 | 177.833 | 383.500 | 663.833 | 1321.167 | 1234.167 | 1277.667 |
| (SD) | (2.999) | (13.142) | (33.351) | (33.379) | (93.716) | (146.981) | (372.483) | (370.618) | (357.162) |
| Median | 10.795 | 61.450 | 124.000 | 174.500 | 372.000 | 689.500 | 1340.000 | 1180.000 | 1210.000 |
| (Min, Max) | (8.10, | (33.70, | (65.20, | (141.00, | (288.00, | (476.00, | (898.00, | (842.00, | (842.00, |
| | 15.20) | 69.20) | 158.00) | 232.00) | 498.00) | 816.00) | 1710.00) | 1690.00) | 1710.00) |
| Geometric | 11.014 | 56.271 | 113.883 | 175.322 | 373.957 | 649.568 | 1275.869 | 1188.060 | 1231.182 |
| Mean | (26.909) | (27.309) | (32.838) | (18.501) | (25.013) | (23.489) | (29.908) | (31.019) | (29.249) |
| (CV % of GM) | | | | | | | | | |

48 Hours Postdose, ng/mL

| | | | | | | | | | |
|---|---|---|---|---|---|---|---|---|---|
| n | 6 | 6 | 6 | 6 | 5 | 6 | 6 | 6 | 12 |
| Mean | 2.703 | 16.450 | 35.833 | 51.900 | 91.080 | 212.167 | 373.167 | 464.833 | 419.000 |
| (SD) | (1.618) | (5.022) | (13.945) | (15.835) | (48.402) | (65.184) | (193.509) | (231.575) | (209.018) |
| Median | 2.340 | 15.400 | 35.500 | 57.350 | 104.000 | 224.000 | 350.000 | 426.500 | 415.000 |
| (Min, Max) | (1.20, | (10.10, | (16.20, | (26.90, | (12.80, | (132.00, | (169.00, | (240.00, | (169.00, |
| | 4.81) | 24.90) | 53.60) | 68.10) | 145.00) | 279.00) | 616.00) | 853.00) | 853.00) |
| Geometric | 2.293 | 15.837 | 33.284 | 49.489 | 70.956 | 203.134 | 329.254 | 420.074 | 371.902 |
| Mean | (70.993) | (30.898) | (46.210) | (36.761) | (125.683) | (34.024) | (61.074) | (52.436) | (55.755) |
| (CV % of GM) | | | | | | | | | |

72 Hours Postdose, ng/mL

| | | | | | | | | | |
|---|---|---|---|---|---|---|---|---|---|
| n | 6 | 6 | 6 | 6 | 5 | 6 | 6 | 6 | 12 |
| Mean | 0.586 | 4.927 | 9.782 | 15.197 | 23.220 | 71.317 | 127.833 | 146.950 | 137.392 |
| (SD) | (0.666) | (2.573) | (5.557) | (6.579) | (15.892) | (33.581) | (89.934) | (112.650) | (97.695) |
| Median | 0.449 | 4.015 | 8.510 | 18.750 | 24.200 | 73.150 | 111.450 | 121.500 | 121.500 |
| (Min, Max) | (0.00, | (2.85, | (2.96, | (4.77, | (0.00, | (33.20, | (40.10, | (46.30, | (40.10, |
| | 1.46) | 9.89) | 18.80) | 20.00) | 44.80) | 111.00) | 264.00) | 350.00) | 350.00) |
| Geometric | CND | 4.503 | 8.401 | 13.495 | CND | 64.001 | 100.706 | 115.721 | 107.953 |
| Mean | | (46.107) | (70.609) | (64.870) | | (56.846) | (92.323) | (88.854) | (85.630) |
| (CV % of GM) | | | | | | | | | |

*The LLOQ is 0.5. Concentrations below the LLOQ are set to zero (0).
Abbreviations: CV % = percent of coefficient of variation; GM = geometric mean; CND = could not be determined; LLOQ = lower limit of quantification; Max = maximum; Min = minimum; n = number of subjects with assessment at the timepoint being summarized; N = number of subjects in the PK Population for the specified treatment; SD = standard deviation.

The results show that eight cohorts (48 subjects) were dosed safely up to 525 mg single dose. Compound I was detectable 48 hours postdose in all subjects and, in select doses and subjects, at 72 hours and 7 days postdose. At 7 days, Compound I was detectable in 24 subjects. Plasma samples for subjects on placebo were analyzed for all the time points; none of the 16 placebo subjects' plasma samples had any detectable Compound I levels.

The 525 mg group had slightly lower mean plasma concentrations relative to the 350 mg group up to the 24-hour time point; however, the 525 mg group had the highest plasma concentrations at the 48- and 72-hour time points. On Day 7, there was no Compound I detectable in plasma from the 3 mg Compound I group, while the drug was still detectable in all other groups. On Day 7, mean (SD) plasma concentrations (ng/mL) of Compound I were extremely low compared to the Cₘₐₓ and consistent with the expected concentrations based on the terminal t_{1/2} of about 15 hours.

### Plasma Pharmacokinetic Parameters of Compound I

Plasma PK parameters for Compound I are summarized in Table 5. Following oral administration of single-ascending doses of Compound I suspension, the peak plasma concentration occurred at approximately 4.5 to 5 hours across 8 dosing groups. The Cₘₐₓ, AUC₀₋ₜ, and AUC_{0-∞} increased with increasing Compound I dose up to 350 mg. The mean (SD) Cₘₐₓ was 2820 (478) ng/ml for the 350 mg dose group. The exposure after oral administration of 525 mg dose was similar to the exposure after 350 mg.

TABLE 5
Summary of Compound I Pharmacokinetic Parameters by Treatment Group*

| | | | | | | | | |
|---|---|---|---|---|---|---|---|---|
| | | | | | | | | 350 mg + |
| 3 mg | 10 mg | 25 mg | 50 mg | 100 mg | 175 mg | 350 mg | 525 mg | 525 mg |
| N = 6 | N = 6 | N = 6 | N = 6 | N = 6 | N = 6 | N = 6 | N = 6 | N = 12 |

Cₘₐₓ, ng/mL

| | | | | | | | | | |
|---|---|---|---|---|---|---|---|---|---|
| n | 6 | 6 | 6 | 6 | 6 | 6 | 6 | 6 | 12 |
| Mean | 38.18 | 139.17 | 303.50 | 500.33 | 1020.17 | 1316.17 | 2820.00 | 2350.00 | 2585.00 |
| (SD) | (7.15) | (19.53) | (60.68) | (118.88) | (198.81) | (209.65) | (478.00) | (565.97) | (556.51) |
| Median | 36.45 | 146.50 | 313.00 | 470.50 | 983.00 | 1305.00 | 2895.00 | 2240.00 | 2495.00 |
| (Min, Max) | (29.6, | (101.0, | (192.0, | (379.0, | (741.0, | (977.0, | (2210.0, | (1600.0, | (1600.0, |
| | 49.5) | 154.0) | 357.0) | 713.0) | 1340.0) | 1550.0) | 3310.0) | 3280.0) | 3310.0) |
| Geometric | 37.65 | 137.84 | 297.50 | 489.69 | 1004.13 | 1301.37 | 2785.31 | 2294.46 | 2528.00 |
| Mean | (18.47) | (15.82) | (23.25) | (22.51) | (19.75) | (16.89) | (17.51) | (24.33) | (22.67) |
| (% CV of | | | | | | | | | |
| GM) | | | | | | | | | |

Tₘₐₓ, hr

| | | | | | | | | | |
|---|---|---|---|---|---|---|---|---|---|
| n | 6 | 6 | 6 | 6 | 6 | 6 | 6 | 6 | 12 |
| Mean | 4.46 | 4.85 | 4.67 | 4.69 | 4.50 | 5.11 | 4.95 | 4.97 | 4.96 |
| (SD) | (1.21) | (0.74) | (0.52) | (0.49) | (0.84) | (0.68) | (1.04) | (0.58) | (0.80) |
| Median | 4.00 | 5.00 | 5.00 | 5.00 | 5.00 | 5.00 | 5.00 | 5.00 | 5.00 |
| (Min, Max) | (2.9, | (4.0, | (4.0, | (4.0, | (3.0, | (4.0, | (3.0, | (4.0, | (3.0, |
| | 5.9) | 5.9) | 5.0) | 5.0) | 5.0) | 5.8) | 5.9) | 5.8) | 5.9) |
| Geometric | 4.33 | 4.80 | 4.64 | 4.66 | 4.42 | 5.07 | 4.84 | 4.94 | 4.89 |
| Mean | (27.74) | (15.50) | (11.61) | (10.83) | (21.26) | (13.91) | (25.03) | (12.09) | (18.69) |
| (% CV of | | | | | | | | | |
| GM) | | | | | | | | | |

AUC₀₋₂₄, hr × ng/mL

| | | | | | | | | | |
|---|---|---|---|---|---|---|---|---|---|
| n | 6 | 6 | 6 | 6 | 6 | 6 | 6 | 6 | 12 |
| Mean | 521.20 | 2144.75 | 4652.27 | 7298.14 | 15372.00 | 22285.47 | 45569.07 | 38546.45 | 42057.76 |
| (SD) | (96.91) | (344.66) | (1008.21) | (1332.78) | (2844.48) | (3779.21) | (9694.46) | (9693.88) | (9944.03) |
| Median | 503.66 | 2249.99 | 4846.62 | 6733.64 | 14930.60 | 21729.75 | 44957.59 | 38212.84 | 38537.03 |
| (Min, Max) | (395.6, | (1487.1, | (2954.9, | (6460.5, | (11637.9, | (16630.7, | (35723.9, | (25167.0, | (25167.0, |
| | 664.1) | 2400.6) | 5755.3) | 9979.1) | 19842.9) | 26624.3) | 55941.6) | 52774.7) | 55941.6) |
| Geometric | 513.76 | 2117.77 | 4548.59 | 7211.76 | 15156.27 | 22008.68 | 44703.88 | 37503.94 | 40945.96 |
| Mean | (18.74) | (18.31) | (24.49) | (16.36) | (18.56) | (17.66) | (21.76) | (26.42) | (24.89) |
| (% CV of | | | | | | | | | |
| GM) | | | | | | | | | |

AUC₀₋₄₈, hr × ng/mL

| | | | | | | | | | |
|---|---|---|---|---|---|---|---|---|---|
| n | 6 | 6 | 6 | 6 | 6 | 6 | 6 | 6 | 12 |
| Mean | 689.10 | 3033.34 | 6497.67 | 10053.15 | 21379.14 | 32774.60 | 65860.61 | 58877.90 | 62369.25 |
| (SD) | (145.44) | (546.02) | (1554.87) | (1828.10) | (3963.01) | (6133.36) | (16205.91) | (16278.24) | (15909.80) |
| Median | 678.00 | 3190.31 | 6890.85 | 9502.16 | 21561.98 | 32704.35 | 66430.88 | 57440.02 | 57440.02 |
| (Min, Max) | (507.5, | (2010.9, | (3927.8, | (8620.0, | (16159.3, | (24021.9, | (49074.1, | (38106.2, | (38106.2, |
| | 896.9) | 3455.0) | 8217.7) | 13588.6) | 27033.7) | 39522.5) | 83761.3) | 80113.2) | 83761.3) |
| Geometric | 676.32 | 2985.00 | 6319.53 | 9931.55 | 21069.36 | 32277.46 | 64173.27 | 56964.59 | 60461.59 |
| Mean | (21.49) | (20.74) | (27.38) | (16.68) | (18.99) | (19.57) | (25.49) | (29.04) | (26.79) |
| (% CV of | | | | | | | | | |
| GM) | | | | | | | | | |

AUC_{0-∞}, hr × ng/mL

| | | | | | | | | | |
|---|---|---|---|---|---|---|---|---|---|
| n | 6 | 6 | 6 | 6 | 5 | 6 | 6 | 6 | 12 |
| Mean | 740.70 | 3412.28 | 7289.3 | 11329.27 | 21664.53 | 39069.40 | 76661.62 | 72056.15 | 74358.88 |
| (SD) | 180.64) | (685.53) | (1925.82) | (2104.19) | (4378.53) | (8328.43) | (23429.69) | (24586.79) | (23023.58) |
| Median | 729.66 | 3481.60 | 7570.55 | 11204.30 | 18749.02 | 39707.47 | 76241.22 | 68768.28 | 68768.28 |
| (Min, Max) | (524.8, | (2220.4, | (4204.3, | (9277.7, | (18177.4, | (28087.4, | (52341.8, | (42343.3, | (42343.3, |
| | 983.4) | 4212.0) | 9204.9) | 15028.5) | 26571.5) | 48554.7) | 105205.5) | 104410.0) | 105205.5) |
| Geometric | 722.23 | 3346.95 | 7045.70 | 11177.07 | 21324.23 | 38301.17 | 73626.42 | 68514.68 | 71024.58 |
| Mean | (25.11) | (22.63) | (30.43) | (17.91) | (19.91) | (22.36) | (32.16) | (36.28) | (32.83) |
| (% CV of | | | | | | | | | |
| GM) | | | | | | | | | |

AUC₀₋ₗₐₛₜ, hr × ng/mL

| | | | | | | | | | |
|---|---|---|---|---|---|---|---|---|---|
| n | 6 | 6 | 6 | 6 | 6 | 6 | 6 | 6 | 12 |
| Mean | 720.56 | 3350.32 | 7161.62 | 11218.73 | 22666.33 | 38996.88 | 76267.18 | 71666.80 | 73966.99 |
| (SD) | (177.81) | (691.45) | (1887.97) | (2015.76) | (7061.08) | (8283.03) | (23507.92) | (24469.14) | (23002.56) |
| Median | 713.41 | 3418.54 | 7416.23 | 11185.34 | 22401.00 | 39620.79 | 76161.9 | 68639.33 | 68639.33 |
| (Min, Max) | (507.5, | (2165.4, | (4146.4, | (9201.4, | (13770.3, | (28058.7, | (51630.1, | (41541.8, | (41541.8, |
| | 961.7) | 4198.2) | 9019.1) | 14645.6) | 32820.2) | 48418.5) | 104627.9) | 103276.5) | 104627.9) |
| Geometric | 702.17 | 3283.09 | 6924.51 | 11075.03 | 21734.19 | 38235.62 | 73185.05 | 68111.84 | 70602.89 |
| Mean | (25.42) | (23.15) | (30.23) | (17.56) | (32.94) | (22.28) | (32.55) | (36.55) | (33.13) |
| (% CV of | | | | | | | | | |
| GM) | | | | | | | | | |

Extrapolation, %

| | | | | | | | | | |
|---|---|---|---|---|---|---|---|---|---|
| n | 6 | 6 | 6 | 6 | 5 | 6 | 6 | 6 | 12 |
| Mean | 2.78 | 1.90 | 1.72 | 0.91 | 5.48 | 0.17 | 0.60 | 0.59 | 0.59 |
| (SD) | (0.64) | (0.86) | (0.85) | (1.01) | (11.78) | (0.10) | (0.55) | (0.75) | (0.63) |
| Median | 2.82 | 2.24 | 1.92 | 0.46 | 0.14 | 0.16 | 0.43 | 0.23 | 0.32 |
| (Min, Max) | (2.0, | (0.3, | (0.4, | (0.1, | (0.0, | (0.0, | (0.1, | (0.0, | (0.0, |
| | 3.5) | 2.6) | 2.8) | 2.5) | 26.6) | 0.3) | 1.4) | 1.9) | 1.9) |
| Geometric | 2.71 | 1.59 | 1.45 | 0.46 | 0.36 | 0.14 | 0.37 | 0.22 | 0.29 |
| Mean | (24.27) | (94.55) | (84.00) | (223.16) | (3059.38) | (80.84) | (166.79) | (388.75) | (244.12) |
| (% CV of | | | | | | | | | |
| GM) | | | | | | | | | |

V_{z}/F, L

| | | | | | | | | | |
|---|---|---|---|---|---|---|---|---|---|
| n | 6 | 6 | 6 | 6 | 5 | 6 | 6 | 6 | 12 |
| Mean | 66.91 | 58.45 | 68.63 | 87.88 | 77.91 | 106.02 | 98.81 | 165.90 | 132.36 |
| (SD) | (7.71) | (14.89) | (15.55) | (15.99) | (22.83) | (25.84) | (11.41) | (36.37) | (43.45) |
| Median | 65.4 | 55.83 | 66.45 | 90.60 | 82.08 | 100.58 | 98.36 | 171.02 | 116.02 |
| (Min, Max) | (56.9, | (44.4, | (52.6, | (64.3, | (45.3, | (85.8, | (84.1, | (118.0, | (84.1, |
| | 78.5) | 86.8) | 96.2) | 108.2) | 107.0) | 156.4) | 114.0) | 214.6) | 214.6) |
| Geometric | 66.54 | 57.09 | 67.29 | 86.60 | 74.94 | 103.80 | 98.26 | 162.47 | 126.35 |
| Mean | (11.47) | (23.35) | (21.67) | (19.19) | (33.12) | (21.90) | (11.63) | (22.94) | (32.16) |
| (% CV of | | | | | | | | | |
| GM) | | | | | | | | | |

CL/F, L/hr

| | | | | | | | | | |
|---|---|---|---|---|---|---|---|---|---|
| n | 6 | 6 | 6 | 6 | 5 | 6 | 6 | 6 | 12 |
| Mean | 4.26 | 3.06 | 3.69 | 4.53 | 4.76 | 4.66 | 4.95 | 8.07 | 6.51 |
| (SD) | (1.05) | (0.76) | (1.22) | (0.77) | (0.90) | (1.05) | (1.53) | (2.84) | (2.72) |
| Median | 4.22 | 2.88 | 3.32 | 4.46 | 5.33 | 4.45 | 4.75 | 7.64 | 6.10 |
| (Min, Max) | (3.1, | (2.4, | (2.7, | (3.3, | (3.8, | (3.6, | (3.3, | (5.0, | (3.3, |
| | 5.7) | 4.5) | 5.9) | 5.4) | 5.5) | 6.2) | 6.7) | 12.4) | 12.4) |
| Geometric | 4.15 | 2.99 | 3.55 | 4.47 | 4.69 | 4.57 | 4.75 | 7.66 | 6.04 |
| Mean | (25.11) | (22.63) | (30.43) | (17.91) | (19.91) | (22.36) | (32.16) | (36.28) | (42.09) |
| (% CV of | | | | | | | | | |
| GM) | | | | | | | | | |

Half-Life Lambda Z, hr

| | | | | | | | | | |
|---|---|---|---|---|---|---|---|---|---|
| n | 6 | 6 | 6 | 6 | 5 | 6 | 6 | 6 | 12 |
| Mean | 11.32 | 13.36 | 13.28 | 13.61 | 11.66 | 15.83 | 14.66 | 15.07 | 14.87 |
| (SD) | (2.41) | (2.02) | (2.24) | (2.43) | (3.62) | (1.72) | (3.41) | (3.88) | (3.49) |
| Median | 11.18 | 12.86 | 12.68 | 13.81 | 12.53 | 16.36 | 14.54 | 13.77 | 14.54 |
| (Min, Max) | (8.8, | (11.3, | (11.2, | (9.7, | (5.7, | (12.9, | (10.8, | (11.9, | (10.8, |
| | 14.5) | 17.2) | 17.6) | 16.6) | 15.1) | 17.4) | 19.6) | 21.7) | 21.7) |
| Geometric | 11.10 | 13.24 | 13.14 | 13.42 | 11.08 | 15.75 | 14.33 | 14.70 | 14.51 |
| Mean | (21.54) | (14.20) | (15.62) | (19.18) | (40.10) | (11.49) | (23.73) | (24.53) | (23.02) |
| (% CV of | | | | | | | | | |
| GM) | | | | | | | | | |

Mean Residence Time, hr

| | | | | | | | | | |
|---|---|---|---|---|---|---|---|---|---|
| n | 6 | 6 | 6 | 6 | 5 | 6 | 6 | 6 | 12 |
| Mean | 17.48 | 21.20 | 20.75 | 20.98 | 19.48 | 24.56 | 22.77 | 25.78 | 24.28 |
| (SD) | (3.13) | (2.76) | (2.84) | (2.99) | (3.96) | (2.66) | (4.15) | (4.77) | (4.54) |
| Median | 16.80 | 20.11 | 20.07 | 21.47 | 20.35 | 24.43 | 22.73 | 24.96 | 23.48 |
| (Min, Max) | (14.3, | (19.4, | (17.7, | (16.4, | (13.5, | (20.2, | (17.9, | (21.4, | (17.9, |
| | 22.5) | 26.6) | 25.9) | 23.9) | 23.9) | 27.9) | 28.3) | 34.5) | 34.5) |
| Geometric | 17.26 | 21.06 | 20.60 | 20.79 | 19.13 | 24.44 | 22.45 | 25.45 | 23.90 |
| Mean | (17.56) | (12.06) | (13.10) | (14.93) | (22.32) | (11.19) | (18.57) | (17.45) | (18.41) |
| (% CV of | | | | | | | | | |
| GM) | | | | | | | | | |

*Abbreviations: AUC₀₋₂₄ = area under plasma concentration-time curve from 0 to 24 hours; AUC₀₋₄₈ = area under plasma concentration-time curve from 0 to 48 hours; AUC_{0-∞} = area under plasma concentration-time curve from 0 to infinity; AUC₀₋ₗₐₛₜ = area under plasma concentration-time curve from 0 to the last measurable concentration; CL/F = apparent total clearance of drug from plasma after oral administration uncorrected for bioavailability; Cₘₐₓ = maximum observed plasma concentration; CND = could not be determined; % CV = percent of coefficient of variation; GM = geometric mean; Max = maximum; Min = minimum; N = number of subjects in the PK Population for the specified treatment; n = number of subjects with assessments for the parameter being summarized; PK = pharmacokinetic; tₘₐₓ = time of maximum observed plasma concentration; Vz/F = terminal volume of distribution uncorrected for bioavailability. Concentrations below the lower limit of quantification were set to zero (0).

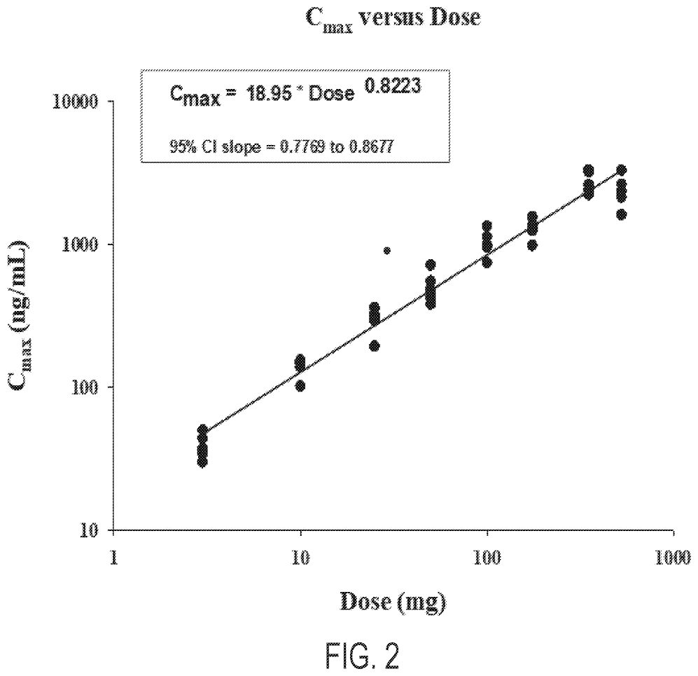

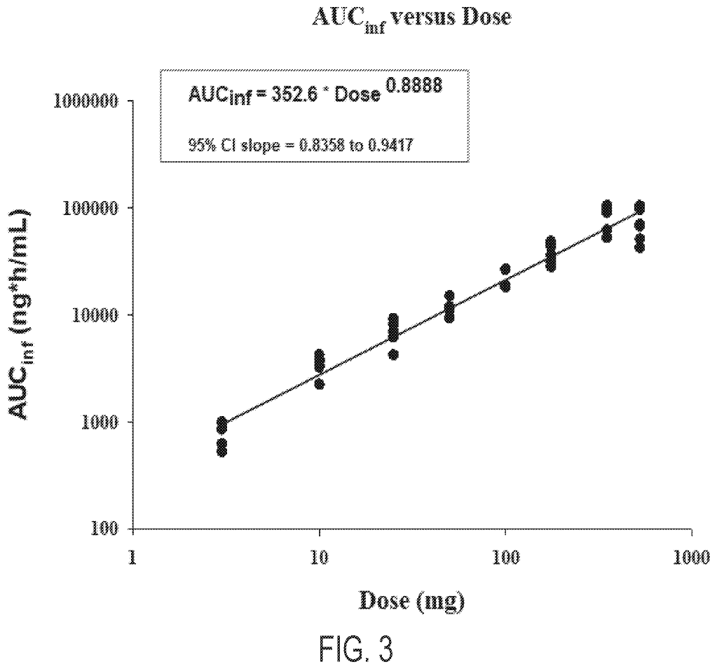

Dose proportionality was assessed using a power model. The plots for Cₘₐₓ and AUC_{inf} versus dose are displayed in FIGS. 2 and 3, respectively. There appeared to be an approximately direct relationship but slightly less than dose-proportional (slope=0.8888, 95% CI Interval=0.8358-0.9417) with doses through the 350 mg dose group, and a less than dose-proportional response at the 525 mg dose. Thus, an additional sensitivity analysis was performed to assess dose-proportionality with AUC data from the 525 mg group excluded; this analysis found that the dose response was nearly dose-proportional up to 350 mg Compound I as the slope was less than 1.0 (slope=0.9347; 95% CI Interval=0.8813-0.9882).

The elimination of Compound I appeared to be monoexponential (FIG. 1). The terminal t_{1/2} was approximately 11-16 hours across the dose groups (Table 5). The apparent oral clearance (CL/F) and volume of distribution (Vz/F) were estimated to be approximately 3.1 to 8.1 L/h and 58 to 166 L, respectively, for the doses ranging from 3 mg to 525 mg. Both CL/F and Vz/F at higher doses increased with increasing dose, suggesting that fraction of absorption was reduced at highest doses; this could result from, for example, limited solubility, slow dissolution, and/or fecal excretion of undissolved Compound I molecules. It has now been determined that Compound I is a Biopharmaceutics Classification System (BCS) Class II compound. The decreased exposure in the 525 mg cohort likely resulted from slow dissolution due to poor solubility of Compound I and incomplete absorption of undissolved drug molecules in the gastrointestinal tract. The mean apparent clearance and volume of distribution were approximately 4.2 L/h and 78 L, respectively, for doses up to 175 mg.

The cumulative urinary excretion of unchanged Compound I over the 48-hours postdose collection period (Ae₀₋₄₈ₕ) increased with the increasing doses from 3 to 525 mg. Approximately 12% (range of 3.9%-23.9%) of the Compound I dose was recovered in 0-48 hours urine collection as unchanged Compound I after oral administration of 3 to 175 mg doses. At doses of 350 and 525 mg, the percentages of doses recovered in 0-48 hours urine collection were approximately 6.0% and 8.6%, respectively. The decreased urinary excretion of Compound I in 0-48 hours urine at high doses was likely caused by (1) lower fraction of absorption at high doses due to limited solubility; and (2) incomplete urinary excretion within 48 hours postdose.

Renal clearance appeared to be independent of dose with a mean value of approximately 0.570 L/h (or 9.5 mL/min) (individuals ranged from 0.177 to 1.400 L/h). The intersubject variability in renal clearance (CLᵣ) was moderate with the percent coefficient of variation (% CV) ranging from 32% to 80% in 8 cohorts. The renal clearance was lowest in the 350 mg dose group with a mean (SD) value of 0.333 (0.135) L/h and highest in the 525 mg dose group with a mean (SD) value of 0.800 (0.319) L/h. The variability in CLᵣ was relatively larger than total plasma clearance (CL/F). Renal clearance may be influenced by multiple factors including physiology parameters, e.g., renal blood flow, urine flow, renal function, urine volume, and urine pH. Renal excretion of Compound I and renal clearance would be affected as these parameters vary in individuals.

Renal clearance depends on glomerular filtration rate, tubular active secretion, and tubular reabsorption. The extent to which a drug is filtered depends on the molecular size, protein binding, ionization, polarity, and kidney function. If CLᵣ depends only on filtration, then CLᵣ=GFR*fᵤ, where fᵤ is the unbound fraction of drug and GFR is the glomerular filtration rate. Renal clearance observed in this study was close to GFR*fᵤ (e.g., GFR=100 mL/min for subjects with normal renal function and fᵤ-14 to 18% for the free fraction of Compound I in plasma), suggesting that glomerular filtration is the major mechanism for the renal elimination of Compound I.

### PK Conclusions

The above data show that Compound I exposure (Cₘₐₓ and AUC_{0-∞}) increased in an almost linear, close to dose-proportional manner through the 350 mg dose. At the 525 mg dose, no further increase in exposure was observed vs. the 350 mg dose; this was likely due to decreased fraction of absorption (lower oral bioavailability). As the exposure of the 350 and 525 mg cohorts were similar, the data from the 2 groups were combined and resulted in a mean Cₘₐₓ of 2585 ng/ml and AUC_{0-∞} of 74359 ng×h/mL, mean tₘₐₓ of 5 hours, and mean terminal t_{1/2} of approximately 15 hours. The range in the combined 350 and 525 mg group was 3 to 6 hours for tₘₐₓ and 11 to 22 hours for t_{1/2}. The data also show that Tₘₐₓ and t_{1/2} were dose-independent. At doses up to 175 mg, the apparent total oral clearance (CL/F) averaged 4.2 L/h, suggesting that Compound I is a low clearance drug, and the apparent volume of distribution (Vz/F) 78 L, indicating extensive tissue distribution. Both values were higher in the 525 mg dose group, supporting the hypothesis of decreased oral bioavailability at doses >350 mg. The data also show that approximately 12% of the administered dose was excreted in urine as unchanged Compound I at doses <350 mg. This value was lower for the two highest dose groups which is likely due to incomplete recovery of all drug excreted in the 48 hours urine collection and possibly decreased oral bioavailability at the highest doses. Renal clearance was largely dose-independent (mean 0.57 L/h). The renal clearance of Compound I was close to the product of glomerular filtration rate by unbound fraction of Compound I in plasma, implying that glomerular filtration is likely the major mechanism of renal excretion.

### Analysis of Pharmacodynamics

The expected pharmacological effect of Compound I would result in an increase in contractility that would translate into an increase in LVFS, LVEF, LVSV, LVOT-VTI and a possible decrease in left ventricular end-systolic diameter (LVESD) and left ventricular end-systolic volume (LVESV). Echocardiographic parameters demonstrated the expected intra- and inter-subject variability as reflected in the serial measurements obtained in the placebo group; thus, changes in the TTE measurements that were in the opposite direction than consistent with the pharmacology of Compound I likely were mostly a reflection of the intra- and inter-subject variation in the TTE measurements. Some of the variation was also reflected in the recording in the subjects who received placebo.

### Systolic Ejection Time

SET was determined as a safety parameter, as administration of the myosin modulator omecamtiv to healthy volunteers at high doses resulted in ischemia that appeared to correlate with a significant increase in the SET. With Compound I, after administration of the higher dose levels (175 mg through 525 mg) there was an increase of SET that peaked at about 1.5 to 2 hours. This was before the maximum plasma concentration of Compound I was observed. The largest observed mean (SD) increase in SET was recorded for the 350 mg Compound I group at 19.2 (20.5) msec 1.5 to 2 hours postdose. The observed mean (SD) increase in SET for the 350 mg and 525 mg Compound I combined dose group was 18.0 (19.5) msec at 1.5 to 2 hours postdose. In all except the 3 mg and 10 mg groups, the mean SET change from Baseline peaked at approximately 1.5-2 hours postdose. SET trended upward at the last measurement (24 hours postdose) when the plasma concentrations were significantly lower than at Cₘₐₓ. A transient decrease in the SET was observed, mostly on placebo and at the lower doses, this decrease probably being a reflection of diurnal variability of the measurement.

### Left Ventricular Outflow Tract-Velocity Time Integral

Resting LVOT-VTI showed a peak mean absolute change from Baseline at approximately 6 and 12 hours postdose. The maximum LVOT-VTI observed was 2.54 (1.78) cm 6 hours postdose in the 350 mg group. The observed mean (SD) increase in LVOT-VTI for the 350 mg and 525 mg Compound I combined dose group was 2.28 (1.43) cm at 6 hours postdose. The majority of the values remained at or below Baseline after 24 hours postdose.

### Left Ventricular Ejection Fraction

Mean resting LVEFs were measured. There were time-dependent changes in resting LVEF, with the earlier peak increase occurring about 6 hours after dosing consistent with the approximate tₘₐₓ. The values returned to approximately Baseline by the 24 hours TTE. The maximum mean (SD) increases were 4.65 (1.45) % observed 6 hours postdose in the 525 mg Compound I group and 4.83 (2.65) % observed 12 hours postdose in the 100 mg Compound I group.

### Left Ventricular Stroke Volume

Mean resting LVSVs were measured. At 6 and 12 hours postdose, all dose groups demonstrated an increase in stroke volume as compared to the measurement at Baseline. The maximum mean (SD) increase was 10.848 (9.893) mL observed 12 hours postdose in the 350 mg Compound I group. The mean (SD) increase in LVSV for the 350 mg and 525 mg Compound I combined dose group was 7.623 (7.842) mL at 12 hours postdose. The majority of groups were at or below Baseline at 24 hours postdose. The 350 mg group mean was trending toward Baseline at 24 hours postdose.

### Left Ventricular Fractional Shortening

An increase in LVFS was observed in the higher dose cohorts, with the maximum increase occurring at the 6 hours TTE, which was about the time of the maximum plasma concentration. At the lower doses, there was little change in the LVFS over time, with the change from Baseline within the variation of the measurement.

### Left Ventricular End-Systolic Diameter

Resting LVESD decreased in an approximately dose- and time-dependent manner, with the exception of the 3 mg Compound I group. The largest observed mean (SD) decrease was −0.455 (0.357) cm 12 hours postdose in the 525 mg group. The changes remained below Baseline through 24 hours postdose for the majority of the dose groups, although the changes were trending toward Baseline values.

### Left Ventricular End-Systolic Volume

Resting LVESV decreased overall in a generally dose-dependent manner. The minimum LVESV (at approximately 6 hours postdose) appeared to be dose-dependent, as the largest mean (SD) decrease observed was −9.21 (3.18) mL 6 hours postdose in the 525 mg group. The observed mean (SD) decrease in LVESV for the 350 mg and 525 mg Compound I combined dose group was −6.82 (5.99) mL at 6 hours postdose. The majority of the values remained below Baseline at 24 hours postdose.

### Left Ventricular End-Diastolic Diameter

Resting left ventricular end-diastolic diameter (LVEDD) did not have dose- or time-dependent trends, but at doses of 100 mg through 525 mg there was a slight decrease in LVEDD from 1.5-2 to 12 hours postdose. The largest observed mean (SD) decrease was-0.213 (0.221) cm 12 hours postdose in the 525 mg Compound I group. The mean (SD) decrease in LVEDD for the 350 mg and 525 mg Compound I combined dose group was-0.171 (0.177) cm at 12 hours postdose. The highest observed change from Baseline at 24 hours postdose was 0.103 (0.217) cm in the 50 mg group.

### Left Ventricular End-Diastolic Volume

Resting left ventricular end-diastolic volume (LVEDV) decreased overall in a generally dose-dependent trend. The decreases (at approximately 6 hours postdose) appeared to be dose-dependent, as the largest mean (SD) decrease observed was-12.5 (6.96) mL 6 hours postdose in the 525 mg group. The mean (SD) decrease in LVEDV for the 350 mg and 525 mg Compound I combined dose group was-9.98 (7.83) mL at 6 hours postdose. The majority of the values remained below Baseline after 24 hours postdose.

### Left Ventricular Pre-Ejection Period

Resting pre-ejection period (PEP) showed a peak mean absolute change from Baseline at approximately 1.5 to 2 and 8 to 9 hours postdose, with a minimum at approximately 6 hours postdose. The maximum LV pre-ejection period observed was trending positive (above Baseline) at 24 hours postdose in most dose groups.

### Isovolumic Contraction Time

Resting isovolumic contraction time (IVCT) showed a decrease in mean absolute change from Baseline at approximately 6 and 12 hours postdose. The maximum IVCT observed was trending positive (toward Baseline) at 24 hours postdose in most dose groups.

### Isovolumic Relaxation Time

Resting isovolumic relaxation time (IVRT) showed an increase in mean absolute change from Baseline at approximately 1.5 to 2 hours and 8 to 9 hours postdose. The mean IVRT was trending positive at 24 hours postdose.

### Drug Dose, Drug Concentration, and Relationship to Response

As the Cₘₐₓ occurred at between 4 and 6 hours in most of the subjects, the TTE obtained at 6 hours postdose was considered the best timepoint to explore the relationship between concentration and pharmacological effect. TTEs obtained at 1.5 and 3 hours after dosing were before the Cₘₐₓ and at 9 hours were after the peak Cₘₐₓ. Based on the preclinical data, it was considered unlikely that there would be a prolonged lag between the Cₘₐₓ and peak pharmacological effect. As the exposure after the administration of the 350 mg and 525 mg doses were very similar, it was decided to not only present the results from these groups separately, but also to combine the data from these groups. By combining the data from the 2 groups, the number of subjects dosed was increased from 6 to 12, thus increasing the power to observe a statistically significant change from Baseline in the TTE parameters of interest.

In the 525 mg dose group at 6 hours postdose, there were statistically significant differences (unadjusted p<0.001) in SET, LVESD, LVFS, and (unadjusted p<0.05) in IVCT at a mean (SD) plasma level of 2215 (543) ng/mL. For the 350 mg dose group at 6 hours postdose, there were statistically significant differences (unadjusted p<0.05) in SET, LVESD, LVFS, IVRT, and HR at the mean (SD) plasma level of 2660 (515) ng/mL. For the combined 350 mg and 525 mg dose groups at 6 hours postdose, statistically significant differences (unadjusted p<0.001) were observed in SET, LVESD, LVFS and (unadjusted p<0.05) in LVEF, IVRT at a mean (SD) plasma level of 2438 (556) ng/mL. Statistically significant differences were observed in several parameters at lower plasma Compound I plasma concentrations.

An analysis of placebo-corrected change from Baseline at 6 hours postdose by Compound I plasma concentration bins is presented in Table 6 below.

TABLE 6
Placebo-Corrected Change from Baseline in Selected TTE Parameters

| | | | Compound I Plasma Concentrations | | |
|---|---|---|---|---|---|
| | | | (ng/mL) (N = 48) | | |
| | | Baseline^{a} | 0-1000 | 1001-2000 | >2000 |
| Parameter | Statistic | (N = 48) | (n = 29) | (n = 9) | (n = 10) |

Plasma Concentrations

| | | | | | |
|---|---|---|---|---|---|
| Plasma | Mean | — | 334.3 | 1361.1 | 2592.0 |
| Concentration | SD | — | 295.2 | 244.5 | 459.9 |
| (ng/mL) | Median | — | 286 | 1270 | 2425 |
| | Q1, Q3 | — | 126, 424 | 1180, 1500 | 2280, 3070 |
| | Min, Max | — | 28, 955 | 1070, 1840 | 2050, 3310 |

Left Ventricular Outflow Doppler

| | | | | | |
|---|---|---|---|---|---|
| SET (ms) | Difference^{b} | 327.9 | 10.623 | 23.049 | 25.645 |
| | SE^{c} | 19.6 | 5.921 | 7.943 | 7.712 |
| | p-value^{d} | — | 0.0779 | 0.0052* | 0.0015* |
| Stroke Volume | Difference^{b} | 69.5 | 2.631 | 2.509 | 8.197 |
| (mL) | SE^{c} | 12.3 | 2.896 | 3.918 | 3.993 |
| | p-value^{d} | — | 0.3672 | 0.5244 | 0.0445* |

Two-Dimensional

| | | | | | |
|---|---|---|---|---|---|
| LVESD (cm) | Difference^{b} | 3.204 | 0.025 | −0.159 | −0.306 |
| | SE^{c} | 0.397 | 0.058 | 0.077 | 0.077 |
| | p-value^{d} | — | 0.6655 | 0.0431* | 0.0002** |
| LVEDD (cm) | Difference^{b} | 4.665 | −0.014 | −0.041 | −0.115 |
| | SE^{c} | 0.407 | 0.055 | 0.073 | 0.071 |
| | p-value^{d} | — | 0.8052 | 0.5781 | 0.1084 |
| LVFS (%) | Difference^{b} | 31.837 | −0.560 | 3.549 | 6.289 |
| | SE^{c} | 5.071 | 1.136 | 1.522 | 1.550 |
| | p-value^{d} | — | 0.6243 | 0.0233* | 0.0002** |
| LVESV (mL) | Difference^{b} | 33.981 | −1.204 | −4.101 | −6.031 |
| | SE^{c} | 8.679 | 1.572 | 1.945 | 1.873 |
| | p-value^{d} | — | 0.4471 | 0.0396* | 0.0021* |
| LVEDV (mL) | Difference^{b} | 92.671 | −2.210 | −6.100 | −9.678 |
| | SE^{c} | 19.633 | 2.516 | 3.067 | 2.945 |
| | p-value^{d} | — | 0.3835 | 0.0517 | 0.0018* |
| LVEF (%) | Difference^{b} | 63.450 | 0.322 | 2.176 | 3.219 |
| | SE^{c} | 4.188 | 1.169 | 1.525 | 1.479 |
| | p-value^{d} | — | 0.7836 | 0.1593 | 0.0338* |
| LVGLS (%) | Difference^{b} | −20.352 | −0.467 | −1.274 | −1.779 |
| | SE^{c} | 2.012 | 0.593 | 0.787 | 0.762 |
| | p-value^{d} | — | 0.4340 | 0.1108 | 0.0230* |
| LVGCS (%) | Difference^{b} | −29.829 | −1.076 | −1.383 | −2.854 |
| | SE^{c} | 2.491 | 0.749 | 0.963 | 0.988 |
| | p-value^{d} | — | 0.1561 | 0.1564 | 0.0055* |

Mitral Inflow Doppler

| | | | | | |
|---|---|---|---|---|---|
| Peak E (m/s) | Difference^{b} | 82.021 | −0.966 | −1.427 | −5.297 |
| | SE^{c} | 13.236 | 3.210 | 4.208 | 4.079 |
| | p-value^{d} | — | 0.7647 | 0.7358 | 0.1992 |
| Peak A (m/s) | Difference^{b} | 49.936 | −3.092 | −2.872 | 0.657 |
| | SE^{c} | 10.508 | 2.383 | 3.153 | 3.060 |
| | p-value^{d} | — | 0.1997 | 0.3662 | 0.8307 |
| E/A ratio | Difference^{b} | 1.947 | 0.150 | 0.177 | −0.047 |
| | SE^{c} | 0.518 | 0.105 | 0.136 | 0.132 |
| | p-value^{d} | — | 0.1609 | 0.1973 | 0.7214 |
| PEP (ms) | Difference^{b} | 86.750 | −2.190 | −5.487 | −2.131 |
| | SE^{c} | 11.405 | 2.862 | 3.915 | 3.689 |
| | p-value^{d} | — | 0.4472 | 0.1664 | 0.5657 |
| IVCT (ms) | Difference^{b} | 67.458 | 0.677 | 1.412 | 6.115 |
| | SE^{c} | 17.217 | 2.904 | 3.880 | 3.733 |
| | p-value^{d} | — | 0.8164 | 0.7172 | 0.1067 |
| IVRT (ms) | Difference^{b} | 73.750 | 1.526 | 0.148 | 11.986 |
| | SE^{c} | 12.152 | 2.806 | 3.744 | 3.915 |
| | p-value^{d} | — | 0.5886 | 0.9686 | 0.0033* |
| MPI | Difference^{b} | 0.433 | −0.007 | −0.021 | 0.033 |
| | SE^{c} | 0.082 | 0.016 | 0.021 | 0.021 |
| | p-value^{d} | — | 0.6639 | 0.3265 | 0.1188 |

Tissue Doppler

| | | | | | |
|---|---|---|---|---|---|
| E/e′(lateral) | Difference^{b} | 6.091 | −0.235 | 0.144 | −0.063 |
| | SE^{c} | 1.554 | 0.274 | 0.366 | 0.372 |
| | p-value^{d} | — | 0.3945 | 0.6959 | 0.8659 |

Vital Signs

| | | | | | |
|---|---|---|---|---|---|
| HR (bpm) | Difference^{b} | 58.40 | −1.070 | 0.336 | 2.871 |
| | SE^{c} | 8.50 | 2.192 | 2.818 | 2.746 |
| | p-value^{d} | — | 0.6272 | 0.9054 | 0.3002 |
| SBP (mm Hg) | Difference^{b} | 113.44 | −0.623 | 1.132 | −1.572 |
| | SE^{c} | 8.10 | 2.089 | 2.796 | 2.670 |
| | p-value^{d} | — | 0.7666 | 0.6871 | 0.5584 |
| DBP (mm Hg) | Difference^{b} | 66.04 | 2.277 | 2.937 | 1.540 |
| | SE^{c} | 6.79 | 1.674 | 2.219 | 2.150 |
| | p-value^{d} | — | 0.1790 | 0.1909 | 0.4766 |

Abbreviations: A = late peak wave velocity from mitral inflow Doppler; e′ = peak atrioventricular valve annular velocity in early diastole; E = early peak wave velocity from mitral inflow Doppler; bpm = beats per minute; IVCT = isovolumic contraction time; IVRT = isovolumic relaxation time; LS = least squares; LVEDD = left ventricular end-diastolic diameter; LVEF = left ventricular ejection fraction; LVESD = left ventricular end-systolic diameter; LVFS = left ventricular fractional shortening; LVGCS = left ventricular global circumferential strain; LVGLS = left ventricular global longitudinal strain; Max = maximum; Min = minimum, MPI = myocardial performance index; n = number of subjects in the group; N = number of subjects in the population; PEP = pre-ejection period; Q1 = quartile 1; Q3 = quartile 3; SD = standard deviation; SE = standard error; SET = systolic ejection time.
^{a}Absolute arithmetic mean values and SD for the Baseline measurement for all Compound I treated subjects, excluding the placebo subjects.
^{b}LS mean difference = placebo-corrected least square mean difference in LS means of change from Baseline to 6 hours post-dose values.
^{c}SE of LS mean difference = standard error of the least square mean difference.
^{d}p-values were computed using an analysis of covariance with effects for group and covariate of Baseline assessment, testing the null hypothesis whether the placebo-corrected change from Baseline in the concentration group was equal to zero or not.
*= statistically significant at the 0.05 level.
**= statistically significant at the 0.001 level.

As shown in Table 6, there were significant (unadjusted p<0.05) effects of Compound I in the 1001-2000 ng/mL range on SET, LVESD, LVFS, and LVESV. At Compound I plasma concentrations >2000 ng/ml (median 2425 ng/mL), there were significant effects (LS mean difference±SE) on SET (25.6±7.71 ms), stroke volume (8.20±3.99 mL), LVESD (−0.306±0.077 cm), LVFS (6.29±1.55%), L VESV (−6.03±1.87 mL), LVEDV (−9.68±2.95 mL), LVEF (3.22±1.48%), left ventricular global longitudinal strain (LVGLS) (−1.78=0.76 ms), left ventricular global circumferential strain (LVGCS) (−2.85±0.99 ms), and IVRT (as assessed by mitral inflow doppler) (12.0=3.92 ms). There were no significant effects on diastolic function/relaxation, based on no change in E/A ratio and E/e′; however, IVRT was significantly increased.

Additional analyses of the relationship between Compound I plasma concentrations and PD parameter responses were performed using Loess regression (Cleveland and Devlin, Journal of the American Statistical Association 84(403):596-610 (1988)). There were overall increases in SET, LVSV, LVOT-VTI, and LVFS associated with increasing Compound I plasma concentrations.

### PD Conclusions

The above PD data show that there was an apparent dose- and concentration-dependent, reversible increase in echocardiographic measures of forward flow and contractility with a concomitant decrease in LV volumes. The PD effects were discernable mostly at concentrations ≥1000 ng/ml; peak effect was observed at the TTE time point obtained closest to tₘₐₓ (6 hours) and had mostly returned to Baseline by 24 hours with the exception of the highest dose groups, where some effects on contractility remained. These changes were accompanied by only a modest increase in SET and limited adverse effect on diastolic function as evidenced by no consistent change in E/A and E/e′. For subjects whose concentration exceeded 2000 ng/ml (median concentration 2592 ng/mL) there were statistically significant changes from Baseline in the following parameters: a mean absolute increase in LVFS of 6.3%, a mean absolute increase in LVEF of 3.2%, a mean increase in LVSV of 8.2%, a mean increase in SET of 25.7 ms, a mean decrease in LVESD of 0.31 cm, a mean decrease in L VEDD of 0.12 cm, a mean decrease in LVESV of 6.03 mL, a mean decrease in LVEDV 9.68 mL, a mean absolute decrease in LVGLS of 1.78%, and a mean absolute decrease in LVGCS of 2.85%.

### Safety Evaluation

Fifty AEs were reported in 34 subjects overall. There was no trend for increasing AE frequency with Compound I dose and no apparent differences from pooled placebo, with the exception of cardiac arrhythmias which occurred more frequently in subjects receiving Compound I. All observed cardiac arrhythmias are known to occur spontaneously in healthy volunteers so this difference may be due to chance. All AEs were mild or moderate in severity. One subject had a serious AE of a short duration of complete AV block (100 mg Compound I dose group). At 16-22 hours postdosing, the subject had bradycardia (<50 beats per minute [bpm]) and 3 short episodes of complete heart block (4-8 sec each). The other possible AEs of concern, which were considered drug-related, included 3 subjects who received Compound I and had brief episodes of arrhythmia (1 subject with accelerated idioventricular rhythm, 1 subject with ventricular extrasystoles and 1 subject with an isolated nonsustained ventricular tachycardia (NSVT, 3 beats) observed on telemetry. It should be noted that such AEs can occur in healthy subjects. No subject discontinued due to an AE. AEs considered by the investigator to be related to treatment were reported in 3 subjects (50.0%) in the 350 mg and 50 mg Compound I dose groups and 1 subject in each of the remaining dose groups (except 25 mg Compound I, which had no related TEAEs reported).

In conclusion, the study shows that overall, Compound I was well-tolerated at doses up to 525 mg and no notable safety signals were identified during the study. Most AEs were mild or moderate in severity and most were unrelated to study drug. There was no trend for increased frequency or severity of AEs with increasing Compound I dose. The most common (occurring in ≥3 subjects) AEs were headache, fatigue, catheter site related reaction, back pain, dizziness, upper respiratory tract infection, and chest discomfort. Chest discomfort or noncardiac chest pain occurred in 4 subjects: 1 on placebo (2 hours postdose) and 3 on active drug (occurring 4 to 5 days after dosing with 10, 25, and 350 mg, respectively). The only AEs considered to be drug-related occurring in more than 1 subject were headache and chest discomfort. Episodes of headache were rated mild to moderate in severity. All episodes of chest discomfort were rated as mild. One of the 2 episodes of chest discomfort occurred after a 350 mg dose. The other episode of chest discomfort, and those of headache, occurred after lower doses of Compound I that were 50 mg or less.

One subject (001-136), a 31 year-old man receiving Compound I (100 mg) experienced 3 short (4 to 8 sec each) episodes of asymptomatic third degree AV heart block on telemetry during sleep 16 to 22 hours after dosing. The patient had no history of syncope or cardiac disease, although it should be noted that this subject had first-degree AV block and bradycardia on Screening and predose ECGs. This event was assessed by the investigator as mild in severity and possibly related to the study drug, whereas the Sponsor assessed the event as unrelated to the study drug (possible increased vagal tone during sleep).

Three other subjects receiving Compound I experienced arrhythmias 8.5 to 48 hours after the dose of Compound I. Each of the arrhythmias was the type that may be observed in healthy volunteers, of short duration (few seconds), and asymptomatic.

One subject experienced a mild increase in hs-troponin I (16 ng/L with upper range of normal being 15 ng/L). No troponin increase was observed in any other subject.

There were no significant changes on the ECG or ECG intervals including PR interval. The one instance of QTcF >450 msec was recorded in a subject that received a low dose (10 mg). No dose-dependent trends involving high QTcF were observed.

There were no clinically significant changes in vital sign or safety laboratory parameters.

### Troponin I

Troponin was measured using a high sensitivity human troponin assay (Abbott Architect STAT High Sensitivity Troponin I) with the upper limit of normal range being 15 ng/mL. Compound IA very slight increase in hs-troponin I concentration was seen in one subject (in the 525 mg Compound I treatment group), that of a value of 16 ng/ml at 6 hours postdose that was within the normal range 2 hours later. The subject had experienced PVCs at about 48 hours but no chest pain.

### Example 2: An Open-label, Pilot, Randomized Two-period Cross-Over Study to Assess the Food Effect on the 25 mg Tablet Formulation of Compound I at a Dose of 200 mg in Healthy Adult Volunteers

This example describes a clinical study for establishing, in healthy volunteers, the effect of a high fat, high caloric meal on the PK profile of Compound I, as compared to administration of the drug in the fasted state. The study also was intended to determine the safety and tolerability after a single oral dose of Compound I in the fed and fasted state in healthy volunteers. The measurements of PK, PD, and other clinical parameters were done as described in Example 1 above.

### Materials and Methods

### Study Design

This study was an open-label, randomized, two-period cross-over study in healthy volunteers aged 18-55 years. Subjects were screened up to 28 days before the first treatment period. Subjects were admitted to the clinical site on Day −1 (the day before dosing) of Period 1. Approximately half of the subjects randomly received a single dose of Compound I on Day 1 of the first treatment period after the ingestion of a high fat, high caloric breakfast, and the remainder were dosed in the fasted state. Any subject with a predose resting HR ≥95 beats per minute (bpm) was considered ineligible and was not treated. Any subjects with an acute gastrointestinal disorder which could impact drug/food absorption (e.g., vomiting, diarrhea) were rescheduled. Subjects were confined to the clinic until Day 4, and discharged after the 72-hour postdose PK and laboratory samples and vital signs were obtained. After a washout between dosing from 7 to 10 days (or, after consultation with the Investigator, up to 21 days after initial dosing if the subject was unable to attend within the 7 to 10-day window), the subject was admitted for Period 2. The sequence of fed/fasted versus fasted/fed periods was randomized. Subjects returned after the second treatment period for a safety follow-up visit on Day 7 (+1 day).

In both treatment periods, Compound I was administered with 240 mL of water. In the fasted state, the subjects fasted for 10 hours before and for 4 hours after the administration of Compound I. Water could have been ingested up to 1 hour before and after 1 hour post dosing. In the fed state, the subjects fasted for 10 hours before and for 4 hours after the ingestion of the meal, but could have ingested water up to 1 hour before and 1 hour after dosing. In the fed state, the subjects started ingesting the high fat, high caloric meal within 30 minutes prior to Compound I administration and finished the meal within 30 minutes. The meal contained approximately 800 to 1000 calories with about 50% of the calories from fat. The meal consisted of approximately 150 calories from protein, 250 calories from carbohydrate, and 500-600 calories from fat. An example of the meal was a breakfast consisting of two eggs fried in butter, two strips of bacon, two slices of buttered toast, 4 ounces of hash brown potatoes, and 8 ounces of whole milk.

### Treatments Administered

Each subject received two oral doses of 200 mg of Compound I formulated as 25 mg tablets (8 tablets) in a randomized, cross-over fashion, once in the fasted state and the other time after the ingestion of a high fat, high caloric breakfast. There was a washout of between 7 and 21 days between the administrations of the two doses. The Compound I drug substance was a crystalline, free-base, synthetic molecule with a molecular weight of 435.4. Compound I is nonhygroscopic and practically insoluble in aqueous media.

### Pharmacokinetic Assessments

Plasma drug concentrations were measured as described in Example 1 above. Blood samples to measure Compound I plasma concentration were collected at various time points, including on Day 1 predose (1 hour prior to dosing) and at 1 (±5 min), 2 (±5 min), 3 (±5 min), 4 (═10 min), 5 (±10 min), 6 (±10 min), 9 (±20 min), 12 (±20 min), 18 (±30 min), 24 (±30 min), 36 (±30 min), 48 (±30 min), and 72 (±30 min) hours postdose on both treatment periods.

### Electrocardiograms (12-Lead ECG)

ECG was performed as described in Example 1. The following intervals were measured: RR, PR, QRS, and QT. Heart rate (HR) was calculated as 60/(RRx1000) (with RR expressed in msec) and rounded to the nearest integer. Each individual ECG QT value was corrected for HR. The measured QT data was corrected for HR using the Fridericia method (QTcF) as per the following formulae/method (with QT, RR and QTc expressed in ms):

$$QTcF = \frac{QT}{{\frac{RR}{1000}}^{0.33}}$$

### Electrocardiogram Telemetry

Real-time telemetry ECG was displayed at various predetermined time points. Real-time telemetry ECG was displayed starting at least 1 hour predose and continuing through 48 hours postdose. The Investigator or designee monitored the continuous ECG telemetry data and correlated the finding(s) with any other clinical findings, study participant's medical history, study participant's clinical status and laboratory data to determine the clinical importance of the finding.

### Serum Troponin-I Concentrations

Serum troponin-I concentrations were determined as described in Example 1. Abnormal and/or rising troponin values (as per Investigator's judgment and taking into account potential Baseline troponin elevation) led to the subject being clinically evaluated for possible myocardial ischemia. If the subject had any signs or symptoms suggestive of possible cardiac ischemia, additional serial troponin (and other safety indicators such as creatine kinase MB isoenzyme [CK-MB]) levels were obtained, and continued dosing would be withheld until there was full understanding of the possible ischemic event. The entire clinical context would be evaluated (e.g., signs, symptoms, new ECG changes, new troponin, and CK-MB abnormalities) and correlated with any other relevant clinical findings, the subject's medical history, and laboratory data to determine the clinical significance of the findings.

### Study Results

### Plasma Concentrations of Compound I

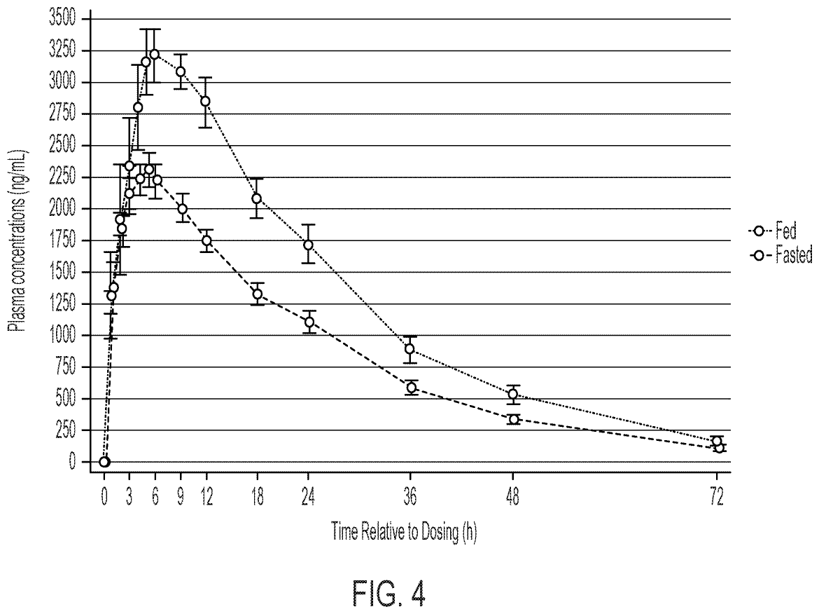

Plasma Compound I concentrations over time by fed/fasted status are summarized in Table 7 and FIG. 4. All randomized subjects (11 subjects) were given a single dose by oral administration of 200 mg Compound I following an overnight fast or a high fat meal. These 11 randomized subjects included 9 subjects who received treatment in both periods, 1 subject who received the study drug in the fed state, and 1 subject who received the study drug in the fasted state.

TABLE 7
Summary of Compound I Plasma Concentrations (ng/mL)*

| | | Fasted | | Fed | |
|---|---|---|---|---|---|
| Time Point | Statistic | (N = 10) | | (N = 10) | |
| Predose | n | 10 | | 10 | |
| | Mean (SD) | 0.000 | (0.000) | 0.092 | (0.291) |
| | Median (Min, Max) | 0.000 | (0, 0) | 0.000 | (0, 0.92) |
| | Geometric Mean (CV % of GM) | 0.000 | (CND) | 0.000 | (CND) |
| 1 hour postdose | n | 10 | | 10 | |
| | Mean (SD) | 1365 | (641.9) | 1315 | (1082) |
| | Median (Min, Max) | 1600 | (395, 2120) | 828.5 | (204, 3370) |
| | Geometric Mean (CV % of GM) | 1184 | (68.02) | 915.5 | (120.1) |
| 2 hours postdose | n | 10 | | 10 | |
| | Mean (SD) | 1828 | (415.3) | 1917 | (1383) |
| | Median (Min, Max) | 1825 | (1300, 2370) | 1160 | (437, 3860) |
| | Geometric Mean (CV % of GM) | 1785 | (23.57) | 1472 | (92.82) |
| 3 hours postdose | n | 10 | | 10 | |
| | Mean (SD) | 2122 | (408.2) | 2343 | (1233) |
| | Median (Min, Max) | 2180 | (1630, 2710) | 2335 | (646, 3750) |
| | Geometric Mean (CV % of GM) | 2086 | (19.82) | 1999 | (70.03) |
| 4 hours postdose | n | 10 | | 10 | |
| | Mean (SD) | 2224 | (400.3) | 2809 | (1070) |
| | Median (Min, Max) | 2285 | (1420, 2700) | 3030 | (1560, 4330) |
| | Geometric Mean (CV % of GM) | 2187 | (20.42) | 2613 | (42.87) |
| 5 hours postdose | n | 10 | | 10 | |
| | Mean (SD) | 2310 | (405.8) | 3151 | (827.6) |
| | Median (Min, Max) | 2405 | (1420, 2720) | 3345 | (1910, 4380) |
| | Geometric Mean (CV % of GM) | 2272 | (20.34) | 3044 | (29.15) |
| 6 hours postdose | n | 10 | | 10 | |
| | Mean (SD) | 2215 | (433.2) | 3204 | (638.0) |
| | Median (Min, Max) | 2320 | (1320, 2760) | 3325 | (2140, 4050) |
| | Geometric Mean (CV % of GM) | 2170 | (22.59) | 3142 | (21.62) |
| 9 hours postdose | n | 10 | | 10 | |
| | Mean (SD) | 2004 | (352.6) | 3080 | (427.4) |
| | Median (Min, Max) | 2040 | (1350, 2430) | 3220 | (2390, 3790) |
| | Geometric Mean (CV % of GM) | 1973 | (19.30) | 3053 | (14.29) |
| 12 hours postdose | n | 10 | | 10 | |
| | Mean (SD) | 1741 | (279.1) | 2841 | (639.2) |
| | Median (Min, Max) | 1800 | (1230, 2070) | 2725 | (2060, 4260) |
| | Geometric Mean (CV % of GM) | 1719 | (17.18) | 2781 | (21.70) |
| 18 hours postdose | n | 10 | | 10 | |
| | Mean (SD) | 1320 | (279.3) | 2082 | (504.6) |
| | Median (Min, Max) | 1355 | (903, 1630) | 2060 | (1290, 3210) |
| | Geometric Mean (CV % of GM) | 1292 | (22.35) | 2029 | (24.36) |
| 24 hours postdose | n | 10 | | 10 | |
| | Mean (SD) | 1099 | (276.8) | 1717 | (479.0) |
| | Median (Min, Max) | 1170 | (718, 1450) | 1670 | (938, 2720) |
| | Geometric Mean (CV % of GM) | 1066 | (27.22) | 1656 | (29.36) |
| 36 hours postdose | n | 10 | | 10 | |
| | Mean (SD) | 578.5 | (195.1) | 881.0 | (305.5) |
| | Median (Min, Max) | 596.5 | (309, 844) | 905.0 | (463, 1530) |
| | Geometric Mean (CV % of GM) | 545.8 | (38.51) | 834.4 | (36.25) |
| 48 hours postdose | n | 10 | | 10 | |
| | Mean (SD) | 331.2 | (126.3) | 525.5 | (234.3) |
| | Median (Min, Max) | 346.5 | (140, 516) | 549.0 | (209, 994) |
| | Geometric Mean (CV % of GM) | 305.8 | (46.67) | 475.9 | (51.60) |
| 72 hours postdose | n | 10 | | 10 | |
| | Mean (SD) | 108.5 | (54.61) | 160.6 | (107.3) |
| | Median (Min, Max) | 112.0 | (30.7, 188) | 143.0 | (43.4, 387) |
| | Geometric Mean (CV % of GM) | 93.15 | (69.76) | 129.1 | (83.17) |

*The lower limit of quantification (LLOQ) is 0.5. Concentrations below the LLOQ are set to zero (0). Eleven subjects received treatment; this included 9 subjects who received treatment in both periods, 1 subject who received study drug in the fed state, and 1 subject who received study drug in the fasted state. Abbreviations: CV % = percent of coefficient of variation; GM = geometric mean; CND = could not be determined; Max = maximum; Min = minimum; n = number of subjects with assessment at the timepoint being summarized; N = number of subjects in the PK population for the specified treatment; SD = standard deviation.

Plasma Compound I concentrations were detectable 1 to 72 hours postdose in all subjects in both the fed and fasted states. Mean plasma concentrations were higher in the fed state than the fasted state at 2 to 72 hours postdose, with Cₘₐₓ being 2310 (405.8) ng/mL and tₘₐₓ being 5 hours postdose in the fasted state and with Cₘₐₓ being 3204 (638.0) ng/ml and tₘₐₓ being 6 hours postdose in the fed state.

### Plasma Pharmacokinetic Parameters of Compound I

Plasma PK parameters for Compound I are summarized by treatment group in Table 8 below.

TABLE 8
Summary of Pharmacokinetic Parameters*

| | | Cₘₐₓ | Tₘₐₓ | AUCₗₐₛₜ | AUC_{inf} | T_{1/2, z}^{a} | MRT |
|---|---|---|---|---|---|---|---|
| Treatment | Statistic | (ng/mL) | (h) | (hr × ng/mL) | (hr × ng/mL) | (h) | (h) |
| Fasted | N | 10 | 10 | 10 | 10 | 10 | 10 |
| (N = 10) | Mean | 2347 | 4.700 | 60200 | 62580 | 14.28 | 22.55 |
| | (SD) | (366.9) | (1.059) | (13130) | (14310) | (2.107) | (3.300) |
| | Median | 2405 | 5.000 | 64350 | 66710 | 14.88 | 23.86 |
| | (Min, | (1630, | (3.000, | (39350, | (40240, | (10.60, | (17.40, |
| | Max) | 2760) | 6.000) | 74160) | 77720) | 16.58) | 27.18) |
| | Geometric | 2318 | 4.579 | 58810 | 60980 | 14.13 | 22.33 |
| | Mean | (17.23) | (25.35) | (23.65) | (24.90) | (15.72) | (15.36) |
| | (CV % of | | | | | | |
| | GM) | | | | | | |
| Fed | N | 10 | 10 | 10 | 10 | 10 | 10 |
| (N = 10) | Mean | 3677 | 6.900 | 89900 | 93480 | 13.82 | 23.08 |
| | (SD) | (500.7) | (3.695) | (17480) | (20070) | (2.833) | 4.644 |
| | Median | 3770 | 5.500 | 89310 | 93950 | 13.46 | 23.64 |
| | (Min, | (2650, | (2.000, | (65950, | (66620, | (10.17, | (16.35, |
| | Max) | 4380) | 12.00) | 126400) | 136500) | 18.08) | 30.51) |
| | Geometric | 3644 | 6.017 | 88400 | 91600 | 13.56 | 22.65 |
| | Mean | (14.55) | (61.81) | (19.52) | (21.45) | (20.81) | (20.52) |
| | (CV % of | | | | | | |
| | GM) | | | | | | |

*Abbreviations: AUC_{inf} = area under plasma concentration-time curve from time 0 to infinity; AUCₗₐₛₜ = area under the plasma concentration-time curve from time 0 up to the last measurable concentration; Cₘₐₓ = maximum observed plasma concentration; CV % = percent of coefficient of variation; GM = geometric mean; Max = maximum; Min = minimum; MRT = mean residence time; N = number of subjects in the PK population for the specified treatment; n = number of subjects with assessments for the parameter being summarized; PK = pharmacokinetic(s); T_{1/2, z} = apparent terminal phase elimination half-life; Tₘₐₓ = time of maximum observed plasma concentration. Concentrations below the lower limit of quantification were set to zero (0).
^{a}t_{1/2, z} is equivalent to t_{1/2}.

As shown in Table 8, following oral administration of a single 200 mg Compound I dose, exposure was approximately 50% higher (AUCₗₐₛₜ, AUC_{inf}) and 60% higher (Cₘₐₓ) in the fed state versus the fasted state. Mean (SD) maximum plasma concentration (Cₘₐₓ) was 2347 (366.9) ng/ml in the fasted state and 3677 (500.7) ng/mL in the fed state. Median (range) Tₘₐₓ occurred at 5 (3.0 to 6.0) hours in the fasted state and 5.5 (2.0 to 12.0) hours in the fed state.

To assess the effect of food on the PK of Compound I, the two one-sided t-test procedure was used to construct 90% CI around the geometric mean ratios (fed/fasted) of plasma AUC_{inf}, AUCₗₐₛₜ, and Cₘₐₓ. A mixed effects model with sequence, period, and treatment condition as fixed effects and subject as a random effect was used. Bioequivalence data are shown in Table 9 below for all subjects who received a single dose of 200 mg Compound I.

TABLE 9
Bioequivalence Assessment of PK Parameters (N = 11)*

| | | | Geometric | | |
|---|---|---|---|---|---|
| | | | Mean Ratio - | 90% CI | 90% CI |
| | Fasted | Fed | Fed/Fasted | Lower | Upper |
| Parameter | LSGM | LSGM | (% of Reference) | Bound¹ | Bound¹ |
| AUC_{inf} | 59400 | 91600 | 154.28 | 130.30 | 182.67 |
| (hr × ng/mL) | | | | | |
| AUCₗₐₛₜ | 57400 | 88400 | 154.02 | 131.11 | 180.92 |
| (hr × ng/mL) | | | | | |
| Cₘₐₓ (ng/mL) | 2300 | 3640 | 158.11 | 137.11 | 182.33 |

*Abbreviations: AUC_{inf} = area under the plasma concentration-time curve from time 0 to infinity; AUCₗₐₛₜ = the area under the plasma concentration-time profile from time 0 up to the last measurable plasma concentration; CI = confidence interval; Cₘₐₓ = maximum observed plasma concentration; LSGM = least squares geometric mean; N = number of subjects in the PK population for the specified treatment.
¹Absence of a food effect is concluded if the 90% CI for the ratio of geometric means based on log-transformed data is contained in the equivalence limits of 80-125%.

As shown above, the geometric mean ratios (fed/fasted) were 154.28%, 154.02%, and 158.11%, respectively, showing approximately 50% increases for AUC_{inf} and AUCₗₐₛₜ (i.e., AUC₀₋ₜ) and 60% increase for Cₘₐₓ, in the fed state. The 90% CI for the ratio of geometric means based on log-transformed data is not contained within the equivalence limits of 80-125% for AUC_{inf}, AUCₗₐₛₜ, and Cₘₐₓ, demonstrating a food effect.

Bioequivalence data are shown in Table 10 below for all subjects who completed both fasted and fed periods of Compound I.

TABLE 10
Bioequivalence Assessment of PK Parameters (N = 9)

| | | | Geometric | | |
|---|---|---|---|---|---|
| | | | Mean Ratio - | 90% CI | 90% CI |
| | Fasted | Fed | Fed/Fasted | Lower | Upper |
| Parameter | LSGM | LSGM | (% of Reference) | Bound¹ | Bound¹ |
| AUC_{inf} | 61100 | 93900 | 153.63 | 130.14 | 181.35 |
| (hr × ng/mL) | | | | | |
| AUCₗₐₛₜ | 59000 | 90500 | 153.20 | 130.94 | 179.25 |
| (hr × ng/mL) | | | | | |
| Cₘₐₓ (ng/mL) | 2370 | 3700 | 156.43 | 136.16 | 179.73 |

*Abbreviations: AUC_{inf} = area under the plasma concentration-time curve from time 0 to infinity; AUCₗₐₛₜ = the area under the plasma concentration-time profile from time 0 up to the last measurable plasma concentration; CI = confidence interval; Cₘₐₓ = maximum observed plasma concentration; LSGM = least squares geometric mean; N = number of subjects in the PK population for the specified treatment.
¹Absence of a food effect is concluded if the 90% CI for the ratio of geometric means based on log-transformed data is contained in the equivalence limits of 80-125%.

As shown above, the geometric mean ratios (fed/fasted) were 153.63%, 153.20%, and 156.43%, showing approximately 50% increases respectively for AUC_{inf}, AUC₀₋ₜ and Cₘₐₓ in the fed state. The 90% CI for the ratio of geometric means based on log-transformed data is not contained within the equivalence limits of 80-125% for AUC_{inf}, AUCₗₐₛₜ, and Cₘₐₓ, demonstrating a food effect.

### PK Conclusions

Following a single 200 mg dose, plasma Compound I was detectable 1 to 72 hours postdose in both the fed and fasted states. Concentrations peaked at 5 hours in the fasted state and at 5.5 hours in the fed state (Table 8). Exposure was 50% (based on AUCₗₐₛₜ, AUC_{inf}) to 60% (based on Cₘₐₓ) higher in the fed state versus the fasted state (Table 9, Table 10). In all subjects, the 90% CI for the ratio of geometric means based on log-transformed data was not contained within the equivalence limits of 80-125% for AUC_{inf}, AUCₗₐₛₜ, and Cₘₐₓ, demonstrating a food effect on Compound I PK. The same result was obtained when subjects who completed both fasted and fed periods were analyzed.

### Safety Evaluation

This study shows that overall, Compound I was well-tolerated at a single dose of 200 mg and no notable safety signals were identified during the study. All AEs were mild or moderate in severity and overall, most AEs were unrelated to study drug. There was no trend for increased frequency or severity of AEs with fasted vs. fed status. The most common (occurring in >2 subjects) AE was headache, which occurred in 4 subjects in the fasted state and 1 subject in the fed state. Cardiac disorders occurred in 2 subjects in the fasted state (1 sinus tachycardia and 1 ventricular tachycardia) and 1 subject in the fed state (palpitations); both AEs resolved and no action was taken with study treatment. The only drug-related AE occurring in more than 1 subject was headache (3 subjects in the fasted state and 1 subject in the fed state).

No increase in troponin-I was observed in any subject in either the fasted or fed state. There also were no clinically significant changes in safety laboratory parameters or vital signs, or in ECG intervals, in this study. There were 3 (30.0%) abnormal ECG results recorded in the fasted state, and 2 (20.0%) in the fed state.

### Example 3: Randomized, Double-blind, Placebo-controlled, Two-Part, Adaptive Design Study of Safety, Tolerability, Preliminary Pharmacokinetics, and Pharmacodynamics of Single and Multiple Ascending Oral Doses of Compound I in Patients with Stable HFrEF

This example describes a study to establish preliminary safety and tolerability of single- and multiple-ascending oral doses of Compound I in ambulatory patients with stable heart failure with reduced ejection fraction (HFrEF). Key eligibility criteria included stable HFrEF of ischemic or nonischemic origin, treated with guideline-directed medical therapy (EF initial requirement during Screening was 20 to 45%, and was later changed by amendment to 15 to 35%). Subjects with active ischemia or severe or valvular heart disease were excluded. The study also aimed (1) to establish preliminary human PK of Compound I after single- and multiple-ascending oral doses of Compound I in patients with HFrEF; (2) to determine changes in left ventricular stroke volume (LVSV) derived from left ventricular outflow tract-velocity time integral (LVOT-VTI), left ventricular ejection fraction (LVEF) and change in left ventricular fractional shortening (LVFS) with Compound I after ascending single and multiple doses compared with Baseline and placebo as measured by transthoracic echocardiography (TTE); (3) to determine changes in systolic ejection time (SET) with Compound I after ascending single and multiple doses compared with Baseline and placebo as measured by TTE; and (4) to determine changes in pharmacodynamics (PD) dose/concentration effects (change in LVSV (derived from LVOT-VTI), LVEF, LVFS) with Compound I compared with Baseline and placebo after ascending single and multiple doses, as measured by TTE.

The study also explored (1) the effect of Compound I on LV strain, LV dimensions, LV diastolic function, (2) potential electrocardiogramaart rate-corrected QT interval (QTc) effects with administration of Compound I, (3) the relationship between pharmacogenetic profile and PK-PD properties of Compound I, (4) potential impact of genetic etiology of dilated cardiomyopathy (DCM) on either PD or safety-related parameters, (5) the effect of Compound I on right ventricle (RV) contractility, (6) changes in SET with Compound I, during Part 1 of the study (single-ascending dosing [SAD]), using photoplethysmography, and (7) the plasma and/or urine concentrations and pharmacokinetics of metabolites of Compound I.

### Materials and Methods

### Study Design

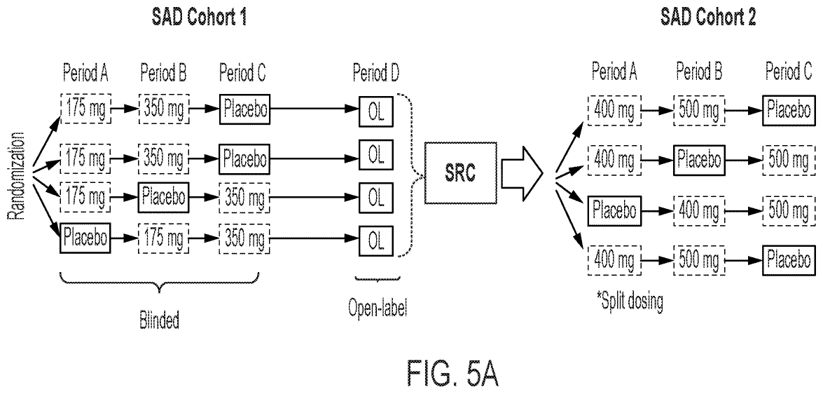

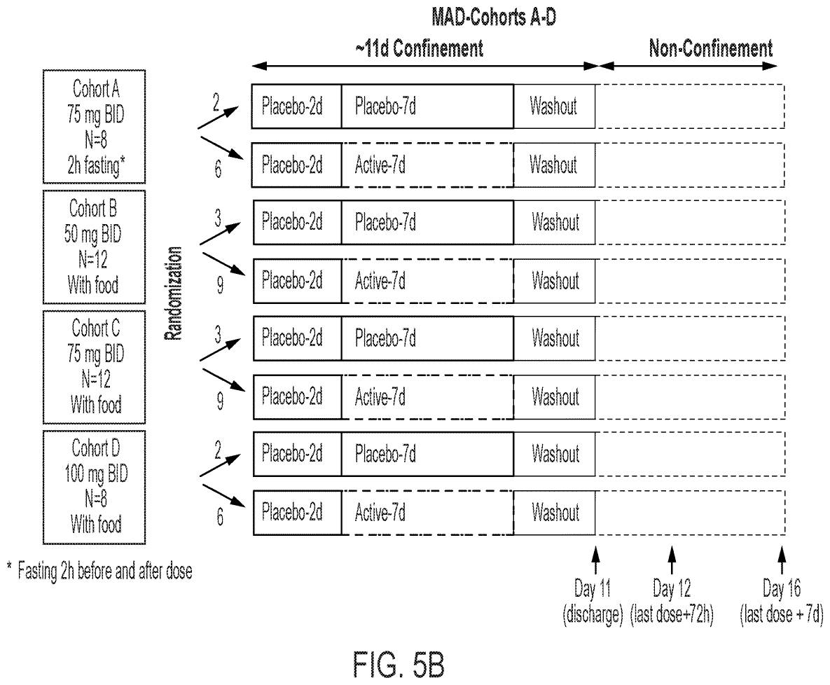

Part 1 of this two-part study evaluated single-ascending doses (SAD) of Compound I, and Part 2 evaluated multiple-ascending doses (MAD) of Compound I (FIGS. 5A and 5B).

### Part 1 (SAD Cohorts)

Part 1 was a randomized, crossover, DB, placebo-controlled, two-cohort, sequential ascending (oral) single-dose study in ambulatory patients with heart failure. All patients received placebo and 2 or 3 active doses of Compound I. Each patient underwent sequential, single-dose treatment events separated by no fewer than 5 days and no more than 14 days. Patients in Cohort 1 may also return for a fourth dosing period (open label) after the SRC reviews available data and recommends the dose. Patients enrolled prior to the implementation of Amendment 1 may be offered the opportunity to return for the open-label period. Patients in Cohort 2 participated in 2 to 4 dosing periods, based on SRC decision. Patients were randomized to one of the different dosing sequences outlined in FIG. 5A. Multiple patients could be dosed at the same time or during the same week depending on administrative issues, i.e., capacity and scheduling.

For each dosing period, patients were admitted to the clinical site on Day-1. Patients were assessed for absence of exclusion criteria (e.g., new lab abnormalities and/or conditions that indicate the patient is clinically unstable). They received Compound I or placebo in the morning of Day 1 followed by serial PK and PD assessments, as well as serial safety assessments. Patients were discharged on Day 3 (i.e., ˜48 hours following Day 1 dosing). An additional outpatient plasma PK sample was taken on the morning of Day 4 at 72 hours postdose.

Before administering a dose, all available safety data was reviewed, including vital signs, safety laboratory values including locally assayed troponin concentrations, TTEs, ECGs, and ECG telemetry. Dosing with DB treatment took place at the same time each of the dosing days. Background concomitant medications, including diuretic if applicable, was also administered at the same time each of the dosing days. Prior to dosing, any patient with a predose resting HR ≥95 bpm (mean of 3 measurements) was considered ineligible and not treated. A full PK profile and multiple TTEs and ECGs were obtained at Baseline and after each dose. Patients returned for a final safety Follow-up visit 7 days (±1 day) following the last dose. During the study, the patients continued to ingest their medications for the treatment of their congestive heart failure and other medical conditions at the same doses and as close to the same times as usual.

### Part 2 (MAD Cohorts)

This was a randomized, parallel-group, DB, placebo-controlled, adaptive design, sequential ascending (oral) multiple-dose study in stable patients with heart failure. Four MAD Cohorts (A, B, C, D) were enrolled (FIG. 5B). An SRC reviewed results from each cohort and determined the dose and confirmed initial sample size for the subsequent cohort. Additionally, the first 3 patients in each cohort had LVEF ≥25%; the SRC reviewed preliminary safety data from these patients and decided whether to open cohort enrollment to patients with LVEF <25%.

After Screening and qualification, patients were confined to a clinical testing facility from Day 1 (Check-in) to Day 11. Each patient initially received placebo BID for 2 days (Days 1 and 2) in single-blinded manner (“run-in” during acclimatization to confinement in the Clinical Testing Unit) prior to receiving the randomized DB study drug treatment on Day 3. All patients then received either placebo or active Compound I for 7 days (Days 3 through 9), with a follow-up period with patients discharged from the unit on Day 11. A final follow-up clinic visit was conducted on Day 16. More than one patient could be dosed in a cohort at the same time or during the same week depending on administrative issues, i.e., capacity and scheduling.

Patients were dosed twice daily (every 12 hours). Doses could occur ±2 hours from scheduled dosing times as long as doses were separated by at least 10 hours and by no more than 14 hours. The exception to the twice daily dosing was on Day 9 (last dose of randomized DB study drug treatment). On Day 9, a single morning dose was administered.

Before each dosing event, all available safety data from the previous days was reviewed (for non-confined patients, if a home health nurse was utilized, the nurse and site were in daily communication to ensure safety). Dosing of DB treatment took place at approximately the same time each day.

During the study, multiple evaluations were performed that included: serial TTE assessments (11-14 TTEs per patient on Days 1, 2, 3, 4, 7, 9, 10 and 11); PK sampling (PK sample collected concomitantly with every post-randomization echocardiogram); ECGs (on Days 2, 3, 4, 7, 9, 10, 11 and 16); troponin (collected concomitantly with every post-randomization ECG); and safety laboratory assessments. Confined patients underwent continuous telemetry. Holter monitoring was performed in all patients at baseline (Days 1-2) and at the end of double-blind treatment (Days 7-9). Vital signs were collected daily.

### Inclusion Criteria

This study was performed in patients with HFrEF due to any etiology. Each patient met at least the following criteria to be included in this study:
1. Men or women 18 to 80 years of age at the Screening visit
2. Body mass index (BMI) 18 to 40 kg/m², inclusive, at the Screening visit and all required assessments can be reliably performed
3. Sinus rhythm or stable atrial pacing with mean resting HR 50-95 beats per minute (bpm), inclusive (Patient will be ineligible to dose if, on Day 1, the predose HR measurement is ≥95 bpm. Heart rate is the mean of 3 measurements taken 1 minute apart. A single measurement would not make a patient ineligible.
4. Has stable, chronic HFrEF of moderate severity as defined by all of the following:
   (i) For the first 3 patients in each MAD Cohort testing a new (higher) daily dose: documented LVEF 25% to 35% during Screening (as confirmed by ECHO Central Lab)
   (ii) For other patients in the MAD Cohorts (and all patients in SAD Cohorts): documented LVEF 15% to 35% during Screening (as confirmed by ECHO Central Lab)
   (iii) LVEF must be confirmed with second screening ECHO to be performed at least 7 days after initial screening ECHO. Results of both must meet inclusion criteria and must be received from core lab prior to dosing. In the event of extended screening windows due to SRC reviews, effort should be made to ensure second ECHO is near planned time of randomization
   (iv) Chronic medication for the treatment of heart failure consistent with current guidelines that has been given at stable doses for ≥2 weeks with no plan to modify during the study. This includes treatment with at least one of the following unless not tolerated or contraindicated: beta-blocker, angiotensin converting enzyme (ACE) inhibitor/angiotensin receptor blocker (ARB)/angiotensin receptor neprilysin inhibitor (ARNI).

### Exclusion Criteria

Patients who met any of the following criteria were excluded from the study:
1. Inadequate echocardiographic acoustic windows
2. Any of the following ECG abnormalities: (a) QTcF >480 ms (Fridericia's correction, not attributable to pacing or prolonged QRS duration, average of triplicate Screening ECGs) or (b) second-degree atrioventricular block type II or higher in a patient who has no pacemaker
3. Hypersensitivity to Compound I or any of the components of the Compound I formulation
4. Active infection as indicated clinically as determined by the investigator
5. History of malignancy of any type within 5 years prior to Screening, with the exception of the following surgically excised cancers occurring more than 2 years prior to Screening: in situ cervical cancer, nonmelanomatous skin cancers, ductal carcinoma in situ, and nonmetastatic prostate cancer
6. Positive serologic test at Screening for infection with human immunodeficiency virus (HIV), hepatitis C virus (HCV), or hepatitis B virus (HBV)
7. Hepatic impairment (defined as alanine aminotransferase (ALT)/aspartate aminotransferase (AST)>3 times ULN and/or total bilirubin (TBL)>2 times ULN)
8. Severe renal insufficiency (defined as current estimated glomerular filtration rate [eGFR] <30 mL/min/1.73 m² by simplified Modification of Diet in Renal Disease equation [sMDRD])
9. Serum potassium <3.5 or >5.5 mEq/L
10. Any persistent out-of-range safety laboratory parameters (chemistry, hematology, urinalysis), considered by the investigator and medical monitor to be clinically significant
11. History or evidence of any other clinically significant disorder, condition, or disease (including substance abuse) that would pose a risk to patient safety or interfere with the study evaluation, procedures, or completion, or lead to premature withdrawal from the study
12. Participated in a clinical trial in which the patient received any investigational drug (or is currently using an investigational device) within 30 days prior to Screening, or at least 5 times the respective elimination half life (whichever is longer)
13. At Screening, symptomatic hypotension, or systolic BP >170 mmHg or <90 mmHg, or diastolic BP >95 mmHg, or HR <50 bpm. HR and BP will be the mean of 3 measurements taken at least 1 minute apart.
14. Current angina pectoris
15. Recent (<90 days) acute coronary syndrome
16. Coronary revascularization (percutaneous coronary intervention [PCI] or coronary artery bypass graft [CABG]) within the prior 3 months
17. Recent (<90 days) hospitalization for heart failure, use of chronic IV inotropic therapy or other cardiovascular event (e.g., cerebrovascular accident)
18. Uncorrected severe valvular disease
19. Elevated Troponin I (>0.15 ng/mL) at Screening, based on Central Laboratory assessments. Note: Central Laboratory Troponin I assay ULN is 0.03 ng/ml
20. Presence of disqualifying cardiac rhythms that would preclude study ECG or echocardiographic assessments, including: (a) Current atrial fibrillation, (b) recent (<2 weeks) persistent atrial fibrillation, or (c) frequent premature ventricular contractions. Patients with active cardiac resynchronization therapy (CRT) or pacemaker (PM) are eligible if initiated at least 2 months prior with no plan to change CRT or PM settings during the study.
21. A life expectancy of <6 months.

### Study Treatment

In Part 1 (SAD), study patients received separate ascending doses of Compound I (2 to 3 doses) and a single dose of matching placebo. In Part 2 (MAD), study patients received single-blind placebo BID for Days 1 and 2 and then received DB treatment (either placebo or Compound I) for 7 days (Days 3 through 9). In Cohorts A, B, C, and D, on Day 9 patients received a single dose of placebo or Compound I in the morning for serial PK/PD assessments, while on Days 3 through 8 patients in these cohorts received placebo or Compound I BID.

Compound I drug substance was as described in Example 1 above and was provided as 5, 25, or 100 mg tablets. Placebo tablets were provided as matching tablets. The tablets were blistered and then carded. Each blister card contained only 5 mg, only 25 mg, only 100 mg, or only placebo. The blister cards were packaged into “Kit Boxes.”

### Study Medication, Administration, and Schedule

Study medication consisted of Compound I 5 mg tablets, 25 mg tablets, 100 mg tablets, or matching placebo tablets. In Part 1 (SAD), Compound I or placebo was administered after an overnight fast (at least 6 hours), while in Part 2 (MAD), Compound I was administered after a 2 hour fast (Cohort A) or with food (Cohorts B, C, and D). The dose was ingested with a minimum of 240 mL of water, but more water was ingested as needed. The entire dose was administered over a period of up to 15 minutes. The time of dose used to determine future assessments was the time the last tablet was taken. In the cohorts for Part 2 (MAD), a BID regimen was used.

In Part 1 (SAD), patients fasted overnight (approximately 6 hours) through 4 hours postdose. With the exception of the water consumed with the dose, water could be ingested until approximately 1 hour prior to dosing and approximately 1 hour after dosing. If doses were split, subjects fasted 6 hours prior to the first half-dose. A light, low-fat snack could be consumed 2 hours after the first half-dose and a fast continued through 2 hours after the second half-dose.

In Part 2 (MAD), Cohort A patients fasted for 2 hours before and 2 hours after dosing. For example, if morning dosing occurred at 8 AM, patients could have a snack at 6 AM and a full breakfast at 10 AM. If afternoon dosing occurred at 8 PM, patients could have dinner at 6 PM and a snack at 10 PM. These times could be adjusted based on local scheduling preferences, but doses were separated by at least 10.5 hours. Cohort B, C, and D patients ingested food with each dose.

### Management of an Exaggerated Pharmacological Effect and Overdose

Based on the nonclinical pharmacological characteristics, exaggerated effects of Compound I could lead to myocardial ischemia. The duration of effect would follow the PK profile of Compound I with a Tₘₐₓ of 4 to 6 hours and a half-life of about 15 hours in healthy volunteers, but a slightly longer half-life in patients that received Compound I as part of Cohort 1 (20 to 25 hours). The clinical signs and symptoms, which could include chest pain, lightheadedness, diaphoresis, and ECG changes should start to abate over a short period of time. Any patient with signs and/or symptoms that may be secondary to cardiac ischemia was immediately evaluated by the physician for the possibility of cardiac ischemia and additional ECGs and serial troponins obtained as part of the evaluation as appropriate.

If evidence of cardiac ischemia was present, then the patient received standard therapy for ischemia as appropriate, including supplemental oxygen and nitrates. Caution in the administration of agents that increase HR was required, as Compound I may prolong the SET, which would result in decreasing the diastolic duration resulting in a decrease in diastolic ventricular filling. In addition, the exaggerated pharmacological effect could increase myocardial oxygen demand, so agents that might increase myocardial oxygen demand further were administered with caution.

Patients who received a greater dose than planned were supported as appropriate, such as described above if there is an exaggerated pharmacologic effect.

### Concomitant Therapy

During the study, the patients continued to ingest their medications for the treatment of their congestive heart failure and other medical conditions at the same doses and as close to the same times as usual, in order to maintain as best as possible similar preload and afterload conditions throughout the study to minimize confounding factors for the assessment of the effects of Compound I. In particular, if the patient was treated with diuretics, the time of administration of the diuretic relative to DB treatment was kept similar throughout the study. Times of administration of diuretics, if applicable, were collected. If the patient was not confined, the patient was instructed to maintain constant timing of daily administration of medications, including diuretics if applicable, and to record the time of administration.

All prescription and over-the-counter medications were reviewed by the investigator. Questions concerning enrollment or medications were discussed with the medical monitor. Over-the-counter medications could be taken at stable doses throughout the study (at investigator's discretion), and in amounts no greater than as directed per the label. All concomitant treatments (prescription or over-the-counter) were recorded. Other investigational therapies were discontinued at least 30 days prior to Screening or 5 half-lives (whichever is longer).

If the patient had an AE requiring treatment (including the ingestion of acetaminophen or ibuprofen), the medication was recorded; including time of the administration (start/stop), date, dose, and indication.

### PD Assessment

PD assessment was done by transthoracic echocardiography as described in Example 1 above. TTE evaluations of LVSV (derived from LVOT-VTI), LVEF, LVFS, SET, and other parameters were PD assessments at predetermined time points. The patients were on bed rest for 10 minutes before the TTEs were obtained. In Part 2 (MAD), TTEs were usually obtained before the morning dose and/or at 7 hours postdose (i.e., close to the anticipated peak effect based on the PK profile from the healthy volunteer studies).

### Safety and Efficacy Assessment

Safety and efficacy assessments were conducted by measuring patients' vital signs and laboratory parameters; performing TTE to measure, e.g., systolic ejection time; performing electrocardiograms (e.g., 12-lead ECG), real-time ECG telemetry (e.g., at least 3-lead), and Holter ECG; and measuring levels of troponin (e.g., troponin I and/or troponin T) and 4β-hydroxycholesterol.

The following safety laboratory parameters were measured: (1) hematology parameters (CBC, including differential count, and platelet count); (2) serum chemistry parameters (e.g., sodium, potassium, chloride, bicarbonate, calcium, magnesium, urea, creatinine, ALP, ALT, AST, total bilirubin, glucose, and CPK); and (3) urinalysis parameters (e.g., pH, protein, glucose, leukocyte cstcrasc, and blood).

Abnormal and/or rising troponin values (as per investigator's judgment and taking into account potential baseline troponin elevation frequently observed in heart failure) led to the patient being clinically evaluated for possible myocardial ischemia. Also, if the patient had any signs or symptoms suggestive of possible cardiac ischemia, additional serial troponin (and other safety labs, including creatine kinase-MB [CK-MB] samples) were obtained and continued dosing withheld until there was full understanding of the possible ischemic event. The entire clinical context (e.g., signs, symptoms, new ECG changes, new troponin, and CK-MB abnormalities) was evaluated and correlated with any other relevant clinical findings, patient's medical history, and laboratory data to determine the clinical significance of the findings. Troponin results performed on Day 2 of Part 1 (SAD) and Day 10 of Part 2 (MAD) at a local lab were reviewed prior to the patient being discharged the next day.

### Study Endpoints

Primary endpoints for this study (safety measures) included the following: treatment-emergent AEs and SAEs; ECG recordings, interpretation, and intervals; vital signs; serum Troponin I concentrations; laboratory abnormalities; and physical examination abnormalities.

The following were secondary endpoints:
1. The human PK profile of Compound I. The analysis included at a minimum the following PK parameters: Cₘₐₓ for each dose level, Tₘₐₓ for each dose level, AUC for each dose level dose, apparent first-order terminal elimination half-life (t_{1/2}), mean residence time (MRT) for each dose level, and accumulation ratios determined (with the appropriate confidence intervals) for Cₘₐₓ and AUC₀₋ₜ (Part 2 only).
2. SET as determined using TTE. The main parameters were the change from Baseline at each timepoint by treatment levels and the maximum change from Baseline.
3. The following as assessed by TTE: change from Baseline in LVSV (derived from LVOT-VTI), change from Baseline in LVEF, change from Baseline in LVFS, and change from Baseline in SET.

Exploratory endpoints were:
1. Pharmacokinetic dose proportionality of AUC and Cₘₐₓ after both single dose (Part 1) and multiple dose (Part 2)
2. To explore the potential effects of Compound I on QT interval, corrected using Fridericia's formula (QTcF), change from Baseline (either absolute or percent relative change), and if there is an effect, on the concentration effect relationship of changes from Baseline of QTcF
3. The relationship between Compound I plasma concentrations/PK parameters and PD parameters (LVEF, SET, LVFS, LVSV)
4. The following as assessed by TTE: change from Baseline in LV strain, change from Baseline in LV dimension, change from Baseline in LV diastolic function, change from Baseline in RV contractility, and change from Baseline in PEP (in Part 1)
5. SET, as assessed by photoplethysmography (in Part 1 only).

Additional possible endpoints were:
1. To explore genetic biomarkers and effect on the PK or PD profile of Compound I
2. Determination of Compound I metabolites in plasma samples
3. Amount of Compound I excreted in the urine for each of the collection intervals along with the total amount and the amount of the administered dose excreted into the urine.

### Study Results

PK/PD and Safety Data from Part 1 (SAD)—Cohorts 1 and 2

### Cohort 1

Eight patients with stable heart failure were enrolled and randomized to receive Compound I or placebo at a dose of 175, 350, 525, 450 (split dose), or 550 mg (split dose) in a crossover study design with four periods (A-D). All patients had heart failure with a nonischemic etiology and a mean Baseline ejection fraction of 43%. All eight subjects received placebo, 175 mg, and 350 mg (in random sequence) during Periods A to C. Six subjects elected to continue into a fourth open-label Period D, and doses received included: 350 mg (n=1), 525 mg (n=2), 450 mg (divided into 2 aliquots; n=1) and 550 mg (divided into 2 aliquots, n=2). The single doses were administered to patients under fasted conditions. The split doses were given four hours apart with patients fasting six hours prior to the first half-dose and 2 hours after the second half-dose, with a light snack allowed 2 hours after the first half-dose. Subsequently, patients underwent extended observation, followed by a washout period. This process was repeated until each patient had received at least three doses (Compound I or placebo).

### Cohort 2

Four subjects with stable heart failure were enrolled and randomized to receive Compound I or placebo at a dose of 400 mg (split dose) or 500 mg (split dose) over three periods (A-C). The split doses were given four hours apart with patients fasting six hours prior to the first half-dose and 2 hours after the second half-dose, with a light snack allowed 2 hours after the first half-dose. All four subjects received placebo, 400 mg, and 500 mg (in random sequence) during Periods A-C.

The results of the PK assessments are summarized below and in Table 11.

TABLE 11
Summary of Pharmacokinetic Parameters after
Oral Administration of Single Ascending Doses
to HFrEF Patients in SAD Cohorts 1 and 2

| | | | | | |
|---|---|---|---|---|---|
| | Cmax | | AUC0-24 | AUC0-∞ | |
| | (Min, Max) | | (hr × | (hr × | |
| | [CV %] | Tmax | ng/mL) | ng/mL) | t½ |
| Treatment | (ng/mL) | (h) (SD) | (SD) | (SD) | (h) (SD) |

SAD Cohort 1

| | | | | | |
|---|---|---|---|---|---|
| 175 mg | 1510 | 4.93 | 27,000 | 53,800 | 22.0 |
| (n = 8) | (1020, 2200) | (1.39) | (6070) | (13,800) | (4.40) |
| | [22.7] | | | | |
| 350 mg | 2760 | 6.15 | 50,500 | 103,000 | 21.0 |
| (n = 8) | (1800, 4530) | (2.00) | (13,700) | (27,200) | (3.23) |
| | [29.5] | | | | |
| 525 mg | 2720 | 5.74 | 54,000 | 127,000 | 24.7 |
| (n = 2) | (2630, 2810) | (0.48) | (2470) | (20,100) | (10.76) |
| | [4.68] | | | | |
| Split dose 450 | 4420 | 12.02 | 79,200 | 235,000 | 30.6 |
| mg (n = 1) | | | | | |
| Split dose 550 | 5280 | 8.93 | 97,900 | 213,000 | 21.5 |
| mg (n = 2) | (4930, 5620) | (1.41) | (9840) | (35,800) | (0.30) |
| | [9.28] | | | | |
| 4^{th} dose | 3590 | 4.05 | 70,100 | — | — |
| 350 mg (n = 1) | | | | | |

SAD Cohort 2

| | | | | | |
|---|---|---|---|---|---|
| 400 mg | 5455 | 9.0 | 161,500 | 191,600 | 24.1 |
| split dose | (4050, 6740) | (2.58) | (21,710) | (42,940) | (11.14) |
| (n = 4) | [1290] | | | | |
| 500 mg | 5883 | 8.5 | 190,700 | 231,100 | 24.0 |
| split dose | (4250, 7920) | (1.73) | (20,440) | (68,440) | (14.15) |
| (n = 4) | [1517] | | | | |

Abbreviations: AUC₀₋₂₄ = area under the plasma concentration-time curve from 0 to 24 hours; AUC_{0-∞} = area under the plasma concentration-time curve from 0 to infinity; Cₘₐₓ = maximum observed plasma concentration; CV = coefficient of variance; Max = maximum; Min = minimum; SAD = single-ascending dose; SD = standard deviation; t_{1/2} = apparent terminal elimination half-life; Tₘₐₓ = time of maximum observed plasma concentration.
Split dosing was the total dose divided evenly into 2 aliquots given 4 hours apart.

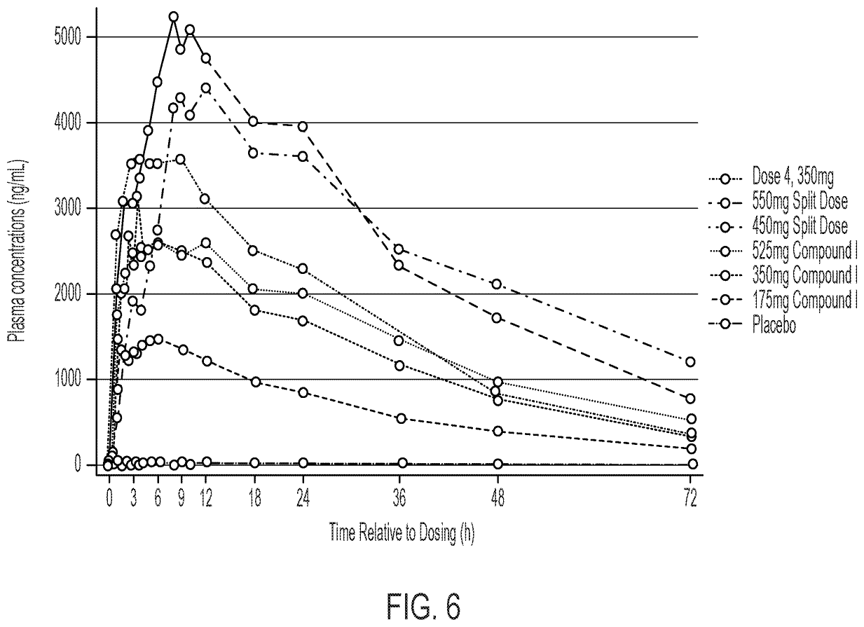

Mean plasma concentration-time profiles of Compound I for SAD Cohort 1 are depicted in FIG. 6. In this cohort, Compound I was detectable in all subjects that received Compound I at 72 hours post-dose. Compound I was also observed in plasma in four subjects who received placebo in Period B or C, indicating that Compound I was not eliminated completely within the washout period. The peak plasma concentration occurred at approximately 5 to 6 hours, ranging from 2.0 to 9.1 hours, following oral administration of a 175, 350, or 525 mg single dose of Compound I. The plasma exposure (Cₘₐₓ, AUC₀₋₂₄, and AUC_{0-∞}) increased with increasing Compound I dose in a nearly dose-proportional manner for single doses from 175 mg to 350 mg but reached a plateau in Cₘₐₓ and increased less than dose-proportionally in AUC for the 525 mg dose. The mean (SD) Cₘₐₓ was 1510 (350) ng/mL for the 175 mg single dose, 2760 (856) ng/mL for the 350 mg single dose, and 2720 (127) ng/mL for the 525 mg single dose. The mean (SD) AUC_{0-∞} was 53800 (13800) ng*h/mL for the 175 mg single dose, 103000 (27200) ng*h/mL for the 350 mg single dose, and 127000 (20100) ng*h/mL for the 525 mg single dose.

These results were comparable to those observed in healthy subjects as described previously in Example 1. The decreased exposure from 525 mg dosing likely resulted from reduced bioavailability due to poor solubility, slow dissolution, and incomplete absorption of undissolved drug molecules in the gastrointestinal tract. In order to overcome the saturable absorption at high doses, split doses were given four hours apart to patients who had completed treatment in Period A, B, or C administered with placebo, 175 mg, or 350 mg single dose. One patient received a 450 mg dose and 2 patients received a 550 mg dose (split into two equal aliquots dosed at 4 hours apart) in Period D. As shown in Table 11, the exposure of Compound I after oral administration of 450 and 550 mg via split dose was increased in a more than dose-proportional manner compared to 175 mg and 350 mg single doses. The more than dose-proportional increase in exposure possibly resulted from food intake between the two doses.

For both Cohorts 1 and 2, the pharmacodynamic effects of Compound I on echocardiographic markers of cardiac structure and function were analyzed by Compound I plasma concentration groups: <2000 ng/ml (low concentration group) and ≥2000 ng/ml (high concentration group) (Table 12).

In the high plasma concentration group (≥2000 ng/mL), Compound I was associated with a statistically significant increase from baseline in mean (SE) stroke volume (9.0 [3.0] ml; p<0.001) and in mean (SE) LV ejection fraction (4.4% [1.9]; p<0.05) as well as with a significant decrease in mean (SE) LV global longitudinal strain (−2.1% [0.7]; p<0.001).

Administration of Compound I resulted in approximately 10% relative increases from baseline in cardiac contractility across multiple echocardiographic measures, including stroke volume (SV), LVEF, and fractional shortening (FS). In increasing the heart's contractility, Compound I did not appear to meaningfully change duration of the contraction or the heart's ability to relax and fill with oxygenated blood. A modest increase in SET was seen (<50 msec) and the impact of Compound I on left ventricular filling was minor across multiple measures of diastolic relaxation. These data, as summarized in Table 12, were consistent with results provided in Example 1 in healthy volunteers.

TABLE 12
Change from Baseline (Placebo-Corrected) in Selected Transthoracic
Echocardiography Parameters by Compound I Plasma Concentration
Group (Pooled SAD Cohorts 1 and 2)

| | Mean change (SE)^{b, c} by Compound I | |
|---|---|---|
| | plasma concentration group | |
| Baseline^{a} | <2000 ng/mL | ≥2000 ng/mL |
| (n = 12) | (n = 7) | (n = 12) |

Plasma concentration (ng/mL)

| | | | | | |
|---|---|---|---|---|---|
| Mean (SD) | — | 1390 | (276) | 3823 | (1426) |
| Median (range) | — | 1307 | (951-1870) | 3795 | (2010-7500) |

Measures of LV systolic function

| | | | | | | |
|---|---|---|---|---|---|---|
| LVSV (mL) | 74 | (15) | 1.0 | (3.7) | 9.0 | (3.0)** |
| LVEF (%) | 41 | (7.5) | 4.1 | (2.3) | 4.4 | (1.9)* |
| LVFS (%) | 22 | (3.7) | 3.1 | (1.4)* | 2.8 | (1.1)* |
| SET (ms) | 299 | (28) | 8 | (10) | 36 | (8)** |
| LVGLS (%) | −12 | (3.3) | −1.1 | (0.9) | −2.1 | (0.7)** |

LV dimensions and volumes

| | | | | | | |
|---|---|---|---|---|---|---|
| LVESD (mm) | 42 | (4.0) | −1.3 | (1.2) | −1.8 | (1.0) |
| LVEDD (mm) | 54 | (4.3) | 0.2 | (1.3) | −0.3 | (1.1) |
| LVESV (mL) | 40 | (10) | −4.0 | (2.4) | −3.5 | (2.0) |
| LVEDV (mL) | 67 | (11) | −2.5 | (3.2) | −1.9 | (2.7) |

Relaxation/diastolic function

| | | | | | | |
|---|---|---|---|---|---|---|
| e′ lateral | 7.4 | (3.0) | −1.0 | (1.2) | −1.0 | (1.1) |
| (m/sec) | | | | | | |
| E/e′ lateral | 15 | (19) | 0.6 | (1.5) | 0.2 | (1.2) |
| E wave peak | 83 | (39) | −1.1 | (4.6) | −1.6 | (4.3) |
| (cm/s) | | | | | | |
| A wave peak | 87 | (36) | −3.2 | (8.8) | −8.8 | (7.5) |
| (cm/s) | | | | | | |
| E/A ratio | 1.4 | (1.2) | 0.09 | (0.11) | 0.08 | (0.09) |

A, late peak wave velocity from mitral inflow Doppler; e′, peak atrioventricular valve annular velocity in early diastole; E, early peak wave velocity from mitral inflow Doppler; IVRT, isovolumic relaxation time; LS, least-squares; LV, left ventricular; LVEDD, left ventricular end-diastolic diameter; LVEDV, left ventricular end-diastolic volume; LVEF, left ventricular ejection fraction; LVESD, left ventricular end-systolic diameter; LVESV, left ventricular end-systolic volume; LVFS, left ventricular fractional shortening; LVGLS, left ventricular global longitudinal strain; LVSV, left ventricular stroke volume; SD, standard deviation; SE, standard error; SET, systolic ejection time; TTE, transthoracic echocardiogram.
For the analysis, all assessments are included in the column corresponding to the Compound I concentration reached concomitantly to the assessments. As a result, 7 patients contributed to the low (<2,000 ng/mL) Compound I concentration group only and 12 patients contributed to both the low and high (≥2,000 ng/mL) Compound I concentration groups.
^{a}Absolute arithmetic mean values and SD for the baseline measurement for all Compound I-treated patients, excluding patients receiving placebo.
^{b}LS mean difference (SE) between each plasma concentration group (<2000 ng/mL or ≥2000 ng/mL) and placebo (concentration = 0) in TTE parameters' change from baseline.
^{c}SE of LS mean difference = SE of the LS mean difference.
*p < 0.05.
**p < 0.01.

Single-ascending doses of Compound I administered in HFrEF patients in the range of 175 to 550 mg (across both SAD Cohorts 1 and 2) were safe and generally well-tolerated. There were no serious AEs, TEAEs of severe intensity, or TEAEs leading to study discontinuation. The list of observed TEAEs reported is shown in Table 13. No TEAE occurred in more than 1 subject and all observed TEAEs were either mild or not considered related to study drug (with the exception of TEAEs observed in one subject at the highest dose of 550 mg, which are described below in more detail).

TABLE 13
TEAEs Observed in SAD Cohorts 1 and 2

| | | | | 400 mg | 450 mg | 500 mg | | 550 mg | |
|---|---|---|---|---|---|---|---|---|---|
| | | | | Split | Split | Split | | Split | Total |
| | Placebo | 175 mg | 350 mg | Dose | Dose | Dose | 525 mg | Dose | Active |
| | (n = 12) | (n = 8) | (n = 8) | (n = 4) | (n = 1) | (n = 4) | (n = 2) | (n = 2) | (n = 12) |
| SOC, PT | n (%) | n (%) | n (%) | n (%) | n (%) | n (%) | n (%) | n (%) | n (%) |
| Any SOC, | 3 (25.0) | 2 (25.0) | 5 (62.5) | 3 (75.0) | — | — | — | 1 (50.0) | 10 (83.3) |
| any PT | | | | | | | | | |

Blood and lymphatic

| | | | | | | | | | |
|---|---|---|---|---|---|---|---|---|---|
| Anemia | — | — | — | 1 (25.0) | — | — | — | — | 1 (8.3) |

Cardiac

| | | | | | | | | | |
|---|---|---|---|---|---|---|---|---|---|
| Cardiac | — | — | — | — | — | — | — | 1 (50.0) | 1 (8.3) |
| discomfort | | | | | | | | | |
| Ventricular | — | — | — | 1 (25.0) | — | — | — | — | 1 (8.3) |
| extrasystoles | | | | | | | | | |

Gastrointestinal

| | | | | | | | | | |
|---|---|---|---|---|---|---|---|---|---|
| Diarrhoea | 1 (8.3) | — | 1 (12.5) | — | — | — | — | — | 1 (8.3) |
| Nausea | 1 (8.3) | — | — | — | — | — | — | — | — |
| Oral | — | — | 1 (12.5) | — | — | — | — | — | 1 (8.3) |
| contusion | | | | | | | | | |

General disorders and administration site conditions

| | | | | | | | | | |
|---|---|---|---|---|---|---|---|---|---|
| Infusion site | — | 1 (12.5) | — | — | — | — | — | — | 1 (8.3) |
| discomfort | | | | | | | | | |
| Fatigue | 1 (8.3) | 1 (12.5) | — | — | — | — | — | — | 1 (8.3) |

Infections and infestations

| | | | | | | | | | |
|---|---|---|---|---|---|---|---|---|---|
| Diverticu- | 1 (8.3) | — | — | — | — | — | — | — | 1 (8.3)− |
| litis | | | | | | | | | |
| Urinary tract | — | 1 (12.5) | — | — | — | — | — | — | 1 (8.3) |
| infection | | | | | | | | | |

Investigations

| | | | | | | | | | |
|---|---|---|---|---|---|---|---|---|---|
| Troponin | — | — | — | — | — | — | — | 1 (50.0) | 1 (8.3) |
| increase | | | | | | | | | |

Metabolism and nutrition

| | | | | | | | | | |
|---|---|---|---|---|---|---|---|---|---|
| Hypomag- | — | — | 1 (12.5) | — | — | — | — | — | 1 (8.3) |
| nesemia | | | | | | | | | |

Musculoskeletal and connective tissue

| | | | | | | | | | |
|---|---|---|---|---|---|---|---|---|---|
| Bursitis | — | — | 1 (12.5) | — | — | — | — | — | 1 (8.3) |
| Neck pain | — | — | — | 1 (25.0) | — | — | — | — | 1 (8.3) |

Nervous system

| | | | | | | | | | |
|---|---|---|---|---|---|---|---|---|---|
| Dizziness | 1 (8.3) | — | — | — | — | — | — | — | — |
| Headache | 1 (8.3) | — | — | — | — | — | — | — | — |

Renal and urinary

| | | | | | | | | | |
|---|---|---|---|---|---|---|---|---|---|
| Proteinuria | — | — | 1 (12.5) | — | — | — | — | — | 1 (8.3) |

Respiratory, thoracic, and mediastinal

| | | | | | | | | | |
|---|---|---|---|---|---|---|---|---|---|
| Dyspnea | 1 (8.3) | 1 (12.5) | — | — | — | — | — | 1 (50.0) | 1 (8.3) |

Vascular

| | | | | | | | | | |
|---|---|---|---|---|---|---|---|---|---|
| Orthostatic | — | — | — | 1 (25.0) | — | — | — | — | 1 (8.3) |
| hypotension | | | | | | | | | |

Abbreviations: PT = Preferred Term; SAD = single-ascending dose; SOC = System Organ Class; TEAE = treatment-emergent adverse event.
Split dosing was the total dose divided evenly into 2 aliquots given 4 hours apart.
TEAEs occurred after the start of double-blind treatment.

One patient reached PD protocol stopping criteria for individual dose escalation during the third period. The stopping criterion at the time was an increase in SET of at least 50 ms on two sequential echocardiograms (later changed to 75 ms on two sequential echocardiograms or 110 ms on any single echocardiogram). After receiving 350 mg of Compound I, SET in one patient was prolonged by ˜63 ms at 1.5 and 3 h postdose and then was prolonged <35 ms at 6 and 9 hours postdose. There were no clinical or ECG findings and no increase in troponin levels. There was no further dosing of this patient. Mean SET prolongation for all patients during 3 to 9 hours postdose at 350 mg was 16.2 ms.

One 67 year-old male subject with HFrEF with a long-standing history (20 years) of ischemic heart disease underwent 4 treatment periods: the first 3 periods were 175 mg, 350 mg, and placebo in that sequence with doses separated by 14 days. Mild dyspnea and fatigue were noted while receiving 175 mg and placebo. Twenty-eight days after the third period, the subject started the fourth period and received 550 mg. Approximately 12 to 24 hours after dosing, the subject complained of moderate dyspnea and cardiac discomfort. There were no new ECG changes suggestive of ischemia. The subject's plasma concentrations of Compound I during the episode ranged from 3400 to 4900 ng/mL. The subject also experienced an AE of troponin increase from normal value pre-dose to a maximum troponin I level of 0.12 ng/mL (4×ULN for the assay) at 24 hours post-dose. Troponin I level began to descend by 36 hours after dosing and was normal by the time of the follow-up visit 7 days after the last dose. These TEAEs resolved without intervention and were considered possibly related to study drug. The SRC reviewed this event and considered it a possible myocardial injury.

Across the 2 SAD cohorts in HFrEF patients, a total of 12 subjects were exposed to 12 placebo periods and 30 active-treatment periods. A transient troponin increase was observed in 3 subjects (3/12=25%) in a total of 3 active-treatment periods (out of a total of 30 active treatment periods) at doses ranging from 175 to 550 mg (3/30=10%) versus none (0/8) during placebo periods. With the exception of the case described above, all other troponin increases were asymptomatic. There was no observed instance of troponin increase that was associated with ECG changes suggestive of ischemia. All instances of troponin elevation were transient and resolved without sequelae.

An analysis of the study ECGs for all patients showed no signal for QTcF increase. An assessment of the Holter monitoring for all patients revealed no signal for increased total atrial ectopy, atrial fibrillation, ventricular ectopy, or NSVT runs with Compound I, as compared to placebo.

The PK and PD data from the patients treated with Compound I in this study provide preliminary evidence of the expected positive inotropic effects of Compound I in patients with HFrEF, which are associated with modest increase in SET and no discernable impact on relaxation. The changes in PD parameters are in a range that could translate into clinical benefit during chronic therapy.

PK/PD and Safety Data from Part 2 (MAD)

A total of 40 subjects across 4 cohorts received 7 days of treatment with placebo or Compound I at doses of 50 mg (with food), 75 mg (1 cohort with food, 1 cohort with 4 hours fasting), or 100 mg (with food), BID (see FIG. 5B and Table 14).

TABLE 14
MAD Cohort Dosing

| | | Number of patients treated |
|---|---|---|
| Cohort | Dose | with Compound I* |
| A (n = 8) | 75 mg BID (4 h fasting) | 6 |
| B (n = 12) | 50 mg BID (with food) | 9 |
| C (n = 12) | 75 mg BID (with food) | 9 |
| D (n = 8) | 100 mg BID (with food) | 6 |

*1:3 placebo to active randomization ratio. Cohort A (fasting 2 h before and 2 h after dose); Cohorts B, C, D (dose taken with food). BID, twice daily.

An analysis of PK, PD, clinical safety, and tolerability data is shown below.

To account for the fact that HFrEF subjects may have elevated troponin values related to their background HFrEF condition (i.e., not related to ischemia or infarction), and that troponin values may fluctuate around the upper limit of normal (ULN), a “troponin increase” in the study was defined as follows:
If troponin was within normal ranges pre-dose (≤0.03 ng/mL for troponin I and <0.014 ng/ml for hs-troponin T), subject was identified as having a “troponin increase” if subject experienced at least 1 value during or post-end of treatment >2×ULN (>0.06 for troponin I or ≥0.028 for hs-troponin T).
If troponin was above ULN pre-dose, a subject was identified as having “troponin increase” if subject experienced at least 1 value during or post-treatment that was increased by >0.03 ng/mL as compared to Baseline (for troponin I or hs-troponin T).

### Cohort A

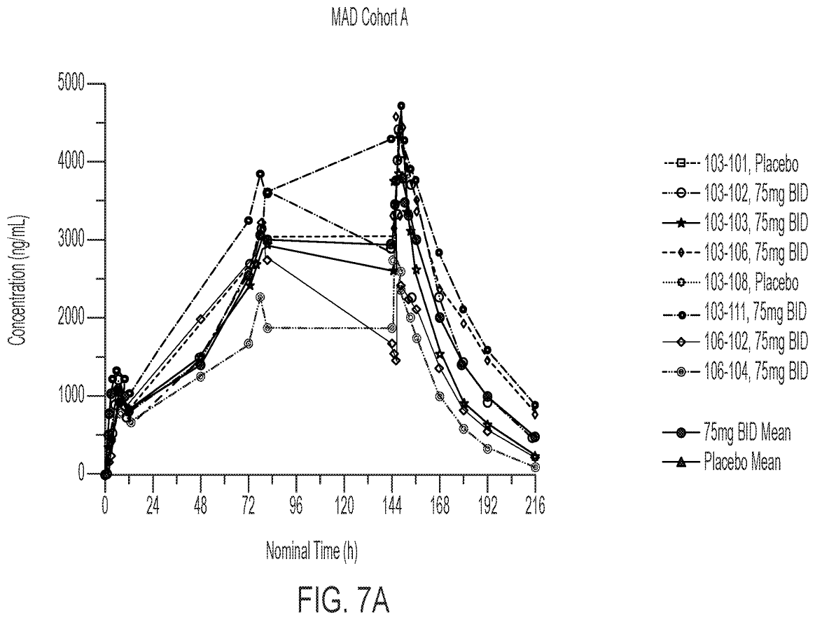

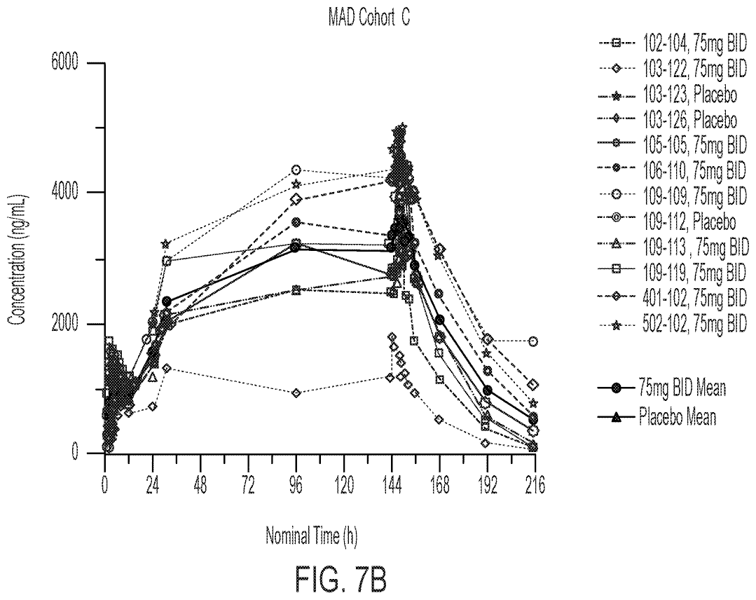

Eight patients with stable heart failure were enrolled and randomized to receive Compound I (six patients) or placebo (two patients) at an oral dose of 75 mg twice daily for six days and a single dose on the seventh day, with fasting two hours before and two hours after dosing. Pharmacokinetic parameter results are summarized in Table 15 below. As shown in FIG. 7, Panel A, steady-state plasma concentrations were reached at approximately 3 days or 72 hours after the first dose.

### Cohort B

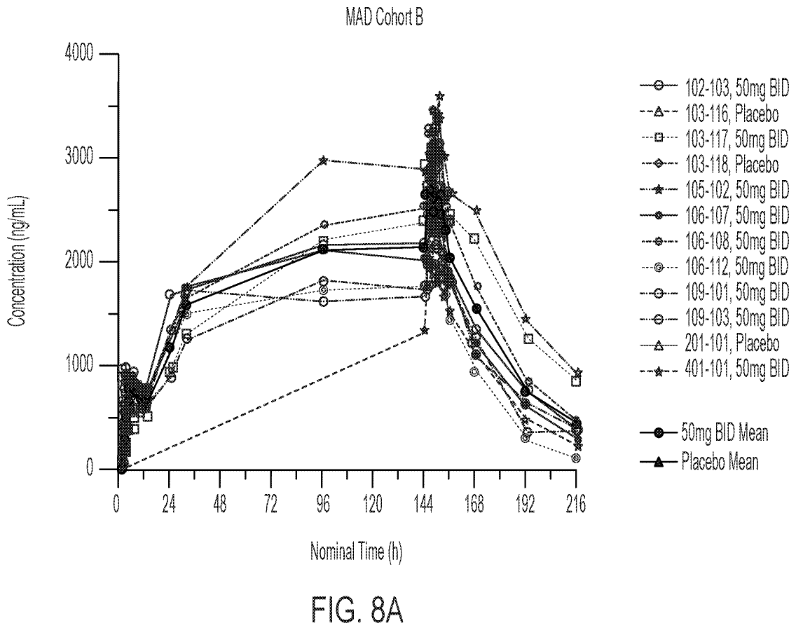

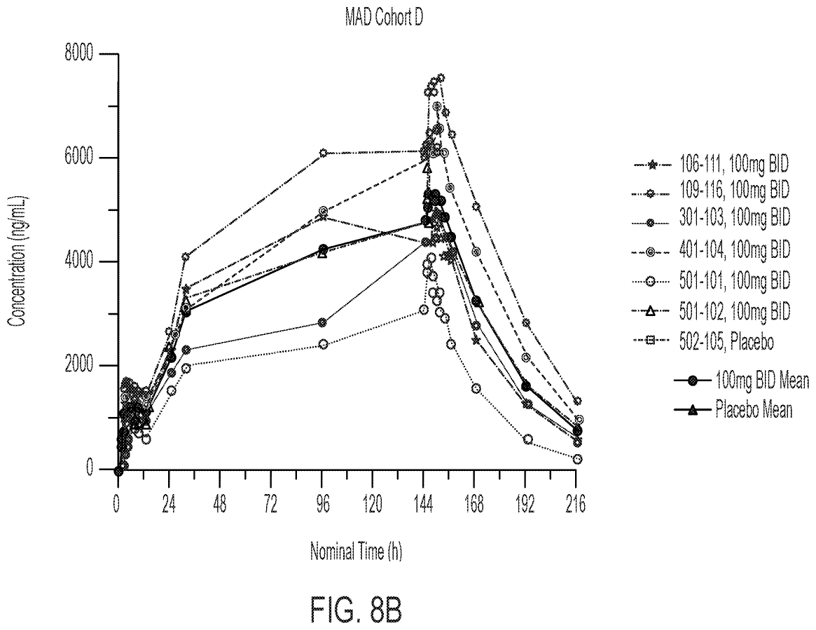

Twelve patients with stable heart failure were enrolled and randomized to receive Compound I (nine patients) or placebo (three) at an oral dose of 50 mg with food twice daily for six days and a single dose on the seventh day. Pharmacokinetic parameter results are summarized in Table 15 below. As shown in FIG. 8, Panel A, steady-state plasma concentrations for these patients were reached at approximately 4 days or 96 hours after the first dose.

### Cohort C

Twelve patients with stable heart failure were enrolled and randomized to receive Compound I (nine patients) or placebo (three) at an oral dose of 75 mg with food twice daily for six days and a single dose on the seventh day. Pharmacokinetic parameter results are summarized in Table 15 below. Plasma concentrations over time are shown in FIG. 7, Panel B.

### Cohort D

Eight patients with stable heart failure were enrolled and randomized to receive Compound I (six patients) or placebo (two patients) at an oral dose of 100 mg twice daily for six days and a single dose on the seventh day, with fasting two hours before and two hours after dosing. Pharmacokinetic parameter results are summarized in Table 15 below. Plasma concentrations over time are shown in FIG. 8, Panel B.

Table 15 summarizes the PK parameters calculated from data obtained from MAD cohorts A-D. Overall, t_{1/2} was consistent with data acquired in SAD cohorts. Cₘₐₓ, Tₘₐₓ, and AUCₜₐᵤ were consistent with modeled parameters.

TABLE 15
Summary of Individual and Mean Pharmacokinetic Parameters for Patient Subjects in MAD Cohorts A-D

| | | | | | | | | | Accumulation | | | |
|---|---|---|---|---|---|---|---|---|---|---|---|---|
| | | Cmax, | AUCtau, | Cmin, | Cmax, | Cavg, | | | Ratio | | | |
| Cohort | Subject | Day 1 | Day 1 | SS | SS | ss | AUC_TAU | Cmax/ | | AUC | t_{1/2, λz} | Accumulation_{—} |
| (Dose) | ID | (ng/mL) | (hr*ng/mL) | (ng/mL) | (ng/mL) | (ng/mL) | (hr*ng/mL) | Cmin | Cmax | tau | (hr) | Index |
| MAD | 103-102 | 1100 | 10610 | 2880 | 4470 | 3774 | 45290 | 1.55 | 4.06 | 4.27 | 20.55 | 3.00 |
| Cohort | 103-103 | 1250 | 11570 | 2610 | 3740 | 3188 | 38250 | 1.43 | 2.99 | 3.31 | 17.21 | 2.61 |
| A | 103-106 | 1060 | 10560 | 3050 | 4580 | 3846 | 46160 | 1.50 | 4.32 | 4.37 | 27.41 | 3.82 |
| (75 mg | 103-111 | 1360 | 12360 | 3510 | 4710 | 4079 | 48950 | 1.34 | 3.46 | 3.96 | 28.02 | 3.89 |
| BID) | 106-102 | 958 | 8025 | 1450* | 2400* | 2108* | 25290* | 1.66* | 2.51* | 3.15* | 18.78* | 2.79* |
| | 106-104 | 981 | 9501 | 1740 | 2750 | 2215 | 26580 | 1.58 | 2.80 | 2.80 | 13.84 | 2.21 |
| | Mean | 1118 | 10440 | 2758 | 4050 | 3420 | 41046 | 1.482 | 3.53 | 3.74 | 21.4 | 3.11 |
| | SD | 157.5 | 1530 | 656.5 | 818.4 | 749.5 | 8996 | 0.096 | 0.658 | 0.671 | 6.23 | 0.739 |
| | CV % | 14.09 | 14.66 | 23.8 | 20.2 | 21.9 | 21.9 | 6.49 | 18.6 | 17.9 | 29.1 | 23.8 |
| MAD | 102-103 | 933 | 7428 | 1900 | 2950 | 2478 | 29740 | 1.55 | 3.16 | 4.00 | 25.81 | 3.63 |
| Cohort | 103-117 | 548 | 5050 | 2380 | 2920 | 2681 | 32170 | 1.23 | 5.33 | 6.37 | 37.48 | 5.03 |
| B | 105-102 | 674 | 6754 | 2640 | 3220 | 2940 | 35280 | 1.22 | 4.78 | 5.22 | 32.77 | 4.46 |
| (50 mg | 106-107 | 830 | 8138 | 1810 | 2560 | 2213 | 26560 | 1.41 | 3.08 | 3.26 | 19.93 | 2.93 |
| BID) | 106-108 | 891 | 8855 | 2370 | 3580 | 3002 | 36030 | 1.51 | 4.02 | 4.07 | 24.77 | 3.51 |
| | 106-112 | 901 | 7249 | 1440 | 2380 | 1991 | 23890 | 1.65 | 2.64 | 3.30 | 15.57 | 2.42 |
| | 109-101 | 696 | 6347 | 1720 | 2120 | 1919 | 23030 | 1.23 | 3.05 | 3.63 | 22.23 | 3.20 |
| | 109-103 | 974 | 8620 | 1650 | 2440 | 2070 | 24840 | 1.48 | 2.51 | 2.88 | 22.21 | 3.20 |
| | 401-101 | NA | NA | 1320* | 2060* | 1725* | 20700* | 1.56* | NA | NA | 30.87* | 4.23* |
| | Mean | 805.9 | 7305 | 1989 | 2771 | 2336 | 28943 | 1.41 | 3.57 | 4.09 | 25.1 | 3.55 |
| | SD | 149.9 | 1262 | 422.7 | 484.2 | 461 | 5133.1 | 0.167 | 1.03 | 1.16 | 7.04 | 0.839 |
| | CV % | 18.6 | 17.3 | 21.3 | 17.5 | 19.7 | 17.7 | 11.8 | 28.8 | 28.4 | 28.0 | 23.6 |
| MAD | 102-104 | 1780 | 13270 | 1740 | 3860 | 2695 | 32340 | 2.22 | 2.17 | 2.44 | 13.55 | 2.18 |
| Cohort | 103-122 | 1020 | 8280 | 954 | 1840 | 1308 | 15700 | 1.93 | 1.80 | 1.90 | 15.34 | 2.39 |
| C | 105-105 | 1190 | 10790 | 2660 | 3810 | 3126 | 37520 | 1.43 | 3.20 | 3.48 | 20.46 | 2.99 |
| (75 mg | 106-110 | 1020 | 9731 | 3240 | 4140 | 3738 | 44850 | 1.28 | 4.06 | 4.61 | 23.11 | 3.31 |
| BID) | 109-109 | 1030 | 6976 | 3910 | 4940 | 4432 | 53180 | 1.26 | 4.80 | 7.62 | 41.54 | 5.51 |
| | 109-113 | 1150 | 9166 | 2530 | 3220 | 2963 | 35560 | 1.27 | 2.80 | 3.88 | 15.67 | 2.43 |
| | 109-119 | 1650 | 14840 | 2440 | 3930 | 3206 | 38470 | 1.61 | 2.38 | 2.59 | 13.72 | 2.20 |
| | 401-102 | 1020 | 10560 | 3930 | 4720 | 4267 | 51200 | 1.20 | 4.63 | 4.85 | 30.37 | 4.17 |
| | 502-102 | 1360 | 11840 | 3900 | 5010 | 4474 | 53690 | 1.28 | 3.68 | 4.53 | 24.06 | 3.42 |
| | Mean | 1247 | 10610 | 2812 | 3941 | 3357 | 40280 | 1.50 | 3.28 | 3.99 | 21.98 | 3.18 |
| | SD | 290.2 | 2451 | 1041 | 983.9 | 1015 | 12180 | 0.355 | 1.08 | 1.72 | 9.248 | 1.10 |
| | CV % | 23.3 | 23.1 | 37.0 | 25.0 | 30.2 | 30.2 | 23.7 | 32.9 | 43.1 | 42.1 | 34.6 |
| MAD | 106-111 | 1560 | 11590 | 4090 | 5210 | 4656 | 55880 | 0.2738 | 3.34 | 4.82 | 24.34 | 3.46 |
| Cohort | 109-116 | 1660 | 13660 | 6140 | 7520 | 6920 | 83040 | 0.2248 | 4.53 | 6.08 | 26.96 | 3.77 |
| D | 301-103 | 1730 | 12840 | 3820 | 4880 | 4315 | 51790 | 0.2775 | 2.82 | 4.03 | 20.99 | 3.06 |
| (100 | 401-104 | 1710 | 16580 | 5230 | 6600 | 6058 | 72700 | 0.262 | 3.86 | 4.38 | 23.11 | 3.31 |
| mg | 501-101 | 1040 | 10080 | 2480 | 4000 | 3236 | 38830 | 0.6129 | 3.85 | 3.85 | 17.23 | 2.61 |
| BID) | 501-102 | 1180 | 12660 | 4240 | 5850 | 4995 | 59940 | 0.3797 | 4.96 | 4.73 | 25.69 | 3.62 |
| | Mean | 1480 | 12900 | 4333 | 5677 | 5030 | 60360 | 0.3384 | 3.89 | 4.65 | 23.05 | 3.30 |
| | SD | 295.9 | 2182 | 1252 | 1260 | 1304 | 15650 | 0.1440 | 0.774 | 0.796 | 3.522 | 0.418 |
| | CV % | 20.0 | 16.9 | 28.9 | 22.2 | 25.9 | 25.9 | 42.6 | 19.9 | 17.1 | 15.3 | 12.7 |

Abbreviations: AUCtau, area under the plasma concentration-time curve during dosing interval(Tau); BID, twice daily; Cmax, maximum/peak concentration after dose; Cmin, minimum/trough concentration during dosing interval; CV, coefficient of variation; MAD, multiple ascending doses; SD, standard deviation; SS, steady state; t_{1/2, λz}, terminal elimination half-life.
Accumulation index was estimated based λz and Tau (dosing interval).
*Subject 106-102 in Cohort A missed doses on study Day 6 and Day 7. Data on Day 7 were excluded for statistical analysis. Subject 401-101 in Cohort B missed doses on Days 1-6 and was excluded for mean concentration calculation.

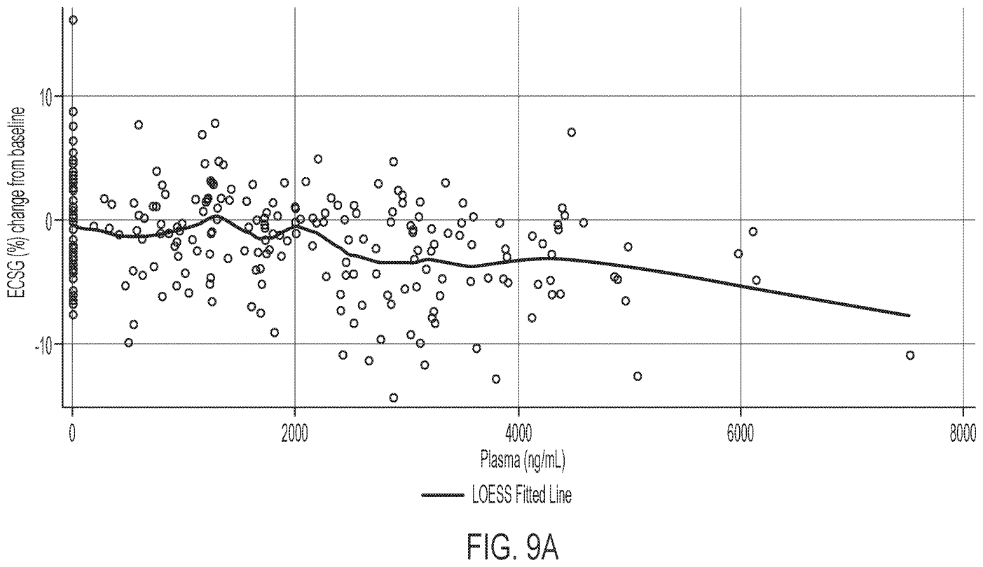

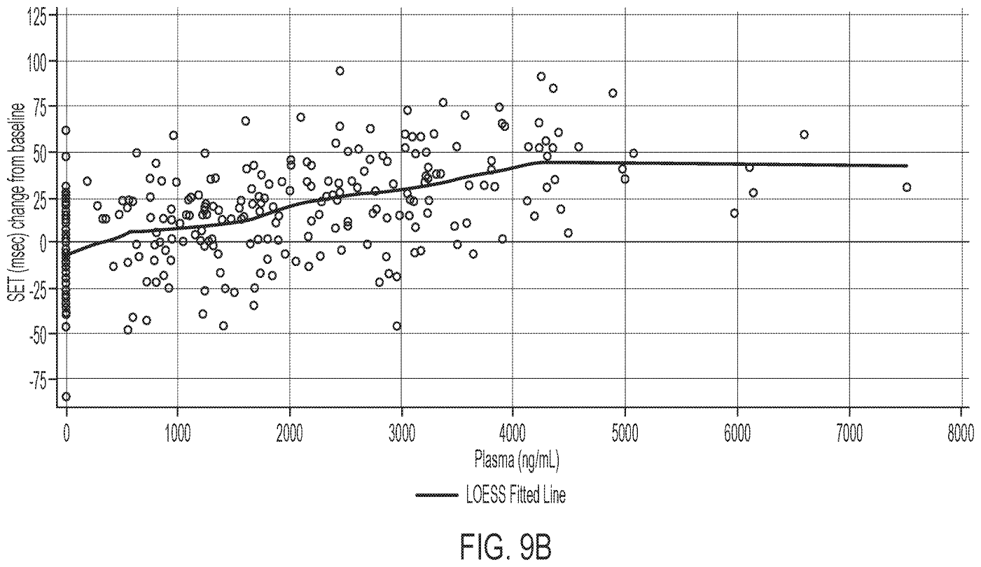

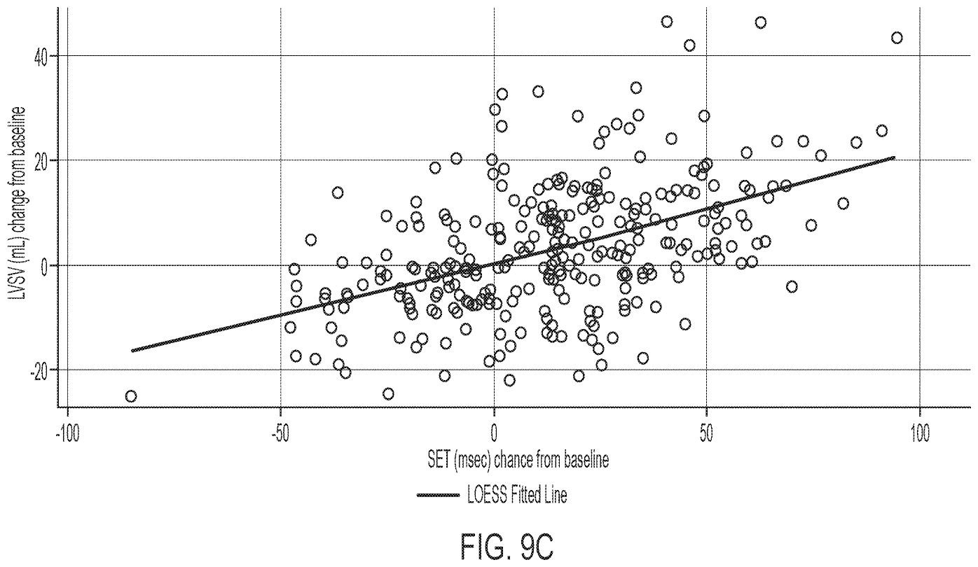

The pharmacodynamic effects of Compound I on echocardiographic markers of cardiac structure and function were analyzed by Compound I plasma concentration groups: <2000 ng/mL (lower concentration group), 2000-3500 ng/ml (medium concentration group) and >3500 ng/ml (higher concentration group) (Table 16) and with PK-PD scatterplots (FIGS. 9A-9C). The medium concentration group corresponds to steady-state plasma concentrations achieved with 50 mg BID (Table 17). A total of 526 echocardiograms were performed from which the PK-PD analysis was derived.

TABLE 16
MAD Cohorts - Change from baseline (placebo-corrected) in echocardiography
parameters by Compound I plasma concentration group

| | Mean change (SE)^{b, c} by Compound I | | |
|---|---|---|---|
| | plasma concentrations group | | |
| Baseline^{a} | <2000 ng/mL | 2000-<3500 ng/mL | ≥3500 ng/mL |
| (n = 40) | (n = 30) | (n = 26) | (n = 13) |

Plasma concentration (ng/mL)

| Mean (SD) | — | 1169 | (454) | 2716 | (425) | 4448 | (855) |
|---|---|---|---|---|---|---|---|
| Median (range) | | 1220 | (183-1960) | 2740 | (2000-3490) | 4290 | (3500-7520) |

Main measures of LV systolic function

| | | | | | | | | |
|---|---|---|---|---|---|---|---|---|
| LVSV (mL) | 59 | (13) | 3.1 | (1.8) | 7.8** | (2.0) | 5.7* | (2.5) |
| LVEF (%) | 32 | (6) | −0.3 | (0.9) | 1.1 | (0.9) | 2.3 | (1.2) |
| LVFS (%) | 18 | (5) | 0.5 | (0.5) | 0.8 | (0.6) | 0.5 | (0.7) |
| SET (ms) | 286 | (29) | 15** | (3.5) | 36** | (3.8) | 48** | (4.7) |

Other measures of LV systolic function

| | | | | | | | | |
|---|---|---|---|---|---|---|---|---|
| LVGLS (%) | −11.2 | (2) | −0.3 | (0.3) | −0.9* | (0.4) | −1.0* | (0.4) |
| LVGCS (%) | −14.1 | (4.3) | −0.4 | (0.7) | −2.1** | (0.7) | −3.3** | (0.8) |
| s′ (lateral) | 5.2 | (1.3) | 0.2 | (0.2) | 0.6** | (0.2) | 0.3 | (0.2) |

LV dimensions and volumes

| | | | | | | | | |
|---|---|---|---|---|---|---|---|---|
| LVESD (mm) | 48 | (8) | −0.8 | (0.4) | −1.3** | (0.5) | −1.8** | (0.6) |
| LVEDD (mm) | 58 | (7) | −0.5 | (0.3) | −0.9** | (0.3) | −1.8** | (0.4) |
| LVESVi (mL/m²) | 60 | (22) | −0.9 | (1.3) | −1.3 | (1.4) | −4.6** | (1.7) |
| LVEDVi (mL/m²) | 88 | (27) | −1.1 | (1.5) | −1.1 | 1.6) | −5.2* | (2.0) |

Composite measure of systolic and diastolic function

| | | | | | | | | |
|---|---|---|---|---|---|---|---|---|
| Tei index | 0.66 | (0.2) | −0.05 | (0.03) | −0.08** | (0.03) | −0.02 | (0.03) |

Relaxation/diastolic function

| | | | | | | | | |
|---|---|---|---|---|---|---|---|---|
| e′ (lateral) | 6.3 | (1.9) | −0.2 | (0.2) | 0.1 | (0.2) | −1.0** | (0.3) |
| E/e′ (lateral) | 12.4 | (5.8) | 0.8 | (0.5) | −0.7 | (0.6) | 0.3 | (0.7) |
| E-wave peak (cm/s) | 69 | (25) | −3.8 | (2.1) | −2.1 | (2.2) | −10** | (2.7) |
| A-wave peak (cm/s) | 74 | (25) | 4.1* | (1.9) | 6.1** | (2.1) | 4.3 | (2.6) |
| A-wave | 135 | (25) | 6.0 | (3.1) | 5.9 | (3.3) | 11.9** | (4.0) |
| duration (msec) | | | | | | | | |
| E/A ratio | 1.0 | (0.5) | −0.1** | (0.04) | −0.1** | (0.04) | −0.2** | (0.05) |
| IVRT (msec) | 123 | (24) | 2.7 | (5.1) | 10.5 | (5.4) | 27.8** | (6.3) |

Vital signs (supine)

| | | | | | | | | |
|---|---|---|---|---|---|---|---|---|
| Heart rate (bpm) | 66 | (10) | 0.0 | (1.1) | −2.0 | (1.2) | −1.1 | (1.6) |
| SBP (mmHg) | 117 | (18) | −1.5 | (1.6) | −0.8 | (1.8) | −5.2* | (2.3) |
| DBP (mmHg) | 70 | (10) | −0.9 | (1.0) | −0.2 | (1.2) | −1.4 | (1.5) |

Abbreviations: A, late peak wave velocity from mitral inflow Doppler; bpm, beats per minute; DBP, diastolic blood pressure; e′, peak atrioventricular valve annular velocity in early diastole; E, early peak wave velocity from mitral inflow Doppler; IVRT, isovolumic relaxation time; LS, least-squares; LV, left ventricular; LVEDD, left ventricular end-diastolic diameter; LVEDVi, left ventricular end-diastolic volume index; LVEF, left ventricular ejection fraction; LVESD, left ventricular end systolic diameter; LVESVi, left ventricular end systolic volume index; LVFS, left ventricular fractional shortening; LVGCS, left ventricular global circumferential strain; LVGLS, left ventricular global longitudinal strain; LSVS, left ventricular stroke volume; MR, mitral regurgitation; SBP, systolic blood pressure; SD, standard deviation; SE, standard error; SET, systolic ejection time; TTE, transthoracic echocardiogram.
For the analysis, all assessments are included in the column corresponding to the Compound I concentration reached concomitantly to the assessments. As a result, 4 patients contributed to the lower (<2,000 ng/mL) Compound I concentration group only, 13 patients contributed to both the lower and medium (2,000-<3500 ng/mL) Compound I concentration groups, and 13 patients to all three Compound I concentration groups.
^{a}Absolute arithmetic mean values and SD for the baseline measurement for all Compound I-treated patients, excluding patients receiving placebo.
^{b}LS mean difference (SE) between each plasma concentration group (<2000 ng/mL, 2000-<3500 and ≥3500 ng/mL) and placebo (concentration = 0) in TTE parameters' change from baseline.
^{c}SE of LS mean difference = SE of the LS mean difference.
*p < 0.05.
**p < 0.01.

TABLE 17
Compound I steady-state (Day 9) plasma concentrations

| | | Pre-dose | Post-dose maximum |
|---|---|---|---|
| | Dosing | concentration | concentration |
| Cohort | regimen | (ng/mL)^{a} | (ng/mL)^{a} |
| B (n = 8) | 50 mg BID | 2096 (20.0%) | 2735 (17.5%) |
| A + C (n = 14) | 75 mg BID | 2930 (36.8%) | 3862 (27.7%) |
| D (n = 6) | 100 mg BID | 4694 (25.5%) | 5560 (22.8%) |

* includes all patients who received Compound I treatment as per protocol. BID, twice daily

Treatment with Compound I was associated with a concentration-dependent increase in stroke volume (mean placebo-corrected increase of 7.8 [p<0.01] and 5.7 mL [p<0.05] at the medium and higher concentration groups, respectively). Compound I also improved LV longitudinal as well as circumferential strain (mean placebo-corrected decrease of −2.1 and −3.3% at the medium and higher concentration groups, respectively) and reduced LV dimensions (mean placebo-corrected decrease in LVESD of −1.3 [p<0.01] and −1.8 mm [p<0.01] at the medium and higher concentration groups, respectively). A non-significant increase in LVEF was noted. A dose-dependent increase in SET was observed, with a mean placebo-corrected increase of 36 (p<0.01) and 48 msec (p<0.01) observed at the medium and higher concentration groups, respectively (FIG. 9B). A correlation was seen between the change from baseline in LVSV and the change from baseline in SET (FIG. 9C). No significant changes in relaxation (e′, peak E wave) was observed in the medium concentration group. E/A was decreased due to an increase in A peak wave velocity. In the higher concentration group, a decrease in e′, peak E wave (−10 cm/s, p<0.01) and E/A were observed. No change in filling pressures (E/e′) was noted in the medium or higher concentration group. There was no significant change in vital signs at low and medium concentrations. In the higher concentration group, there was a decrease in systolic blood pressure, and no change in diastolic blood pressure or heart rate. No increase in QTc was observed Holter monitoring revealed no increase in ventricular arrhythmias with Compound I compared with placebo.

Treatment-emergent adverse events (TEAEs) were reported in 17 (57%) Compound I and 4 (40%) placebo patients, with no organ specificity, and no apparent relation to dose (Table 18). All TEAEs observed with Compound I (except one) were considered to be of mild intensity and/or unrelated to study treatment, and all TEAEs resolved without sequelae. One patient had two episodes of non-sustained ventricular tachycardia (NSVT), considered to be of moderate intensity and related to Compound I. The patient also had NSVT on Holter at baseline. No TEAE led to permanent treatment discontinuation or death. One serious AE was reported in the study, hyperkalemia, in a patient who received Compound I. The event resolved and was not considered related to study treatment. The most common TEAEs in patients receiving Compound I (each reported in 2 patients) were: ALT increase (in both patients, events were mild, non-related to study treatment, and self-resolved), contact dermatitis (in both patients, events were mild, non-related to study treatment), fatigue, troponin increase and non-sustained ventricular tachycardia (NSVT episodes observed in 2 patients, in whom NSVTs were also observed on Holter at baseline). A transient and asymptomatic increase in either troponin I or hs-troponin T was seen in 7 (23%) patients treated with Compound I (2/9 patients at 50 mg, 2/15 patients at 75 mg and 3/6 patients at 100 mg; all 7 patients experienced troponin I increase, of whom one patient treated with 100 mg also had hs-troponin T increase) versus none on placebo (Table 19). None of the troponin increases observed in the MAD Cohorts were associated with symptoms or with ECG changes suggestive of ischemia.

TABLE 18
Treatment-Emergent Adverse Events (TEAEs) in MAD Cohorts

| | | Compound I | | | |
|---|---|---|---|---|---|
| | | | Cohort | | Total |
| | Total | Cohort B | A + C | Cohort D | Compound |
| | placebo | 50 mg BID | 75 mg BID | 100 mg BID | I |
| Adverse Events | (n = 10) | (n = 9) | (n = 15) | (n = 6) | (n = 30) |

Number of patients (%) with AEs

| | | | | | | | | | | |
|---|---|---|---|---|---|---|---|---|---|---|
| Any TEAE | 4 | (40.0) | 7 | (77.8) | 6 | (40.0) | 4 | (66.7) | 17 | (56.7) |

| | | | | | | | |
|---|---|---|---|---|---|---|---|
| Any serious TEAE | 0 | 0 | 1 | (6.7) | 0 | 1 | (3.3) |

| | | | | | |
|---|---|---|---|---|---|
| Any TEAE leading to | 0 | 0 | 0 | 0 | 0 |
| permanent treatment | | | | | |
| discontinuation | | | | | |
| Any AE leading to death | 0 | 0 | 0 | 0 | 0 |

Occurred in ≥10.0% of patients in any group, n (%)

| | | | | | | | | |
|---|---|---|---|---|---|---|---|---|
| Alanine aminotransferase | 0 | 1 | (11.1) | 1 | (6.7) | 0 | 2 | (6.7) |
| increased | | | | | | | | |

| | | | | | | | |
|---|---|---|---|---|---|---|---|
| Dermatitis contact | 0 | 2 | (22.2) | 0 | 0 | 2 | (6.7) |

| Fatigue | 0 | 0 | 2 | (13.3) | 0 | | 2 | (6.7) |
|---|---|---|---|---|---|---|---|---|
| Troponin increased | 0 | 0 | 1 | (6.7) | 1 | (16.7) | 2 | (6.7) |

| | | | | | | | | |
|---|---|---|---|---|---|---|---|---|
| Ventricular tachycardia | 0 | 1 | (11.1) | 0 | 1 | (16.7) | 2 | (6.7) |

| | | | | | | | | |
|---|---|---|---|---|---|---|---|---|
| Anemia | 1 | (10) | 0 | 1 | (6.7) | 0 | 1 | (3.3) |

| | | | | | | | |
|---|---|---|---|---|---|---|---|
| Abdominal discomfort | 0 | 1 | (11.1) | 0 | 0 | 1 | (3.3) |
| Application site erosion | 0 | 1 | (11.1) | 0 | 0 | 1 | (3.3) |

| | | | | | | | |
|---|---|---|---|---|---|---|---|
| Arthropod bite | 0 | 0 | 0 | 1 | (16.7) | 1 | (3.3) |
| Blood creatinine increased | 0 | 0 | 0 | 1 | (16.7) | 1 | (3.3) |

| | | | | | | | |
|---|---|---|---|---|---|---|---|
| Blood creatine | 0 | 1 | (11.1) | 0 | 0 | 1 | (3.3) |
| phosphokinase increased | | | | | | | |

| | | | | | | | | |
|---|---|---|---|---|---|---|---|---|
| Cough | 1 | (10) | 0 | 1 | (6.7) | 0 | 1 | (3.3) |

| | | | | | | | |
|---|---|---|---|---|---|---|---|
| Fluid overload | 0 | 1 | (11.1) | 0 | 0 | 1 | (3.3) |

| | | | | | | | |
|---|---|---|---|---|---|---|---|
| Gingival pain | 0 | 0 | 0 | 1 | (16.7) | 1 | (3.3) |

| | | | | | | | |
|---|---|---|---|---|---|---|---|
| Hyperkalemia | 0 | 1 | (11.1) | 0 | 0 | 1 | (3.3) |
| Infusion site erythema | 0 | 1 | (11.1) | 0 | 0 | 1 | (3.3) |
| Rash | 0 | 1 | (11.1) | 0 | 0 | 1 | (3.3) |

| | | | | | | |
|---|---|---|---|---|---|---|
| Arthralgia | 1 | (10) | 0 | 0 | 0 | 0 |
| Back pain | 1 | (10) | 0 | 0 | 0 | 0 |
| Dry eye | 1 | (10) | 0 | 0 | 0 | 0 |
| Nasopharyngitis | 1 | (10) | 0 | 0 | 0 | 0 |
| Renal failure | 1 | (10) | 0 | 0 | 0 | 0 |
| Renal impairment | 1 | (10) | 0 | 0 | 0 | 0 |
| Testicular pain | 1 | (10) | 0 | 0 | 0 | 0 |

AE, adverse event; BID, twice daily; TEAE, treatment-emergent adverse event.

TABLE 19
Serum Troponin Concentrations in MAD Cohorts

| | Placebo | | Total Compound I | |
|---|---|---|---|---|
| Troponin I (ng/mL, ULN = 0.03) | (n = 10) | | (n = 30) | |
| Median baseline | 0.010 | | 0.010 | |
| Median change from | 0.005 | (0.03) | 0.010 | (0.87) |
| baseline (max change) | | | | |
| Median peak troponin | 0.020 | (0.05) | 0.025 | (0.88) |
| post dose (max peak) | | | | |
| hs-troponin T^{a} (ng/mL, | (n = 7) | | (n = 22) | |
| ULN = 0.014) | | | | |
| Median baseline | 0.023 | | 0.015 | |
| Median change from | 0.002 | (0.005) | 0.005 | (0.041) |
| baseline (max change) | | | | |
| Median peak troponin | 0.025 | (0.032) | 0.020 | (0.052) |
| post dose (max peak) | | | | |

hs, high-sensitivity; ULN, upper limit of normal.
^{a}hs-troponin T assessment added after study had started.

### SAD and MAD Cohorts: Pharmacokinetic-Pharmacodynamic Relationships

Changes for the main echocardiographic PD parameters from SAD cohorts and MAD cohorts by concentration group are shown in Table 12 and Table 16, respectively. An exposure-related increase in forward flow (˜8 to 9 mL increase in SV) and LV contractility (LV strain) was observed. Myocardial performance (or Tei index, an indicator of combined systolic and diastolic function (Bruch et al., Eur Heart J. (2000)21:1888-95) was improved by approximately 10% in the concentrations ≥2000 ng/mL. SET was moderately increased (<50 msec).

Safety/Tolerability Conclusions from Single and Multiple-Ascending Dose Cohorts

Single-dose (up to 550 mg) and multiple-dose administration (50 to 100 mg BID administered for 7 days) of Compound I in HFrEF subjects was safe and generally well-tolerated. No ischemic changes were observed by ECG and no clinically significant worsening of any arrhythmia was noted. Mild transient troponin increase was occasionally observed with Compound I. In one subject in SAD Cohort 1 receiving a higher dose (550 mg), troponin increase observation was deemed possibly related to myocardial injury (presence of associated symptoms, no ECG changes). In the MAD cohorts, observed mild troponin increase was not associated with symptoms or ECG changes. Mild troponin elevation was also observed with omecamtiv mecarbil, another drug in this class of cardiac myosin activators currently being investigated in a large Phase 3 cardiovascular outcome trial in HFrEF (Teerlink et al., Lancet (2016)388(10062):2895-903); Teerlink et al., JACC Heart Fail. (2020) doi: 10.1016/j.jchf.2019.12.001).

### Example 4: Investigation of Nonlinear Pharmacokinetics of Compound I by Physiologically-Based Pharmacokinetic Modeling

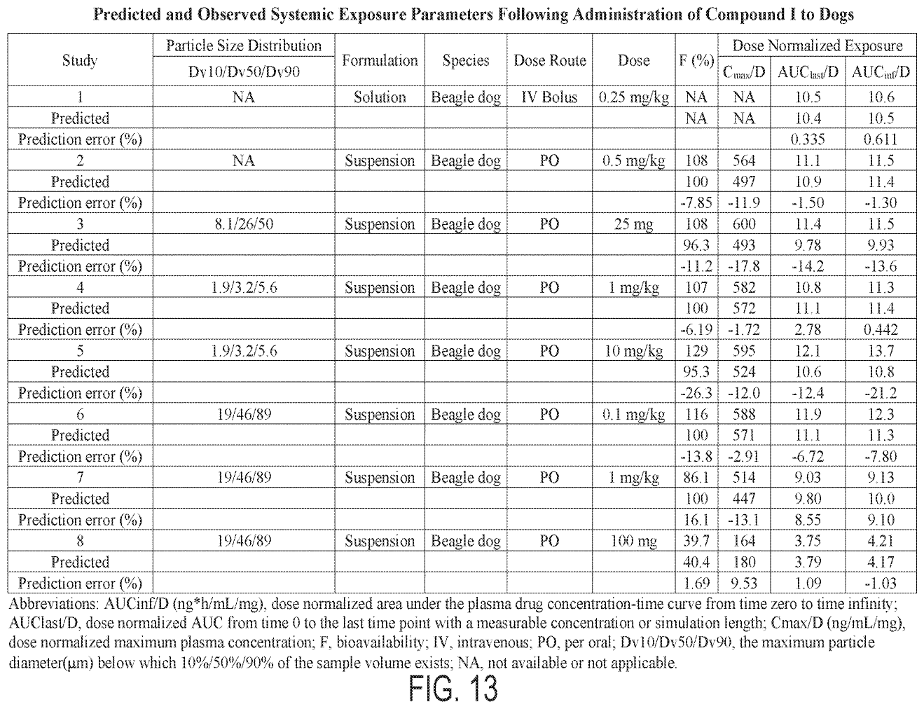

The pharmacokinetics of Compound I have been evaluated in multiple dog studies. As shown in FIG. 13, following oral administration of single doses of Compound I to beagle dogs, systemic exposure of Compound I increased with increasing dose in a less than dose-proportional manner at doses higher than 3 mg/kg. At a single dose <3 mg/kg, the observed oral bioavailability was approximately 100%. This nonlinear pharmacokinetics of Compound I was also observed in humans. As described in Example 1, after oral administration of single ascending doses of 3 to 525 mg to healthy volunteers, systemic exposure (Cₘₐₓ and AUC) increased in a slightly less than dose-proportional manner at doses up to 350 mg, whereas the exposure profile after oral administration of the 525 mg dose was similar to the 350 mg dose. In order to delineate the underlying mechanism responsible for the nonlinear pharmacokinetics, physiologically-based absorption models of Compound I for beagle dogs and healthy volunteers were developed and used to assess the effect of particle size on the in vivo dissolution, absorption, bioavailability, and systemic exposure of Compound I.

### Materials and Methods

### Data Collection

Data used for Compound I physiologically-based pharmacokinetic (PBPK) model development and verification were obtained from in vivo nonclinical studies in dogs (FIG. 13), a clinical study in healthy volunteers (Example 1), and in vitro experiments (Table 20).

### PBPK Model Development

A PBPK mechanistic absorption model was developed by integrating (1) physicochemical and biopharmaceutical properties obtained from in vitro experimental measurements or in silico estimates based on chemical structure using ADMET Predictor (version 7.2) in GastroPlus (Version 9.6); (2) formulation properties of the drug product such as drug substance particle size distribution, formulation type, and rate of release or dissolution; (3) compartmental model kinetic parameters such as systemic clearance, volume of distribution, and inter-compartmental rate constants; and (4) gut physiology parameters such as gastro-intestinal (GI) transit time, pH, absorptive surface area, compartment dimensions and fluid content. The preexisting physiological parameters in GastroPlus (Version 9.6) for American healthy volunteers and beagle dogs under fasted conditions were used without modification.

Particle size distribution data for batches tested were given in FIG. 13. The model input parameters are summarized in Table 20.

A Johnson dissolution model was selected to predict in vivo dissolution rate, which is described by Equation 1 below, including a time-dependent diffusion layer thickness and shape factor to account for changing particle radius during dissolution as well as for dissolution of cylindrical particles.

$$\frac{{dM}_{D}}{dt} = \frac{{D}_{eff}}{ρ {hr}_{t}} \frac{( 1 + 2 s )}{s} ( {C}_{s} - C ) {M}_{s , t} \qquad ( 1 )$$

where M_{D} is dissolved amount, Mᵤ is undissolved amount (at time 0 or t), Cₛ is solubility, Cis concentration of dissolved drug in medium or gut lumen, D_{eff} is diffusion coefficient, p is drug density, rₜ is current particle radius, h is diffusion layer thickness, and s is shape factor defined as length/diameter (s=1 for spherical particles).

### Evaluation of Particle Size Effect

The PBPK model for humans was used to predict in vivo dissolution, absorption, and plasma concentration-time profiles after oral dosing. Simulations were performed using the IR: Suspension dosage form option in GastroPlus with in vitro measured particle size distribution data. The effects of particle size distribution and dose amount on the in vivo dissolution, absorption, bioavailability, and systemic exposure of Compound I were evaluated by parameter sensitivity analysis.

### Results

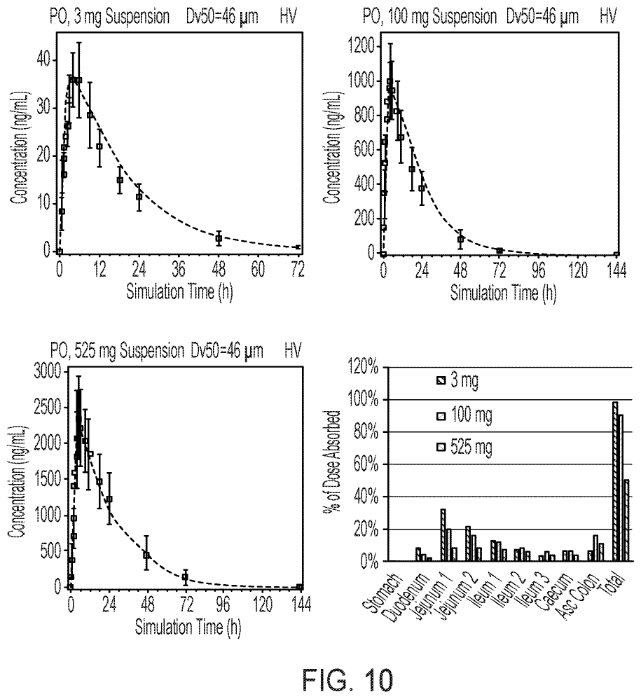

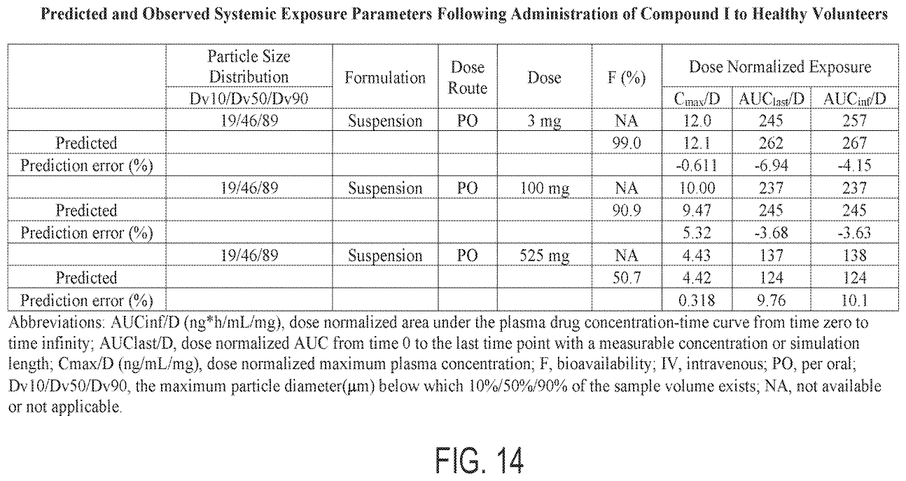

As shown in FIG. 13, the bioavailability of Compound I in beagle dogs was approximately 100% after oral administration of a single dose of Compound I at 25 mg (3 mg/kg) or lower regardless of drug substance particle size distribution. The bioavailability was approximately 40% after oral administration of 100 mg of Compound I with Dv50-46 μm, and more than 100% after oral administration of a 10 mg/kg dose of micronized Compound I (Dv50-3.2 μm). The predicted plasma concentration-time profiles, bioavailability, and systemic exposure parameters (F, Cₘₐₓ, AUCₗₐₛₜ, and AUC_{inf}) were comparable to those observed in various dog studies (FIG. 13) following intravenous or oral administration of single doses of Compound I in solution or suspension formulation under fasted conditions. In humans, the predicted plasma concentration-time profiles (FIG. 10) and systemic exposure parameters (Cₘₐₓ, AUCₗₐₛₜ, and AUC_{inf}) were comparable to those observed in the clinical study described in Example 1 (FIG. 14). The prediction errors for all variables were within-26.3% to 16.1%, which verified both dog and human PBPK models.

TABLE 20
PBPK Model Input Parameters

| Parameter | Values | Sources |
|---|---|---|
| MW (g/mole) | 435.42 | |
| logP | 0.61 | Predicted |
| logD (pH 7.4) | 3.07 | Measured |
| Solubility | ~0.043 mg/mL | Measured |
| (37° C.) | across pH 1.2-9.6 | |
| pKa | pKa₁ = 13.32, pKa₂ = −1.16, | Predicted |
| | pKa₃ = −4.04, pKa₄ = −4.10 | (ACD Lab v12) |
| fᵤₚ (Human) | 14.4% to 16.4% (0.1 to 10 μM) | Measured |
| fᵤₚ (Dog) | 41.6% | Measured |
| B:P (Human) | 0.85 | In vitro measured |
| B:P (Dog) | 0.80 | In vitro measured |
| Permeability | Caco-2: 17.9 (×10⁻⁶ cm/s) | In vitro measured |
| | P_{eff}: 4.369 (×10⁻⁴ cm/s) | Predicted |
| Compartmental | 1-compartmental model | Compartmental model |
| PK Model | V_{d} (L/kg): 0.9648 | analysis of Compound |
| parameters | (Human), 1.51 (Dog) | I in vivo data in |
| | CL (L/h/kg): 0.05694 | human and dog |
| | (Human), 0.0949 (Dog) | |

Abbreviations: B:P, ratio of concentration of drug in blood to plasma; CL, clearance; fup, unbound fraction in plasma; Log D, the logarithm of the distribution coefficient; Log P, the logarithm of the partition coefficient; Peff, the effective permeability; pKa, the negative base-10 logarithm of the acid dissociation constant; Vd, volume of distribution.

The model predicted that bioavailability (F) and fraction of absorption (Fa) in dog and human decreased with increasing doses, consistent with the observed results in dog, suggesting that the reduced dose normalized systemic exposure after oral administration of a batch suspension of Compound I with Dv50=46 μm was caused by the decreased Fa. The reduced bioavailability at higher doses resulted from incomplete absorption due to poor solubility, slow dissolution, and consequent fecal excretion of undissolved drug molecules.

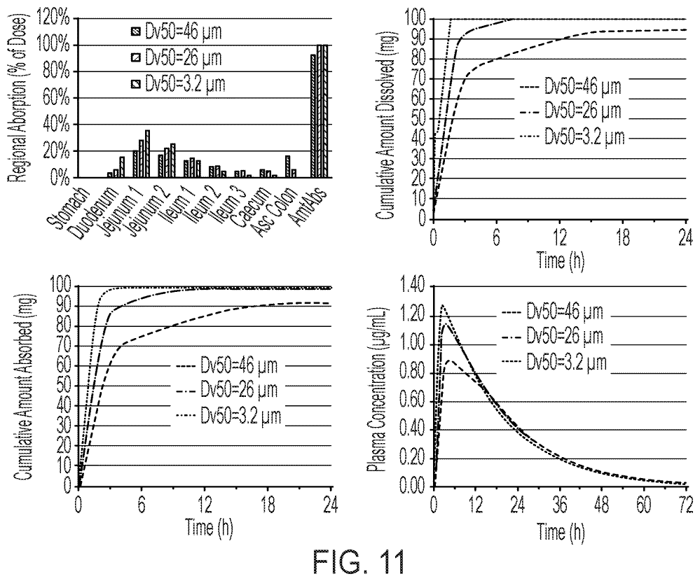

By incorporating in vitro measured particle size distribution information into the GastroPlus model, in vivo dissolution, absorption, and plasma concentration-time profiles were simulated for Compound I with Dv50-46, 26, and 3.2 μm. The simulated in vivo absorption, in vivo dissolution, and plasma concentration-time profiles are depicted in FIG. 11. As shown in FIG. 11, the in vivo dissolution rate was fastest from Compound I with Dv50=3.2 μm, which resulted in the fastest absorption and highest peak plasma concentration. Regional absorption profiles were also different. The percentage of dose absorbed in different segments of the GI tract were different among the three batches as well. The percent of dose absorbed was 97.4% in the small intestine and 2.4% in colon for Compound I with Dv50-3.2 μm, whereas for Compound I with Dv50=46 μm, only 68% of the dose was absorbed in the small intestine but 23.8% of the dose was absorbed in the colon.

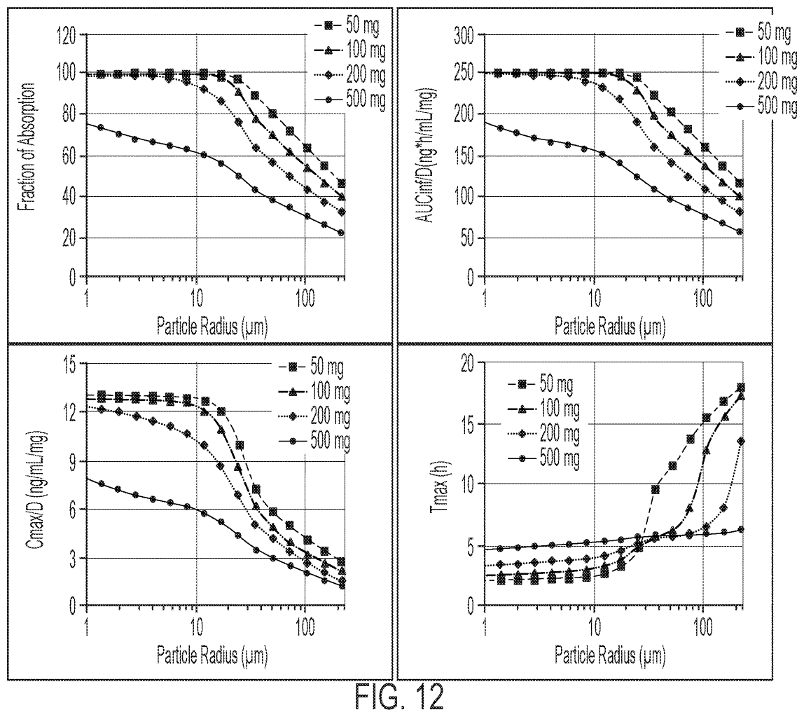

Parameter sensitivity analysis (PSA, FIG. 12) revealed that particle size distribution and dose amount had a significant effect on the in vivo dissolution, absorption, and systemic exposure. At a 500 mg dose, the fraction of absorption and systemic exposure were significantly reduced, even with the micronized drug substance.

The PSA results suggest that the therapeutic dose may be 50 to 100 mg twice daily with optimal absorption when the mean particle diameter is not more than 10 μm.

### Conclusion

The physiologically-based mechanistic absorption models of Compound I for dog and healthy volunteers were developed and verified by reproducing the plasma concentration-time profiles observed in various in vivo studies.

PBPK modeling and simulation demonstrated that absorption of Compound I in both dog and human is dependent on the dose amount and particle size of drug substance. Micronization of the Compound I drug substance can increase the in vivo dissolution rate, and consequently absorption, bioavailability, and systemic exposure at doses higher than 3 mg/kg.

### Alternative Dosing

Plasma concentration profiles with nine different dose regimens (with food intake) were simulated for a targeted steady state mean concentration of 2000 ng/mL to 4000 ng/ml (except 25 mg BID group for special population, ˜ 1000 ng/mL). The steady state could be achieved with a loading dose at 2-fold of the maintenance dose for BID dosing regimen and 1.5-fold for QD dosing. See also Table 21 below.

TABLE 21
Exemplary Dosing Regimens

| | Dosing | Loading | | Maintenance | | Total Dose on |
|---|---|---|---|---|---|---|
| Scenario | Frequency | Dose | Dosing Day | Dose | Time to Start | Day 1 |
| 1 | BID | 50 mg | Day 1, AM | 25 mg | Day 1, PM | 75 mg |
| 2 | BID | 75 mg | Day 1, AM | 25 mg | Day 2, AM | 100 mg |
| 3 | BID | 100 mg | Day 1, AM | 50 mg | Day 1, PM | 150 mg |
| 4 | BID | 150 mg | Day 1, AM | 75 mg | Day 1, PM | 225 mg |
| 5 | BID | 125 mg | Day 1, AM | 75 mg | Day 1, PM | 200 mg |
| 6 | QD | 150 mg | Day 1 | 75 mg | Day 2 | 150 mg |
| 7 | QD | 150 mg | Day 1 | 100 mg | Day 2 | 150 mg |
| 8 | QD | 200 mg | Day 1 | 100 mg | Day 2 | 200 mg |
| 9 | QD | 200 mg | Day 1 | 125 mg | Day 2 | 200 mg |

### Example 5: Open-Label Exploratory Study of Oral Compound I in Stable Ambulatory Patients with Primary Dilated Cardiomyopathy Due to MYH7 Mutation

This example describes a study intended to establish preliminary safety and tolerability of treatment with Compound I in patients with dilated cardiomyopathy caused by a MYH7 mutation resulting in detrimental alterations in actomyosin coupling (MYH7-DCM subjects). The study also is intended (1) to establish preliminary effect, compared with baseline, of treatment with Compound I on cardiac pharmacodynamics (PD), as determined by transthoracic echocardiography (TTE) in MYH7-DCM subjects; and (2) to establish preliminary effect of Compound I on daily activity level in MYH7-DCM subjects.

### Materials and Methods

### Study Design

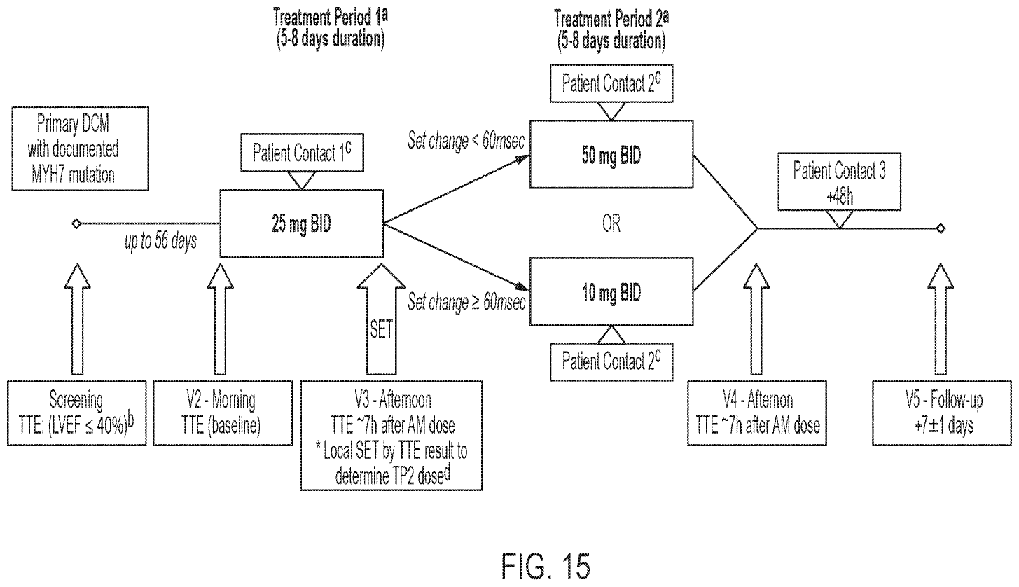

This is a single-cohort, baseline-controlled, sequential two-period, open-label study investigating safety and efficacy of Compound I in stable, ambulatory subjects with primary DCM associated with MYH7 mutation (FIG. 15). Enrollment of up to a total of approximately 12 subjects is planned; however, additional cohorts may be enrolled. The expected study duration ranges from about 4 weeks to 11 weeks, including about 1-8 weeks for screening, 9 to 15 days for IMP dosing and an approximately 1 week (7±1 days) follow-up visit.

### Screening

If allowed by local regulation, subjects may remotely give consent for review of prior genetic testing results to assess preliminary eligibility. Otherwise, anonymized genetic info will be communicated at the time of the first screening visit, after subject has provided his/her informed consent.

Subjects will undergo up to 8 weeks of screening and qualification assessments over one or several study visits, as necessary (Week-8 to Week-1). Screening may be completed over 1 (V1A) to 3 visits (V0, VIA, V1B) and will include but is not limited to: medical history, physical examination, safety laboratory tests, 12-lead ECG (triplicate) and 1 to 2 TTEs.

Abnormal findings from laboratory assessments performed at V1 may be repeated once during screening after corrective treatment (e.g. hemolysis of sample, abnormal potassium levels).

A cardiac rhythm monitoring patch will be placed during the initial TTE if an historical study is being used to qualify the subject. If a second TTE is needed, the patch will be placed at the conclusion of the second TTE/screening visit. Duration of cardiac rhythm monitoring may be between 5 and 14 days. If a patch becomes detached before 5 days, another should be placed.

### Open-Label Treatment Periods

All qualified patients will then undergo 2 open-label treatment periods, with active drug. Both treatment Periods 1 and 2 will each last 5 to 8 days (i.e., Period 1 from D1 through D5-D8 and Period 2 from D5-8 through D9-15), and do not need to have the same duration.

Treatment Period 1 (5-8 days):

Visit 2 (Day 1 of Treatment Period 1) should take place in the morning: Baseline assessments, including a TTE (See Schedule of Assessments, Appendix 1), will be completed prior to administration of the first dose of IMP which is to be taken by the subject prior to leaving the visit. Cardiac rhythm monitoring patch will be placed at the conclusion of Visit 2. Subject will be given IMP supplies to take 25 mg twice daily for up to 8 days.

At the end of the visit, clear instructions shall be provided to subjects on how to take open-label IMP treatment until the next visit (i.e. every day, twice a day, with food at each administration).

Patient Contact 1: One to three days before end of Treatment Period 1 (V3), the subject should be contacted to ensure compliance with study treatment, to remind subject of scheduled time of next visit (Visit 3), and to take treatment (with food) in the morning of Visit 3 about 7 h prior to the scheduled time of the visit.

Visit 3—End of Treatment Period 1 (Day 5 up to Day 8, scheduled in the afternoon); Subjects will return at that visit for an assessment of safety, tolerability, PK and evaluation of PD response.

The scheduling window for Visit 3 is to accommodate weekends and holidays. The last dose of 25 mg IMP will be taken in the morning, approximately 7 hours before this clinic visit. A TTE and other study assessments, including but not limited to laboratory and PK blood samples, 12-lead ECG (triplicate), will be completed. The absence of permanent discontinuation criteria including but not limited to the absence of excessive prolongation of QTcF (>500 msec) will be evaluated. Then, the cardiac sonographer at each local site should carefully measure SET. The SET change from baseline value (i.e. change from SET determined at V2) will determine the dose for Treatment Period 2, either 50 mg BID beginning that evening or 10 mg BID beginning the following morning.

The cardiac rhythm monitoring patch will be inspected. If the adhesive appears intact, the existing patch should be left in place. If the adhesive appears to be failing or the patch has become detached, a new patch will be applied at this time.

### Treatment Period 2 (5-8 Days):

From Visit 3 until Visit 4: Compound I BID will be given with food starting in the evening of the last day of Treatment Period 1 or the following morning depending on the results of SET on TTE performed at Visit 3.

Patient Contact 2: One to three days before end of Treatment Period 2 (V4), the subject should be contacted to ensure compliance with study treatment, to remind subject of scheduled time of next visit (Visit 4), and to take treatment (with food) in the morning of Visit 4 about 7 hours prior to the scheduled time of the visit.

Visit 4 (to be scheduled 5 to 9 days after V3, i.e. Day 9 (up to Day 15)): Subjects will return for a clinic visit in the afternoon for an assessment of safety, tolerability, PK and evaluation of PD response. The last dose of IMP for Treatment Period 2 will have been taken in the morning, approximately 7 hours before this clinic visit. Additional study assessments will be completed, including but not limited to laboratory and PK blood samples and 12-lead ECG (triplicate).

### Follow-Up

Patient Contact 3: The subject should be contacted 1 to 3 days following the last dose of IMP to assess safety.

Visit 5—A final study clinic visit to assess subject safety will be made 7 days (+1 day) following the last dose of IMP.

### Inclusion Criteria

This study is to be performed in patients who meet the following criteria:
1. Men or women 18 to 80 years of age at the Screening visit
2. Diagnosis of primary dilated cardiomyopathy (DCM), clinically stable and associated with MYH7 mutation as defined by all of the following:
   a. Primary DCM subjects with a diagnosis of heart failure with reduced ejection fraction that has no identified etiology other than MYH7 mutation (e.g., coronary artery disease or severe valvulopathy; presence of coronary artery disease, functional mitral regurgitation, or mild to moderate valvular disease may be allowed if not considered the primary cause of the heart failure);
   b. Pathogenic or likely pathogenic mutation in MYH7 gene;
   c. DCM is not secondary to long-standing MYH7-related hypertrophic cardiomyopathy (HCM) or LV noncompaction cardiomyopathy;
   d. Documented LVEF 15-40% (on two occasions, including at least once during Screening):
      If a subject's most recent prior TTE (within past 12 months) documents an LVEF≤40%, then only a single screening visit confirming LVEF≤40% is required;
      If no prior documented LVEF≤40% by TTE within past 12 months is available, then 2 screening TTEs are needed at least one week (7 days) apart;
      In addition, the absolute difference between the 2 LVEF values qualifying the subject should <12%;
   e. At least mild left ventricular enlargement by ASE criteria (LVEDD ≥3.1 cm/m² for males, ≥3.2 cm/m² for females);
   f. Subject receives chronic medication for the treatment of heart failure reflecting current guidelines, including at least one of the following, unless not tolerated or contraindicated: β-blocker, ACE inhibitor, ARB, or ARNI. Such treatments should have been given at stable doses for ≥2 weeks with no plan to modify during the study.
3. Sinus rhythm or stable atrial or ventricular pacing or persistent atrial fibrillation that is adequately rate-controlled to allow PD assessments by TTE.

### Exclusion Criteria

Patients who meet any of the following criteria will be excluded from the study:
1. Inadequate echocardiographic acoustic windows.
2. A patient has a QTcF interval >480 msec (Fridericia's correction, not attributable to ventricular pacing or prolonged QRS duration ≥120 msec, average of triplicate ECGs).
3. Subjects with known pathogenic mutation of another gene implicated in DCM in addition to an MYH7 mutation.
4. HFrEF that is considered to be caused primarily by ischemic heart disease, chronic valvulopathy, or another condition.
5. Recent (<90 days) acute coronary syndrome or angina pectoris.
6. Coronary revascularization (percutaneous coronary intervention [PCI] or coronary artery bypass graft [CABG]) within prior 90 days.
7. Recent (<90 days) hospitalization for heart failure, use of IV diuretic or chronic IV inotropic therapy or other cardiovascular event (e.g., cerebrovascular accident).
8. Known aortic stenosis of moderate or greater severity.
9. Presence of disqualifying cardiac rhythms that would preclude echocardiographic assessments, as determined by the Investigator, including: (a) rapid, inadequately rate-controlled atrial fibrillation or (b) frequent premature ventricular contractions that might interfere with reliable echocardiographic measurements of LV function.
10. Hypersensitivity to Compound I or any of the components of the Compound I formulation.
11. Active infection, indicated clinically.
12. History of malignancy of any type within 5 years prior to Screening, with the exception of the following surgically excised cancers occurring more than 2 years prior to Screening: in situ cervical cancer, nonmelanomatous skin cancers, ductal carcinoma in situ, and nonmetastatic prostate cancer.
13. Severe renal insufficiency (defined as current estimated glomerular filtration rate [eGFR]<30 mL/min/1.73 m² by simplified Modification of Diet in Renal Disease equation [sMDRD]).
14. Serum potassium <3.5 or >5.5 mEq/L.
15. Any persistent (2 or more) out-of-range safety laboratory parameters (chemistry, hematology), considered to be clinically significant.
16. History or evidence of any other clinically significant disorder, condition, or disease (including substance abuse) that would pose a risk to subject safety or interfere with the study evaluation, procedures, completion, or lead to premature withdrawal from the study.
17. A life expectancy of <6 months.
18. Participated in a clinical trial in which the subject received any investigational drug (or is currently using an investigational device) within 30 days prior to Screening, or at least 5 times the respective elimination half-life (whichever is longer).

### Study Treatment

Ambulatory stable MYH7-DCM subjects will participate in two sequential open-label treatment periods of 5 to 8 days each.

Compound I will be provided in 5 mg tablets (to support 10 mg and 25 mg dosings) and 25 mg tablets (to support the 50 mg dosing). The tablets will be blistered and then carded; each blister card will contain either only 5 mg or only 25 mg.

### Treatment Period 1

Subjects will receive 25 mg Compound I twice daily (every 12 hours). Doses may occur ±2 hours from scheduled dosing times as long as doses are separated by at least 10 hours and by no more than 14 hours for at least 5 and up to 8 days. The first dose will be ingested in the morning on Day 1 (morning) and last dose ingested in the morning, at the earliest on Day 5 and at the latest on Day 8 (corresponding to a total of 9 to 15 doses for Period 1). On the day of last dose of Treatment Period 1, an echocardiogram will be performed in the afternoon approximately 7 hours after the morning dose. The systolic ejection time (SET) change from baseline measured on that TTE by the sonographer at each local site will determine the dose to be administered in Treatment Period 2.

### Treatment Period 2

If at end of Period 1, SET change from baseline (D1, pre-dose) is >60 msec, subject will be instructed to skip 1 dose and be down-titrated to 10 mg BID.

If at end of Period 1, SET change from baseline (D1, pre-dose) is ≤60 msec, subject will be up-titrated to 50 mg BID.

First dose of Treatment Period 2 will start in the evening on the last day of Treatment Period 1 in the case of subjects being up-titrated and in the morning of the subsequent day in subjects being down-titrated. Dosing for Period 2 will last between 5 to 8 days and the last dose in Period 2 will be ingested in the morning, at the earliest on Day 9 and at the latest on Day 15 (corresponding to a total of 7 to 14 doses for Period 2).

For both treatment periods:
Subjects will be dosed twice daily (every 12 hours). Doses may occur ±2 hours from scheduled dosing times as long as doses are separated by at least 10 hours and not more than 14 hours.
Each dose will be ingested with a meal.
   The two treatment periods do not need to have the same duration.

### Management of an Exaggerated Pharmacological Effect

Based on the nonclinical pharmacological characteristics, exaggerated effects of Compound I may lead to myocardial ischemia. The duration of effect would follow the PK profile of Compound I with a Tmax of 4 to 6 hours and a half-life of approximately 15 hours in healthy volunteers, but a slightly longer half-life in subjects with HFrEF that received Compound I (20 to 25 hours). The clinical signs and symptoms, which may include chest pain, lightheadedness, diaphoresis, and ECG changes should start to abate over a short period of time. Any subject with signs and/or symptoms suggestive of cardiac ischemia should be immediately evaluated by the physician for the potential diagnosis of cardiac ischemia. The entire context including clinical symptoms, ECGs and serial cardiac biomarkers (e.g. troponin, CK-MB), and cardiac imaging (including coronary angiography, if applicable) should be considered in making that determination, since patients enrolled in the study are likely to have abnormal ECGs and possibly elevated or fluctuating troponin levels at baseline in relation to their heart failure condition. If evidence of cardiac ischemia is present, then the subject should receive standard therapy for ischemia as appropriate, including supplemental oxygen and nitrates. Caution in the administration of agents that increase HR is required, as Compound I may prolong the SET, which could result in decreasing the diastolic duration resulting in a decrease in diastolic ventricular filling. In addition, the exaggerated pharmacological effect may increase myocardial oxygen demand, so agents that may increase myocardial oxygen demand further should be administered with caution.

### Concomitant Therapy

During the study, subjects should continue to take their medications for the treatment of congestive heart failure and other medical conditions at the same doses and as close to the same times as usual, in order to maintain as best as possible similar preload and afterload conditions throughout the study and to minimize confounding factors for the assessment of the effects of Compound I.

All prescription and over-the-counter medications must be reviewed. Over-the-counter medications may be taken at stable doses throughout the study, and in amounts no greater than as directed per the label. Questions concerning enrollment or medications should be discussed with the medical monitor. Coadministration of Compound I with fluconazole (a strong CYP2C19 inhibitor and moderate inhibitor of CYP2C9 and CYP3A4) and rifampin (a strong inducer of CYP3A4, CYP2C19, and CYP2C9) should be avoided. Other investigational therapies must be discontinued at least 30 days prior to Screening or 5 half-lives (whichever is longer).

If the subject has an AE requiring treatment (including the ingestion of acetaminophen or ibuprofen), the medication should be recorded; including time of the administration (start/stop), date, dose, and indication.

### Study Assessments and Procedures

### I. Pharmacodynamic Assessments

The PD effect of Compound I will be evaluated throughout this study by serial TTE examination in accordance with a standardized imaging protocol and compared with baseline. Key TTE measurements will include but not be limited to:
Change in left ventricular systolic ejection time (SET)
Change in left ventricular systolic functional parameters
   Stroke volume (LVSV)
   Ejection fraction (LVEF)
   Global longitudinal strain (LVGLS) and circumferential strain (LVGCS)
LV end-systolic dimensions indexed for body surface area (LVEDVi, LVESVi)-Change in left ventricular diastolic parameters
   Tissue Doppler Imaging (TDI): mitral valve annular motion (e′)
   E/A ratio
   E/e′ ratio

Change in daily activity will be explored by tracking via a wearable device.

### II. Pharmacokinetic Assessments

Peak blood samples to measure Compound I (and potential metabolite) plasma concentration will be drawn.

### III. Genetic/Genotype/Pharmacogenetic/Biomarker Assessment

All subjects will be asked to provide consent for blood to be drawn for potential future analysis of genetic markers in relation to efficacy, safety, PD, or PK parameters as determined by future studies using clinically meaningful endpoints, through DNA genotyping, direct sequencing, or other genetic testing modalities unless there are local regulations prohibiting these analyses. If genetic or pharmacogenetic studies are conducted, genetic information will not be returned to subjects.

### IV. Pharmacodynamic Analyses

TTE data for all measured parameters will be analyzed using descriptive statistics. Change from baseline will be summarized at each time point. Observations by timepoint and change from Baseline (either absolute or percent relative change) at each timepoint will be summarized by treatment period). Change from Baseline will be analyzed with attention to the relationship to time postdose and dose level.

The relationship between the TTE endpoints and Compound I plasma concentration will be assessed using linear or nonlinear correlations.

### V. Pharmacokinetic Analyses

Plasma concentration data for Compound I at different doses will be summarized using descriptive statistics, including mean or geometric mean, as appropriate, standard deviation (SD), median, minimum and maximum values, and percent coefficient of variation (CV %).

### VI. Pharmacokinetic/Pharmacodynamic Analyses

Correlations of TTE parameters with Compound I plasma concentration will be assessed. It is anticipated that each subject will provide PK and PD data at two levels of drug exposure from the last dosing days of both Treatment Period 1 and 2.

### VII. Troponin Analyses

The number of subjects with abnormal and/or rising troponin levels (taking into account potential troponin elevation at baseline) will be determined. Abnormal and/or rising troponin values (taking into account potential baseline troponin elevation frequently observed in heart failure) should lead to the subject being clinically evaluated for possible myocardial ischemia. Also, if the subject has any signs or symptoms suggestive of possible cardiac ischemia, additional serial troponin (and other safety labs, including CK-MB samples) should be obtained and subsequent dosing should be withheld until there is full understanding of the possible ischemic event. The entire clinical context (e.g., signs, symptoms, new ECG changes, new troponin, and CK-MB abnormalities) should be evaluated and correlated with any other relevant clinical findings, subject's medical history, and laboratory data to determine the clinical significance of the findings.

### VIII. Safety Analyses

AEs, ECGs, vital signs, and laboratory values will be analyzed using descriptive statistics.

### IX. Exploratory Analysis

Change in daily activity level will be measured by wearable device and could be summarized using descriptive statistics.

### X. Subject Restrictions During the Study

Starting at Screening and throughout the study, subjects should be instructed to maintain a stable lifestyle. This includes but is not limited to:
Concomitant medications: every effort should be taken to maintain stable doses of concomitant medications, and to take such medications at consistent times during the day; for cardiovascular drugs, this will allow to minimize variability in cardiac loading conditions.
Activity levels; from 72 hours prior to the first dose through the final Follow-up visit, subjects should not engage in unaccustomed intensive exercise.
Meals: to be taken as best as possible at consistent times during the day (with Compound I taken with a meal twice a day).
Abstain from grapefruit or grapefruit juice, Seville oranges, and quinine (e.g., tonic water).
Fluid intake: avoid excessive fluid intake or excessive alcohol consumption.

In addition, starting at Screening, subjects will be required to abstain from blood or plasma donation until 3 months after the final study visit.

### Study Endpoints

Primary endpoints are clinical safety and tolerability as assessed with the following:
Treatment-emergent AEs and SAEs, and
Clinically significant abnormalities from vital signs, physical examination, ECG recordings, and safety labs.

Secondary endpoints include the following PD parameters as assessed by TTE:
Systolic ejection time,
Parameters of left ventricular systolic function including but not limited to LVSV, LVEF, LVESV, and LV strain will be evaluated, and
Parameters of left ventricular diastolic function including but not limited to TDI (e′), E/A, and E/e′ will be evaluated.

Exploratory endpoints are:
Daily activity level measured by accelerometer, and
Additional exploratory endpoints including PK may be included.

1-53. (canceled)

54. A method of treating dilated cardiomyopathy (DCM) in a subject in need thereof, comprising:
administering to the subject a first dose of danicamtiv or a pharmaceutically acceptable salt thereof for a first treatment period;
evaluating, or having evaluated, a systolic ejection time (SET) prolongation of the subject following the first treatment period;
selecting, or having selected, a second dose of danicamtiv or a pharmaceutically acceptable salt thereof, wherein the second dose is different than the first dose, based on the evaluation of the SET prolongation;
administering the second dose of danicamtiv or a pharmaceutically acceptable salt thereof to the subject for a second treatment period.

55. The method of claim 54, wherein the evaluating the SET prolongation of the subject comprises comparing the SET prolongation of the subject to a threshold and determining that the SET prolongation of the subject is above or below a threshold.

56. The method of claim 55, wherein the SET prolongation of the subject following the first treatment period is above the threshold and wherein the second dose is lower than the first dose.

57. The method of claim 56, wherein the first dose is 25 mg twice daily and the second dose is 10 mg twice daily.

58. The method of claim 55, wherein the SET prolongation of the subject following the first treatment period is below the threshold and wherein the second dose is higher than the first dose.

59. The method of claim 58, wherein the first dose is 25 mg twice daily and the second dose is 50 mg twice daily.

60. The method of claim 55, wherein the threshold is 60 milliseconds.

61. The method of claim 55, wherein the threshold is 75 milliseconds.

62. The method of claim 55, wherein the threshold is 50 milliseconds.

63. The method of claim 55, wherein the SET prolongation of the subject comprises an SET prolongation measurement on a single echocardiogram or two SET prolongation measurements on two consecutive echocardiogram.

64. The method of claim 54, wherein the first dose is administered twice daily.

65. The method of claim 64, wherein the second dose is administered twice daily.

66. The method of claim 54, wherein the first dose is from about 10 mg to about 75 mg twice daily.

67. The method of claim 66, wherein the second dose is from about 10 mg to about 75 mg twice daily, and is different than the first dose.

68. The method of claim 66, wherein the first dose is 25 mg twice daily.

69. The method of claim 68, wherein the second dose is 10 or 50 mg twice daily.

70. The method of claim 54, wherein the first treatment period is from about 5 to about 8 days.

71. The method of claim 70, wherein the second treatment period is from about 5 to about 8 days.

72. The method of claim 54, wherein evaluating SET prolongation is determined by transthoracic echocardiogram (TTE).

73. The method of claim 55, further comprising skipping administration of a dose of danicamtiv or a pharmaceutically acceptable salt thereof after the first treatment period if the SET prolongation is above a threshold.

74. The method of claim 54, wherein the DCM is genetic DCM.

75. The method of claim 74, wherein the genetic DCM is associated with a MYH7 mutation.

76. The method of claim 74, wherein the genetic DCM is associated with a genetic mutation in a gene selected from ABCC9, ACTC1, ACTN2, ANKRD1, BAG3, CRYAB, CSRP3, DES, DMD, DSG2, EYA4, GATAD1, LAMA4, LDB3, LMNA, MYBPC3, MYH6, MYH7, MYPN, PLN, PSEN1, PSEN2, RBM20, SCN5A, SGCD, TAZ, TCAP, TMPO, TNNC1, TNNI3, TNNT2, TPM1, TTN, VCL, or any combination thereof.

77. The method of claim 74, wherein the genetic DCM is associated with a genetic mutation in a gene selected from ACTC1, DES, MYH6, MYH7, TNNC1, TNNI3, TNNT2, TTN, or any combination thereof.

78. The method of claim 74, wherein the genetic DCM is associated with a TTN mutation.

79. A method of treating a disease or disorder selected from systolic dysfunction, heart failure, or cardiomyopathy, in a subject in need thereof, the method comprising:
administering to the subject a first dose of danicamtiv or a pharmaceutically acceptable salt thereof for a first treatment period;
evaluating, or having evaluated, a systolic ejection time (SET) prolongation of the subject following the first treatment period;
selecting, or having selected, a second dose of danicamtiv or a pharmaceutically acceptable salt thereof, wherein the second dose is different than the first dose, based on the evaluation of the SET prolongation;
administering the second dose of danicamtiv or a pharmaceutically acceptable salt thereof to the subject for a second treatment period.

80. The method of claim 79, wherein the disease or disorder is systolic dysfunction.

81. The method of claim 79, wherein the disease or disorder is heart failure.

82. The method of claim 81, wherein the heart failure is heart failure with reduced ejection fraction (HFrEF).
